SEQUENCE LISTING (1 sequence)

SEQ ID NO: 1 - protein, 20 aa, Artificial Sequence
  Synthetic Peptide
  MOD_RES (20)..(20): AMIDATION
KKKKKEQEDEPEGDYFEWLE

(19) United States
(12) Patent Application Publication
(10) Pub. No.: US 2024/0285621 A1
(43) Pub. Date: Aug. 29, 2024

(54) PYRAZOLYL-PYRIMIDINE DERIVATIVES AS KINASE INHIBITORS

(71) Applicant: NERVIANO MEDICAL SCIENCES S.R.L., Nerviano (MI) (IT)

(72) Inventors: Simona BINDI, Milan (IT); Laura BUFFA, Rossiglione (GE) (IT); Giovanni CERVI, Como (IT); Roberto D'ALESSIO, Cinisello Balsamo (MI) (IT); Maria MENICHINCHERI, Milan (IT); Michele MODUGNO, Parabiago (MI) (IT)

(73) Assignee: NERVIANO MEDICAL SCIENCES S.R.L., Nerviano (MI) (IT)

(21) Appl. No.: 18/027,489

(22) PCT Filed: Sep. 15, 2021
(86) PCT No.: PCT/EP2021/075297

§ 371 Date: Mar. 21, 2023

(30) Foreign Application Priority Data
Sep. 23, 2020 (EP) 20197710.5

(51) Int. Cl. A61K 31/506; A61K 31/5377; A61K 45/06; C07D 401/14; C07D 403/04; C07D 403/14; C07D 405/14; C07D 453/02

(52) U.S. Cl. CPC A61K 31/506; A61K 31/5377; A61K 45/06; C07D 401/14; C07D 403/04; C07D 403/14; C07D 405/14; C07D 453/02

(57) ABSTRACT

The present invention relates to pyrazolyl-pyrimidine derivatives, to a process for their preparation, to pharmaceutical compositions comprising them, and to their use as therapeutic agents. The compounds are kinase inhibitors, in particular are inhibitors of Spleen Tyrosine Kinase (SYK) and can be used in treatment of cancer, cell proliferative disorders, viral infections, immune disorders, neurodegenerative disorders and cardiovascular diseases.

The present invention relates to pyrazolyl-pyrimidine derivatives, to a process for their preparation, to pharmaceutical compositions comprising them, and to their use as therapeutic agents, particularly in the treatment of diseases caused by dysregulated protein kinase activity, such as cancer, cell proliferative disorders, viral infections, immune disorders, neurodegenerative disorders and cardiovascular diseases.

### BACKGROUND OF THE INVENTION

The malfunctioning of protein kinases (PKs) is the hallmark of numerous diseases. A large share of the oncogenes and proto-oncogenes involved in human cancers encode for PKs. The enhanced activities of PKs are also implicated in many non-malignant diseases, such as benign prostate hyperplasia, familial adenomatosis, polyposis, neurofibromatosis, psoriasis, vascular smooth cell proliferation associated with atherosclerosis, pulmonary fibrosis, arthritis glomerulonephritis and post-surgical stenosis and restenosis.

PKs are also implicated in inflammatory conditions and in the multiplication of viruses and parasites. PKs may also play a major role in the pathogenesis and development of neurodegenerative disorders.

For a general reference to PKs malfunctioning or deregulation see, for instance, Current Opinion in Chemical Biology, 1999, 3, 459-465; Nature Rev. Drug Discov. 2002; and Carcinogenesis, 2008, 29, 1087-1091.

Spleen tyrosine kinase (Syk) is a 72 kDa non-receptor cytoplasmic tyrosine kinase. Syk has a primary amino acid sequence similar to that of zeta-associated protein-70 (ZAP-70) and is involved in receptor-mediated signal transduction. The N-terminal domain of Syk contains two Src-homology 2 (SH2) domains, which bind to diphosphorylated immunoreceptor tyrosine-based activation motifs (ITAMs) found in the cytoplasmic signaling domains of many immunoreceptor complexes. The C-terminus contains the catalytic domain. Syk is expressed in many cell types involved in adaptive and innate immunity, including lymphocytes (B cells, T cells, and NK cells), granulocytes (basophils, neutrophils, and eosinophils), monocytes, macrophages, dendritic cells, and mast cells. Syk plays critical roles in immunoreceptor-mediated signaling in a variety of cell types, including B-cells, macrophages, monocytes, mast cells, eosinophils, basophils, neutrophils, dendritic cells, platelets, and osteoclasts. Classical immunoreceptors include B-cell and T-cell antigen receptors as well as various immunoglobulin receptors (Fc receptors). Ligand binding leads to activation of immunoreceptors, which results in Src family kinases being activated, and phosphorylation of immunoreceptor tyrosine-based activation motifs (ITAMs) in the cytoplasmic face of receptor-associated transmembrane adaptors. Syk binds to the phosphorylated ITAM motifs of the adaptors, leading to activation of Syk and subsequent phosphorylation and activation of downstream signaling pathways (Mocsai et al., 2010: 10(6):387).

Syk is essential for B-cell activation through B-cell receptor (BCR) signaling. Syk becomes activated upon binding to phosphoryated BCR and thus initiates the early signaling events following BCR activation. A large body of evidence shows that BCR signalling also supports the survival and growth of malignant B cells in patients with B cell leukaemias or lymphomas. The mechanism of BCR pathway activation in these diseases includes continuous BCR stimulation by microbial antigens or autoantigens present in the tissue microenvironment, activating mutations within the BCR complex or downstream signaling components and ligand-independent tonic BCR signaling. Targeting key BCR-signalling mediators (such as Btk or the delta isoform of PI3K) with small molecules has demonstrated to be a valuable approach to inhibit the BCR signaling resulting in a therapeutic benefit in these diseases (Burger and Wiestner, Nat. Rev. Cancer 2018: 18(3):148).

Thus, the inhibition of Syk activity can be useful for the treatment of certain types of cancers in which BCR-signalling plays a role for their survival and proliferation, such as Non-Hodgkin's lymphomas, chronic lymphocytic leukemia, acute myeloid leukemia, acute lymphocytic leukemia, T-cell lymphomas. In addition, Syk's role in ITAM-dependent signaling and its expression in many cell types suggest that compounds which inhibit Syk activity may be useful for treating disorders involving the immune system and inflammation. Such disorders include Type I hypersensitivity reactions (allergic rhinitis, allergic asthma, and atopic dermatitis); autoimmune diseases (rheumatoid arthritis, multiple sclerosis, systemic lupus erythematosus, psoriasis, and immune thrombocytopenic purpura); (Pamuk and Tsokos, Arthritis Res Ther. 2010; 12(6):222.

Pyrazolylpyrimidine derivatives were previously described as modulator of protein kinases activity and are therefor useful in treating diseases caused by dysregulated protein kinase activity (WO 2012/139930).

Pyrimidine-5-carboxamido compounds acting as inhibitors of Syk and/or JAK kinase are described in the patent application US20200239458 while solid forms of condensed pyrazines as Syk inhibitors are described in WO2020172431.

### DETAILED DESCRIPTION OF THE INVENTION

The present inventors have discovered that compounds of general formula (I), as defined below, are kinase inhibitors and in particular are inhibitors of Spleen Tyrosine Kinase (SYK). Such compounds are thus useful to treat diseases caused by altered SYK activity.

Accordingly, a first object of the present invention is to provide a substituted pyrazolo-pyrimidine derivative of formula (I):

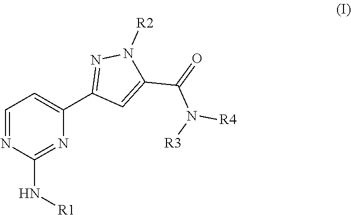

wherein:
R1 is hydrogen or an optionally substituted group selected from straight or branched (C1-C6) alkyl, (C3-C7) cycloalkyl, aryl, heterocyclyl and heteroaryl;
R2 is hydrogen or an optionally substituted group selected from straight or branched (C1-C6) alkyl, (C2-C6) alkenyl, (C2-C6) alkynyl, and (C3-C7) cycloalkyl;
R3 and R4 are independently hydrogen, straight or branched (C1-C6) alkyl optionally substituted with halogens, heteroaryl or heteroaryl (C1-C6) alkyl or a group of formula (II):

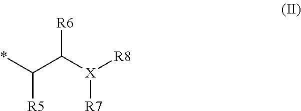

wherein:
R5 is hydrogen, an optionally substituted straight or branched (C1-C6) alkyl, (C3-C7) cycloalkyl, aryl or taken together with R6 may form an optionally substituted 4- to 7 membered cycloalkyl or, taken together with R7 or R8, may form an optionally substituted heterocyclyl group;
R6 is hydrogen or methyl or taken together with R3 or R4 may form an optionally substituted 4- to 7 membered heterocyclic group;
R7 and R8 are independently hydrogen, an optionally substituted straight or branched (C1-C6) alkyl or may form together with X an optionally substituted 4- to 7 membered heterocyclyl group optionally containing one additional heteroatom selected from N, O and S or may form together with R3 or R4 an optionally substituted 5- to 7 membered heterocyclyl group;
X is H, N or O
provided that, when X is O, R5 is not phenyl;
or a pharmaceutically acceptable salt thereof.

Preferred compounds of formula (I) are the compounds wherein:
R2 is an optionally substituted straight or branched (C1-C6) alkyl;
R3 and R4 are independently hydrogen or a group of general formula (II)

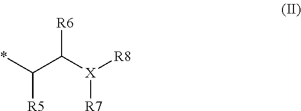

wherein:
R5 is hydrogen, an optionally substituted straight or branched (C1-C6)alkyl, (C3-C7)cycloalkyl or phenyl or, taken together with R6 may form an optionally substituted 4- to 7 membered cycloalkyl;
R6 is hydrogen;
R7 and R8 are independently hydrogen, an optionally substituted straight or branched (C1-C6)alkyl or may form together with X an optionally substituted 4- to 7 membered heterocyclyl group optionally containing one additional heteroatom selected from N, O and S;
X is N or O;
R1 is as defined above;
provided that, when X is O, R5 is not phenyl;
or a pharmaceutically acceptable salt thereof.

Further preferred compounds of formula (I) are the compounds wherein:
R3 is hydrogen or straight or branched (C1-C6) alkyl optionally substituted with halogens, heteroaryl or heteroaryl (C1-C6) alkyl;
R4 is hydrogen or a group of general formula (II)

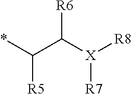

wherein:
R5 is hydrogen, an optionally substituted straight or branched (C1-C6)alkyl, (C3-C7)cycloalkyl or phenyl or, taken together with R6 may form an optionally substituted 4- to 7 membered cycloalkyl;
R6 is hydrogen;
R7 and R8 are independently hydrogen, an optionally substituted straight or branched (C1-C6)alkyl or may form together with X an optionally substituted 4- to 7 membered heterocyclyl group optionally containing one additional heteroatom selected from N, O and S;
X is N or O;
R1 and R2 are as defined above;
provided that, when X is O, R5 is not phenyl;
or a pharmaceutically acceptable salt thereof.

Even more preferred are compounds of formula (I) wherein:
R1 is an optionally substituted aryl, heterocyclyl or heteroaryl group;
R3 is hydrogen;
R4 is a group of general formula (II):

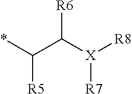

wherein:
R5 is hydrogen, an optionally substituted straight or branched (C1-C6)alkyl, (C3-C7)cycloalkyl or phenyl or taken together with R6 may form an optionally substituted 4- to 7 membered cycloalkyl;
R6 is hydrogen;
R7 and R8 are independently hydrogen, an optionally substituted straight or branched (C1-C6)alkyl or may form together with X an optionally substituted 4- to 7 membered heterocyclyl;
X is N;
R2 is as defined above;
or a pharmaceutically acceptable salt thereof.

Further even more preferred are compounds of formula (I) wherein:
R1 is an optionally substituted group selected from phenyl or indolyl;
R2, R3 and R4 are as defined above;
or a pharmaceutically acceptable salt thereof.

Preferred specific compounds (cpd) of formula (I) or a salt thereof are the compounds listed below:
N-[2-(dimethylamino)ethyl]-3-{2-[(3,5-dimethylphenyl)amino]pyrimidin-4-yl}-1-methyl-1H-pyrazole-5-carboxamide (cpd 1);
3-{2-[(3,5-dimethylphenyl)amino]pyrimidin-4-yl}-N-[(2R)-1-hydroxypropan-2-yl]-1-methyl-1H-pyrazole-5-carboxamide (cpd 2);
3-{2-[(3,5-dimethylphenyl)amino]pyrimidin-4-yl}-N-[(2S)-1-hydroxypropan-2-yl]-1-methyl-1H-pyrazole-5-carboxamide (cpd 3);
3-{2-[(3,5-dimethylphenyl)amino]pyrimidin-4-yl}-1-methyl-N-(propan-2-yl)-1H-pyrazole-5-carboxamide (cpd 4);
N-[2-(dimethylamino)ethyl]-3-{2-[(3,5-dimethylphenyl)amino]pyrimidin-4-yl}-N,1-dimethyl-1H-pyrazole-5-carboxamide (cpd 5);
3-{2-[(3,5-dimethylphenyl)amino]pyrimidin-4-yl}-1-methyl-N-[2-(methylamino)ethyl]-1H-pyrazole-5-carboxamide hydrochloride (cpd 6);
N-(2-aminoethyl)-3-{2-[(3,5-dimethylphenyl)amino]pyrimidin-4-yl}-1-methyl-1H-pyrazole-5-carboxamide hydrochloride (cpd 7);
N-(azetidin-3-yl)-3-{2-[(3,5-dimethylphenyl)amino]pyrimidin-4-yl}-1-methyl-1H-pyrazole-5-carboxamide trifluoroacetate (cpd 8);
3-{2-[(3,5-dimethylphenyl)amino]pyrimidin-4-yl}-1-methyl-N-[2-(morpholin-4-yl)ethyl]-1H-pyrazole-5-carboxamide (cpd 9);
N-[2-(diethylamino)ethyl]-3-{2-[(3,5-dimethylphenyl)amino]pyrimidin-4-yl}-1-methyl-1H-pyrazole-5-carboxamide (cpd 10);
N-[(1R,2R)-2-aminocyclohexyl]-3-{2-[(3,5-dimethylphenyl)amino]pyrimidin-4-yl}-1-methyl-1H-pyrazole-5-carboxamide hydrochloride (cpd 11);
3-{2-[(3,5-dimethylphenyl)amino]pyrimidin-4-yl}-1-methyl-N-[2-(propan-2-ylamino)ethyl]-1H-pyrazole-5-carboxamide hydrochloride (cpd 12);
3-{2-[(3,5-dimethylphenyl)amino]pyrimidin-4-yl}-1-methyl-1H-pyrazole-5-carboxamide (cpd 13);
3-{2-[(3,5-dimethylphenyl)amino]pyrimidin-4-yl}-N,1-dimethyl-1H-pyrazole-5-carboxamide (cpd 14);
3-{2-[(3,5-dimethylphenyl)amino]pyrimidin-4-yl}-N,N,1-trimethyl-1H-pyrazole-5-carboxamide (cpd 15);
3-{2-[(3,5-dimethylphenyl)amino]pyrimidin-4-yl}-N-(2-methoxyethyl)-1-methyl-1H-pyrazole-5-carboxamide (cpd 16);
3-{2-[(3,5-dimethylphenyl)amino]pyrimidin-4-yl}-N-(2-fluoroethyl)-1-methyl-1H-pyrazole-5-carboxamide (cpd 17);
3-(2-{[3,5-bis(trifluoromethyl)phenyl]amino}pyrimidin-4-yl)-N-[2-(dimethylamino)ethyl]-1-methyl-1H-pyrazole-5-carboxamide hydrochloride (cpd 18);
3-(2-{[3,5-bis(trifluoromethyl)phenyl]amino}pyrimidin-4-yl)-N-[(2S)-1-hydroxypropan-2-yl]-1-methyl-1H-pyrazole-5-carboxamide (cpd 19);
3-{2-[(3,5-dimethylphenyl)amino]pyrimidin-4-yl}-1-methyl-N-(1H-pyrazol-3-yl)-1H-pyrazole-5-carboxamide (cpd 20);
(3-{2-[(3,5-dimethylphenyl)amino]pyrimidin-4-yl}-1-methyl-1H-pyrazol-5-yl)(4-methylpiperazin-1-yl)methanone (cpd 21);
N-[2-(acetylamino)ethyl]-3-{2-[(3,5-dimethylphenyl)amino]pyrimidin-4-yl}-1-methyl-1H-pyrazole-5-carboxamide (cpd 22);
N-(2-amino-2-oxoethyl)-3-{2-[(3,5-dimethylphenyl)amino]pyrimidin-4-yl}-1-methyl-1H-pyrazole-5-carboxamide (cpd 23);
3-{2-[(3,5-dimethylphenyl)amino]pyrimidin-4-yl}-1-methyl-N-(2,2,2-trifluoroethyl)-1H-pyrazole-5-carboxamide (cpd 24);
3-{2-[(3,5-dimethylphenyl)amino]pyrimidin-4-yl}-1-methyl-N-[(1-methyl-1H-imidazol-5-yl)methyl]-1H-pyrazole-5-carboxamide (cpd 25);
3-{2-[(3,5-dimethylphenyl)amino]pyrimidin-4-yl}-N-[(2S)-1-hydroxy-3-methylbutan-2-yl]-1-methyl-1H-pyrazole-5-carboxamide (cpd 26);
3-{2-[(3,5-dimethylphenyl)amino]pyrimidin-4-yl}-1-methyl-N-(pyridin-2-ylmethyl)-1H-pyrazole-5-carboxamide (cpd 27);
3-{2-[(3,5-dimethylphenyl)amino]pyrimidin-4-yl}-N-(1H-imidazol-2-ylmethyl)-1-methyl-1H-pyrazole-5-carboxamide (cpd 28);
N-[(2R)-1-(azetidin-1-yl)propan-2-yl]-3-{2-[(3,5-dimethoxyphenyl)amino]pyrimidin-4-yl}-1-methyl-1H-pyrazole-5-carboxamide (cpd 29);
N-[(2S)-1-(dimethylamino)propan-2-yl]-1-methyl-3-[2-(tetrahydro-2H-pyran-4-ylamino)pyrimidin-4-yl]-1H-pyrazole-5-carboxamide (cpd 30);
N-(1-azabicyclo[2.2.2]oct-3-yl)-3-{2-[(3,5-dimethylphenyl)amino]pyrimidin-4-yl}-1-methyl-1H-pyrazole-5-carboxamide (cpd 31);
5-[2-(3,5-Dimethyl-phenylamino)-pyrimidin-4-yl]-2-methyl-2H-pyrazole-3-carboxylic acid ((1R,2R)-2-hydroxy-cyclohexyl)-amide (cpd 32);
3-{2-[(3,5-dimethylphenyl)amino]pyrimidin-4-yl}-N-[(2S)-1-hydroxybutan-2-yl]-1-methyl-1H-pyrazole-5-carboxamide (cpd 33);
3-{2-[(3,5-dimethylphenyl)amino]pyrimidin-4-yl}-N-(2-hydroxyethyl)-1-methyl-1H-pyrazole-5-carboxamide (cpd 34);
N-[(2S)-1-(azetidin-1-yl)propan-2-yl]-3-{2-[(3,4-dimethoxyphenyl)amino]pyrimidin-4-yl}-1-methyl-1H-pyrazole-5-carboxamide (cpd 35);
(3-{2-[(3,5-dimethylphenyl)amino]pyrimidin-4-yl}-1-methyl-1H-pyrazol-5-yl)(piperazin-1-yl)methanone hydrochloride (cpd 36);
N-[(1S,2R)-2-aminocyclohexyl]-3-(2-{[3-methoxy-5-(trifluoromethyl)phenyl]amino}pyrimidin-4-yl)-1-methyl-1H-pyrazole-5-carboxamide hydrochloride (cpd 37);
N-[(1S,2R)-2-aminocyclohexyl]-3-{2-[(3-chloro-1-methyl-1H-indol-5-yl)amino]pyrimidin-4-yl}-1-methyl-1H-pyrazole-5-carboxamide hydrochloride (cpd 38);
N-[(2S)-1-(azetidin-1-yl)propan-2-yl]-3-{2-[(3-methoxyphenyl)amino]pyrimidin-4-yl}-1-methyl-1H-pyrazole-5-carboxamide (cpd 39);
N-[(2S)-1-(azetidin-1-yl)propan-2-yl]-3-{2-[(3,5-difluorophenyl)amino]pyrimidin-4-yl}-1-methyl-1H-pyrazole-5-carboxamide (cpd 40);
N-[(2S)-1-hydroxy-3-methylbutan-2-yl]-1-methyl-3-{2-[(3-methylphenyl)amino]pyrimidin-4-yl}-1H-pyrazole-5-carboxamide (cpd 41);
3-{2-[(3,5-dimethylphenyl)amino]pyrimidin-4-yl}-1-methyl-N-[(2S)-1,1,1-trifluoropropan-2-yl]-1H-pyrazole-5-carboxamide (cpd 42);
N-[3-(dimethylamino)propyl]-3-{2-[(3,5-dimethylphenyl)amino]pyrimidin-4-yl}-1-methyl-1H-pyrazole-5-carboxamide (cpd 43);
(3-{2-[(3,5-dimethylphenyl)amino]pyrimidin-4-yl}-1-methyl-1H-pyrazol-5-yl)[(2S)-2-(propan-2-yl)aziridin-1-yl]methanone (cpd 44);
3-{2-[(3,5-dimethylphenyl)amino]pyrimidin-4-yl}-N-(2,2-dimethylpropyl)-1-methyl-1H-pyrazole-5-carboxamide (cpd 45);
3-{2-[(3,5-dimethylphenyl)amino]pyrimidin-4-yl}-1-methyl-N-(2-methylpropyl)-1H-pyrazole-5-carboxamide (cpd 46);
N-(cyclopropylmethyl)-3-{2-[(3,5-dimethylphenyl)amino]pyrimidin-4-yl}-1-methyl-1H-pyrazole-5-carboxamide (cpd 47);
N-[(2S)-1-(azetidin-1-yl)propan-2-yl]-3-{2-[(3-fluoro-5-methoxyphenyl)amino]pyrimidin-4-yl}-1-methyl-1H-pyrazole-5-carboxamide (cpd 48);
3-{2-[(4,6-dimethylpyridin-2-yl)amino]pyrimidin-4-yl}-N-[(2S)-1-hydroxy-3-methylbutan-2-yl]-1-methyl-1H-pyrazole-5-carboxamide (cpd 49);
3-{2-[(3,5-difluorophenyl)amino]pyrimidin-4-yl}-1-methyl-N-[(2S)-1,1,1-trifluoropropan-2-yl]-1H-pyrazole-5-carboxamide (cpd 50);
1-methyl-3-{2-[(3-methylphenyl)amino]pyrimidin-4-yl}-N-[(2S)-1,1,1-trifluoropropan-2-yl]-1H-pyrazole-5-carboxamide (cpd 51);
3-(2-{[3-chloro-4-(4-methylpiperazin-1-yl)phenyl]amino}pyrimidin-4-yl)-N-[(2S)-1-hydroxy-3-phenylpropan-2-yl]-1-methyl-1H-pyrazole-5-carboxamide (cpd 52);
N-[(1S,2S)-2-aminocyclohexyl]-1-methyl-3-{2-[(3-methylphenyl)amino]pyrimidin-4-yl}-1H-pyrazole-5-carboxamide (cpd 53);
3-(2-{[3-chloro-4-(4-methylpiperazin-1-yl)phenyl]amino}pyrimidin-4-yl)-1-methyl-N-[(2S)-1,1,1-trifluoropropan-2-yl]-1H-pyrazole-5-carboxamide (cpd 54);
3-{2-[(3,5-dimethylphenyl)amino]pyrimidin-4-yl}-1-methyl-N-[(2S)-1,1,1-trifluorobutan-2-yl]-1H-pyrazole-5-carboxamide (cpd 55);
3-{2-[(3,5-dimethylphenyl)amino]pyrimidin-4-yl}-1-methyl-N-[(2R)-1,1,1-trifluorobutan-2-yl]-1H-pyrazole-5-carboxamide (cpd 56);
N-[(2S)-1-(3,3-difluoroazetidin-1-yl)propan-2-yl]-3-{2-[(3,5-dimethylphenyl)amino]pyrimidin-4-yl}-1-methyl-1H-pyrazole-5-carboxamide (cpd 57);
1-methyl-N-[(2S)-1-(pyrrolidin-1-yl)propan-2-yl]-3-{2-[(3,4,5-trimethoxyphenyl)amino]pyrimidin-4-yl}-1H-pyrazole-5-carboxamide (cpd 58);
N-[(2S)-1-(azetidin-1-yl)propan-2-yl]-3-{2-[(3-chloro-1-methyl-1H-indol-5-yl)amino]pyrimidin-4-yl}-1-methyl-1H-pyrazole-5-carboxamide (cpd 59);
N-[(2S)-1-(azetidin-1-yl)propan-2-yl]-3-{2-[(3,5-dichlorophenyl)amino]pyrimidin-4-yl}-1-methyl-1H-pyrazole-5-carboxamide (cpd 60);
N-[(2S)-1-(azetidin-1-yl)-3-methylbutan-2-yl]-3-{2-[(3,5-dichlorophenyl)amino]pyrimidin-4-yl}-1-methyl-1H-pyrazole-5-carboxamide (cpd 61);
N-[(2S)-1-(azetidin-1-yl)propan-2-yl]-1-methyl-3-(2-{[3-(trifluoromethyl)phenyl]amino}pyrimidin-4-yl)-1H-pyrazole-5-carboxamide (cpd 62);
N-[(2S)-1-(azetidin-1-yl)propan-2-yl]-3-(2-{[3,5-bis(trifluoromethyl)phenyl]amino}pyrimidin-4-yl)-1-methyl-1H-pyrazole-5-carboxamide (cpd 63);
N-[(2S)-1-(azetidin-1-yl)propan-2-yl]-3-{2-[(3,5-dimethoxyphenyl)amino]pyrimidin-4-yl}-1-methyl-1H-pyrazole-5-carboxamide (cpd 64);
N-[(2S)-1-(dimethylamino)propan-2-yl]-3-{2-[(3-fluoro-5-methoxyphenyl)amino]pyrimidin-4-yl}-1-methyl-1H-pyrazole-5-carboxamide (cpd 65);
N-[(2S)-1-(azetidin-1-yl)propan-2-yl]-3-[2-(1,3-benzodioxol-5-ylamino)pyrimidin-4-yl]-1-methyl-1H-pyrazole-5-carboxamide (cpd 66);
3-{2-[(3,5-dimethoxyphenyl)amino]pyrimidin-4-yl}-N-[(2S)-1-(dimethylamino)propan-2-yl]-1-methyl-1H-pyrazole-5-carboxamide (cpd 67);
N-[(2S)-1-(azetidin-1-yl)propan-2-yl]-1-methyl-3-{2-[(3,4,5-trimethoxyphenyl)amino]pyrimidin-4-yl}-1H-pyrazole-5-carboxamide (cpd 68);
3-{2-[(3,5-dimethoxyphenyl)amino]pyrimidin-4-yl}-1-methyl-N-[(2S)-1-(pyrrolidin-1-yl)propan-2-yl]-1H-pyrazole-5-carboxamide (cpd 69);
3-{2-[(3-methoxyphenyl)amino]pyrimidin-4-yl}-1-methyl-N-[(2S)-1-(pyrrolidin-1-yl)propan-2-yl]-1H-pyrazole-5-carboxamide (cpd 70);
3-{2-[(3,5-dimethoxyphenyl)amino]pyrimidin-4-yl}-N-[(2S)-1-(dimethylamino)-1-oxopropan-2-yl]-1-methyl-1H-pyrazole-5-carboxamide (cpd 71);
3-{2-[(3,5-dimethoxyphenyl)amino]pyrimidin-4-yl}-N-[2-(dimethylamino)ethyl]-1-methyl-1H-pyrazole-5-carboxamide (cpd 72);
N-[(2S)-1-(dimethylamino)propan-2-yl]-3-{2-[(1,5-dimethyl-1H-pyrazol-3-yl)amino]pyrimidin-4-yl}-1-methyl-1H-pyrazole-5-carboxamide (cpd 73);
N-[(2S)-1-(azetidin-1-yl)propan-2-yl]-3-{2-[(1,5-dimethyl-1H-pyrazol-3-yl)amino]pyrimidin-4-yl}-1-methyl-1H-pyrazole-5-carboxamide (cpd 74);
N-[(2S)-1-(dimethylamino)propan-2-yl]-1-methyl-3-{2-[(1-methyl-1H-pyrazol-4-yl)amino]pyrimidin-4-yl}-1H-pyrazole-5-carboxamide (cpd 75);
N-[(2S)-1-(azetidin-1-yl)propan-2-yl]-1-methyl-3-[2-(tetrahydro-2H-pyran-4-ylamino)pyrimidin-4-yl]-1H-pyrazole-5-carboxamide (cpd 76);
N,N,1-trimethyl-3-[2-(tetrahydro-2H-pyran-4-ylamino)pyrimidin-4-yl]-1H-pyrazole-5-carboxamide (cpd 77);
3-{2-[(3-cyano-5-methoxyphenyl)amino]pyrimidin-4-yl}-N-[(2S)-1-(dimethylamino)propan-2-yl]-1-methyl-1H-pyrazole-5-carboxamide (cpd 78);
N-[(2S)-1-(azetidin-1-yl)propan-2-yl]-3-{2-[(3-cyano-5-methoxyphenyl)amino]pyrimidin-4-yl}-1-methyl-1H-pyrazole-5-carboxamide (cpd 79);
N-[(2S)-1-(azetidin-1-yl)propan-2-yl]-3-{2-[(5-methoxypyridin-3-yl)amino]pyrimidin-4-yl}-1-methyl-1H-pyrazole-5-carboxamide (cpd 80);
3-{2-[(3,5-dimethylphenyl)amino]pyrimidin-4-yl}-1-methyl-N-[(3S)-piperidin-3-yl]-1H-pyrazole-5-carboxamide hydrochloride (cpd 81);
N-(2-aminocyclohexyl)-3-{2-[(3,5-dimethylphenyl)amino]pyrimidin-4-yl}-1-methyl-1H-pyrazole-5-carboxamide hydrochloride (cpd 82);
3-{2-[(3,5-dimethylphenyl)amino]pyrimidin-4-yl}-1-methyl-N-[2-(pyrrolidin-1-yl)ethyl]-1H-pyrazole-5-carboxamide (cpd 83);
3-{2-[(3,5-dimethylphenyl)amino]pyrimidin-4-yl}-1-methyl-N-[2-(piperidin-1-yl)ethyl]-1H-pyrazole-5-carboxamide (cpd 84);
(3-{2-[(3,5-dimethylphenyl)amino]pyrimidin-4-yl}-1-methyl-1H-pyrazol-5-yl)[(3R)-3-hydroxypyrrolidin-1-yl]methanone (cpd 85);
[(3S)-3-(dimethylamino)pyrrolidin-1-yl](3-{2-[(3,5-dimethylphenyl)amino]pyrimidin-4-yl}-1-methyl-1H-pyrazol-5-yl)methanone (cpd 86);
[(3R)-3-(dimethylamino)pyrrolidin-1-yl](3-{2-[(3,5-dimethylphenyl)amino]pyrimidin-4-yl}-1-methyl-1H-pyrazol-5-yl)methanone (cpd 87);
(3-aminopyrrolidin-1-yl)(3-{2-[(3,5-dimethylphenyl)amino]pyrimidin-4-yl}-1-methyl-1H-pyrazol-5-yl)methanone (cpd 88);
(3-{2-[(3,5-dimethylphenyl)amino]pyrimidin-4-yl}-1-methyl-1H-pyrazol-5-yl)[(2S)-2-(hydroxymethyl)pyrrolidin-1-yl]methanone (cpd 89);
(3-{2-[(3,5-dimethylphenyl)amino]pyrimidin-4-yl}-1-methyl-1H-pyrazol-5-yl)[(3S)-3-hydroxypyrrolidin-1-yl]methanone (cpd 90);
3-{2-[(3,5-dimethylphenyl)amino]pyrimidin-4-yl}-N-[(1R,2S)-2-hydroxycyclohexyl]-1-methyl-1H-pyrazole-5-carboxamide (cpd 91);
N-[(1S,2R)-2-aminocyclohexyl]-3-{2-[(3,5-dimethylphenyl)amino]pyrimidin-4-yl}-1-methyl-1H-pyrazole-5-carboxamide hydrochloride (cpd 92);
N-[(2S)-1-hydroxypropan-2-yl]-1-methyl-3-{2-[(1,2,3-trimethyl-1H-indol-5-yl)amino]pyrimidin-4-yl}-1H-pyrazole-5-carboxamide (cpd 93);
3-{2-[(3,5-dimethylphenyl)amino]pyrimidin-4-yl}-1-methyl-N-[(2S)-1-(pyrrolidin-1-yl)propan-2-yl]-1H-pyrazole-5-carboxamide (cpd 94);
N-[(2S)-1-(azetidin-1-yl)propan-2-yl]-3-{2-[(3,5-dimethylphenyl)amino]pyrimidin-4-yl}-1-methyl-1H-pyrazole-5-carboxamide (cpd 95);
(1R,4S)-2,5-diazabicyclo[2.2.1]hept-2-yl(3-{2-[(3,5-dimethylphenyl)amino]pyrimidin-4-yl}-1-methyl-1H-pyrazol-5-yl)methanone hydrochloride (cpd 96);
3-{2-[(3,5-dimethylphenyl)amino]pyrimidin-4-yl}-1-methyl-N-[(3R)-piperidin-3-yl]-1H-pyrazole-5-carboxamide hydrochloride (cpd 97);
N-[(1R,2S)-2-aminocyclohexyl]-3-{2-[(3,5-dimethylphenyl)amino]pyrimidin-4-yl}-1-methyl-1H-pyrazole-5-carboxamide hydrochloride (cpd 98);
N-[(1S,2R)-2-aminocyclohexyl]-3-{2-[(3,5-dichlorophenyl)amino]pyrimidin-4-yl}-1-methyl-1H-pyrazole-5-carboxamide hydrochloride (cpd 99);
N-[(1S,2R)-2-aminocyclohexyl]-3-{2-[(3,5-dimethoxyphenyl)amino]pyrimidin-4-yl}-1-methyl-1H-pyrazole-5-carboxamide hydrochloride (cpd 100);
N-[(1S,2S)-2-aminocyclohexyl]-3-{2-[(3,5-dimethylphenyl)amino]pyrimidin-4-yl}-1-methyl-1H-pyrazole-5-carboxamide (cpd 101);
N-[(2S)-1-(azetidin-1-yl)-3-methylbutan-2-yl]-3-{2-[(3,5-dimethylphenyl)amino]pyrimidin-4-yl}-1-methyl-1H-pyrazole-5-carboxamide (cpd 102);
3-{2-[(3,5-dimethylphenyl)amino]pyrimidin-4-yl}-1-methyl-N-[(2S)-3-methyl-1-(pyrrolidin-1-yl)butan-2-yl]-1H-pyrazole-5-carboxamide (cpd 103);
N-[(1S,2R)-2-aminocyclohexyl]-3-{2-[(3,5-dimethylphenyl)amino]pyrimidin-4-yl}-1H-pyrazole-5-carboxamide hydrochloride (cpd 104);
N-[(2S)-1-(azetidin-1-yl)-3-methylbutan-2-yl]-3-{2-[(3,5-dimethylphenyl)amino]pyrimidin-4-yl}-1H-pyrazole-5-carboxamide (cpd 105);
N-[(1S)-1-cyclohexyl-2-hydroxyethyl]-3-{2-[(3,5-dimethylphenyl)amino]pyrimidin-4-yl}-1-methyl-1H-pyrazole-5-carboxamide (cpd 106);
3-{2-[(3,5-dimethylphenyl)amino]pyrimidin-4-yl}-1-methyl-N-[(1S)-1-phenyl-2-(pyrrolidin-1-yl)ethyl]-1H-pyrazole-5-carboxamide (cpd 107);
N-[(2S)-1-(azetidin-1-yl)propan-2-yl]-1-methyl-3-{2-[(3-methylphenyl)amino]pyrimidin-4-yl}-1H-pyrazole-5-carboxamide (cpd 108);
3-{2-[(3,5-dimethylphenyl)amino]pyrimidin-4-yl}-N-[(2S)-1-hydroxy-3-phenylpropan-2-yl]-1-methyl-1H-pyrazole-5-carboxamide (cpd 109);
3-(2-{[3-chloro-4-(4-methylpiperazin-1-yl)phenyl]amino}pyrimidin-4-yl)-N-[(2S)-1-hydroxypropan-2-yl]-1-methyl-1H-pyrazole-5-carboxamide (cpd 110);
3-(2-{[3-chloro-4-(4-methylpiperazin-1-yl)phenyl]amino}pyrimidin-4-yl)-N-[(2S)-1-hydroxy-3-methylbutan-2-yl]-1-methyl-1H-pyrazole-5-carboxamide (cpd 111);
1-methyl-3-{2-[(3-methylphenyl)amino]pyrimidin-4-yl}-N-[(2S)-3-methyl-1-(pyrrolidin-1-yl)butan-2-yl]-1H-pyrazole-5-carboxamide (cpd 112);
N-[(1S,2S)-2-aminocyclohexyl]-3-{2-[(3,5-difluorophenyl)amino]pyrimidin-4-yl}-1-methyl-1H-pyrazole-5-carboxamide (cpd 113);
3-(2-{[3-chloro-4-(4-methylpiperazin-1-yl)phenyl]amino}pyrimidin-4-yl)-N-[(1S,2R)-2-hydroxycyclohexyl]-1-methyl-1H-pyrazole-5-carboxamide (cpd 114);
3-{2-[(3,5-dimethylphenyl)amino]pyrimidin-4-yl}-N-[(1S,2R)-2-hydroxycyclohexyl]-1-methyl-1H-pyrazole-5-carboxamide (cpd 115);
N-[(1S,2S)-2-aminocyclohexyl]-3-{2-[(3,5-dichlorophenyl)amino]pyrimidin-4-yl}-1-methyl-1H-pyrazole-5-carboxamide (cpd 116);
3-{2-[(3,5-dichlorophenyl)amino]pyrimidin-4-yl}-1-methyl-N-[(2S)-3-methyl-1-(pyrrolidin-1-yl)butan-2-yl]-1H-pyrazole-5-carboxamide (cpd 117);
3-{2-[(3,5-dimethylphenyl)amino]pyrimidin-4-yl}-N-[(1S,2S)-2-hydroxycyclohexyl]-1-methyl-1H-pyrazole-5-carboxamide (cpd 118);
N-[(2S)-1-(azetidin-1-yl)propan-2-yl]-3-{2-[(3-cyanophenyl)amino]pyrimidin-4-yl}-1-methyl-1H-pyrazole-5-carboxamide (cpd 119);
3-{2-[(3,5-dimethylphenyl)amino]pyrimidin-4-yl}-1-ethyl-N-[(1S,2R)-2-hydroxycyclohexyl]-1H-pyrazole-5-carboxamide (cpd 120);
N-[(1S,2R)-2-aminocyclohexyl]-3-{2-[(3,5-dimethylphenyl)amino]pyrimidin-4-yl}-1-ethyl-1H-pyrazole-5-carboxamide hydrochloride (cpd 121);
3-{2-[(3,5-dimethylphenyl)amino]pyrimidin-4-yl}-1-ethyl-N-[(3R)-piperidin-3-yl]-1H-pyrazole-5-carboxamide hydrochloride (cpd 122) and
N-[(2S)-1-(azetidin-1-yl)propan-2-yl]-3-{2-[(3,5-dimethoxyphenyl)amino]pyrimidin-4-yl}-N,1-dimethyl-1H-pyrazole-5-carboxamide (cpd 123).

For a reference to any specific compound of formula (I) of the invention, optionally in the form of a pharmaceutically acceptable salt, see the experimental section and claims.

If a stereogenic center or another form of an isomeric center is present in a compound of the present invention, all forms of such isomer or isomers, including enantiomers and diastereomers, are intended to be covered herein. Compounds containing a stereogenic center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention.

In cases wherein compounds may exist in tautomeric forms, such as keto-enol tautomers, each tautomeric form is contemplated as being included within this invention whether existing in equilibrium or predominantly in one form. As such, unless otherwise provided, when in compounds of formula (I) R2 is hydrogen, and only one of the following tautomeric forms of formula (Ia) or (Ib) is indicated, the remaining one has still to be intended as comprised within the scope of the invention:

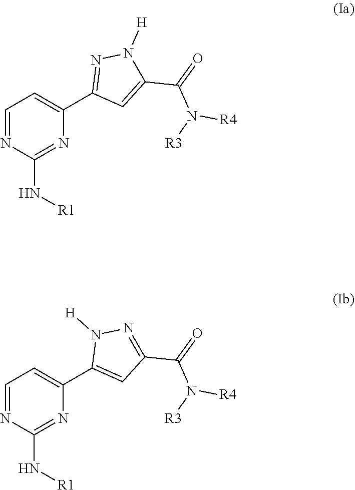

Pharmaceutically acceptable salts of the compounds of formula (I) include the salts with inorganic or organic acids, e.g. nitric, hydrochloric, hydrobromic, sulfuric, perchloric, phosphoric, acetic, trifluoroacetic, propionic, glycolic, lactic, oxalic, fumaric, malonic, malic, maleic, tartaric, citric, benzoic, cinnamic, mandelic, methanesulphonic, isethionic, salicylic, succinic, and p-toluensulphonic acid.

Pharmaceutically acceptable salts of the compounds of formula (I) also include the salts with inorganic or organic bases, e.g. alkali or alkaline-earth metals, especially sodium, potassium, calcium, ammonium or magnesium hydroxides, carbonates or bicarbonates, acyclic or cyclic amines.

Further object of the present invention are compounds of formula (I) as defined above, as well as their isomers, tautomers, hydrates, solvates, complexes, metabolites, prodrugs, carriers and N-oxides.

A metabolite of a compound of formula (I) is any compound into which this same compound of formula (I) is converted in vivo, for instance upon administration to a mammal in need thereof. Typically, without however representing a limiting example, upon administration of a compound of formula (I), this same derivative may be converted into a variety of compounds, for instance including more soluble derivatives like hydroxylated derivatives, which are easily excreted. Hence, depending upon the metabolic pathway thus occurring, any of these hydroxylated derivatives may be regarded as a metabolite of the compounds of formula (I).

Prodrugs are any covalently bound compounds, which release in vivo the active parent drug according to formula (I). N-oxides are compounds of formula (I) wherein nitrogen and oxygen are tethered through a dative bond.

With the term “straight or branched (C₁-C₆) alkyl”, hence comprehensive of (C₁-C₄) alkyl, we intend any of the groups such as, for instance, methyl, ethyl, n-propyl, isopropyl, n-butyl, isobutyl, tert-butyl, sec-butyl, n-pentyl, n-hexyl, and the like.

With the term “straight or branched (C₂-C₆) alkenyl”, we intend any of the groups such as, for instance, vinyl, allyl, 1-propenyl, isopropenyl, 1-butenyl, 2-butenyl, 3-butenyl, 2-pentenyl, 1-hexenyl, and the like.

With the term “straight or branched (C₂-C₆) alkynyl” we intend any of the groups such as, for instance, ethynyl, 2-propynyl, 4-pentynyl, and the like.

With the term “(C₃-C₇) cycloalkyl”, we intend, unless otherwise provided, 3- to 7-membered all-carbon monocyclic ring, which may contain one or more double bonds but does not have a completely conjugated π-electron system. Examples of cycloalkyl groups, without limitation, are cyclopropane, cyclobutane, cyclopentane, cyclopentene, cyclohexane, cyclohexene, cyclohexadiene, cycloheptane, cycloheptene, cycloheptadiene.

The term “aryl” refers to a mono-, bi- or poly-carbocyclic hydrocarbon with from 1 to 4 ring systems, optionally further fused or linked to each other by single bonds, wherein at least one of the carbocyclic rings is “aromatic”, wherein the term “aromatic” refers to completely conjugated π-electron bond system. Non limiting examples of such aryl groups are phenyl, α- or β-naphthyl, α- or β-tetrahydronaphthalenyl, biphenyl, and indanyl groups.

The term “heteroaryl” refers to aromatic heterocyclic rings, typically 5- to 7-membered heterocycles with from 1 to 3 heteroatoms selected among N, O or S; the heteroaryl ring can be optionally further fused or linked to aromatic and non-aromatic carbocyclic and heterocyclic rings. Not limiting examples of such heteroaryl groups are, for instance, pyridyl, pyrazinyl, pyrimidinyl, pyridazinyl, indolyl, imidazolyl, thiazolyl, isothiazolyl, pyrrolyl, furanyl, oxazolyl, isoxazolyl, pyrazolyl, thiophenyl, thiadiazolyl, isoxazolyl, isothiazolyl, oxadiazolyl, indazolyl, cinnolinyl, benzo[1,3]dioxolyl, benzo[1,4]dioxinyl, benzothiazolyl, benzothiophenyl, benzofuranyl, isoindolinyl, benzoimidazolyl, benzoxazolyl, quinolinyl, isoquinolinyl, 1,2,3-triazolyl, 1-phenyl-1,2,3-triazolyl, 2,3-dihydroindolyl, 2,3-dihydrobenzofuranyl, 2,3-dihydrobenzothiophenyl, benzopyranyl, 2,3-dihydrobenzoxazinyl, 2,3-dihydroquinoxalinyl and the like.

With the term “heterocyclyl”, we intend a 3- to 7-membered, saturated or partially unsaturated carbocyclic ring where one or more carbon atoms are replaced by heteroatoms such as nitrogen, oxygen and sulfur. Non limiting examples of heterocyclyl groups are, for instance, pyranyl, tetrahydropyranyl, pyrrolidinyl, pyrrolinyl, imidazolinyl, imidazolidinyl, pyrazolidinyl, pyrazolinyl, thiazolinyl, thiazolidinyl, dihydrofuranyl, tetrahydrofuranyl, tetrahydropyridinyl, 1,3-dioxolanyl, piperidinyl, piperazinyl, morpholinyl and the like. The heterocyclyl ring can be optionally further fused or linked to aromatic and non-aromatic carbocyclic or heterocyclic rings.

According to the present invention and unless otherwise provided, any of the above R1, R2, R3 and R4, groups may be optionally substituted, in any of their free positions, by one or more groups, for instance 1 to 6 groups, independently selected from: halogen, nitro, oxo groups (═O), cyano, (C1-C6) alkyl, polyfluorinated alkyl, polyfluorinated alkoxy, alkenyl, alkynyl, hydroxyalkyl, aryl, arylalkyl, heterocyclyl, (C3-C7) cycloalkyl, hydroxy, alkoxy, aryloxy, heterocyclyloxy, methylenedioxy, alkylcarbonyloxy, arylcarbonyloxy, cycloal kenyloxy, heterocyclylcarbonyloxy, alkylideneaminooxy, carboxy, alkoxycarbonyl, aryloxycarbonyl, cycloalkyloxycarbonyl, heterocyclyloxycarbonyl, amino, ureido, alkylamino, dialkylamino, arylamino, diarylamino, heterocyclylamino, formylamino, alkylcarbonylamino, arylcarbonylamino, heterocyclylcarbonylamino, aminocarbonyl, alkylaminocarbonyl, dialkylaminocarbonyl, arylaminocarbonyl, heterocyclylaminocarbonyl, alkoxycarbonylamino, hydroxyaminocarbonyl alkoxyimino, alkylsulfonylamino, arylsulfonylamino, heterocyclylsulfonylamino, formyl, alkylcarbonyl, arylcarbonyl, cycloalkylcarbonyl, heterocyclylcarbonyl, alkylsulfonyl, arylsulfonyl, aminosulfonyl, alkylaminosulfonyl, dialkylaminosulfonyl, arylaminosulfonyl, heterocyclylaminosulfonyl, arylthio, alkylthio, phosphonate and alkylphosphonate.

In their turn, whenever appropriate, each of the above substituent may be further substituted by one or more of the aforementioned groups.

In this respect, with the term “halogen” we intend a fluorine, chlorine, bromine or iodine atom.

With the term “cyano” we intend a —CN residue.

With the term “nitro” we intend a —NO₂ group.

With the term “alkenyl” or “alkynyl” we intend any of the aforementioned straight or branched (C₂-C₆) alkyl groups further bearing a double or triple bond. Non limiting examples of alkenyl or alkynyl groups of the invention are, for instance, vinyl, allyl, 1-propenyl, isopropenyl, 1-butenyl, 2-butenyl, 3-butenyl, 2-pentenyl, 1-hexenyl, ethynyl, 2-propynyl, 4-pentynyl, and the like.

With the term “polyfluorinated alkyl or alkoxy” we intend any of the above straight or branched (C1-C6) alkyl or alkoxy groups which are substituted by more than one fluorine atom such as, for instance, trifluoromethyl, trifluoroethyl, 1,1,1,3,3,3-hexafluoropropyl, trifluoromethoxy and the like.

With the terms “alkoxy, aryloxy, heterocyclyloxy” and derivatives thereof, we intend any of the above (C1-C6) alkyl, aryl or heterocyclyl groups linked to the rest of the molecule through an oxygen atom (—O—).

From all of the above, it is clear to the skilled person that any group whose name is a composite name such as, for instance, arylamino, has to be intended as conventionally construed by the parts from which it derives, e.g. by an amino group which is further substituted by aryl, wherein aryl is as above defined.

Likewise, any of the terms such as, for instance, alkylthio, alkylamino, dialkylamino, alkoxycarbonyl, alkoxycarbonylamino, heterocyclylcarbonyl, heterocyclylcarbonylamino, cycloalkyloxycarbonyl and the like, include groups wherein the alkyl, alkoxy, aryl, (C₃-C₇) cycloalkyl and heterocyclyl moieties are as above defined.

The present invention also provides a process for the preparation of a compound of formula (I) as defined above, by using the reaction routes and synthetic schemes described below, employing the techniques available in the art and starting materials readily available. The preparation of certain embodiments of the present invention is described in the examples that follow, but those of ordinary skill in the art will recognize that the preparations described may be readily adapted to prepare other embodiments of the present invention. For example, the synthesis of non-exemplified compounds according to the invention may be performed by modifications apparent to those skilled in the art, for instance, by appropriately protecting interfering groups, by changing to other suitable reagents known in the art, or by making routine modifications of reaction conditions. Alternatively, other reactions referred to herein or known in the art will be recognized as having adaptability for preparing other compounds of the invention.

The compounds of this invention can be prepared from readily available starting materials using the following general methods and procedures. Unless otherwise indicated, the starting materials are known compounds or may be prepared from known compounds according to well known procedures. It will be appreciated that, where typical or preferred process conditions (i.e., reaction temperatures, times, mole ratios of reactants, solvents, pressures) are described, different process conditions can also be used unless otherwise stated. Optimum reaction conditions may vary with the particular reactants or solvent used, but such conditions can be determined by one skilled in the art by routine optimization procedures.

Intermediate compounds of formula (V) can be prepared as reported in Scheme 1 below.

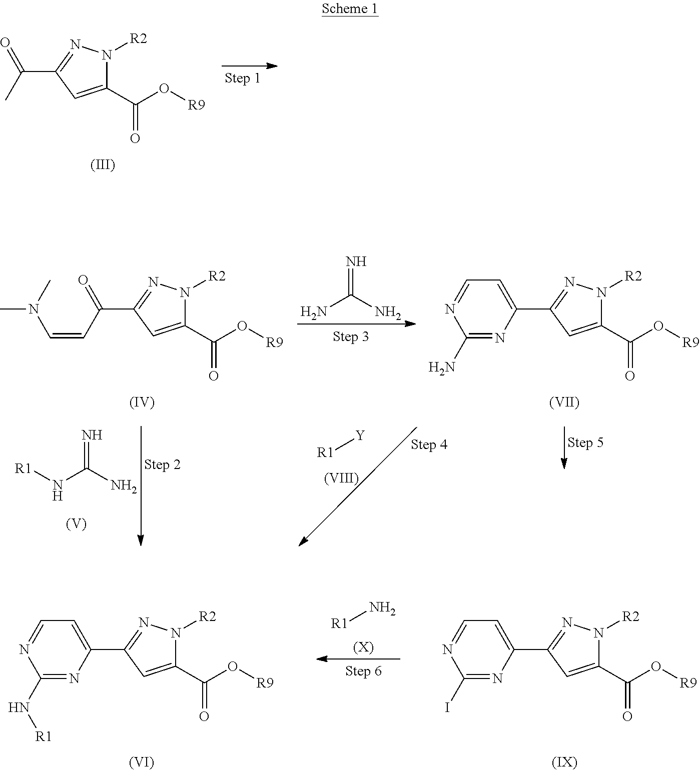

Accordingly, a process of the present invention comprises the following steps:
st. 1) mixing the compound of formula (III):

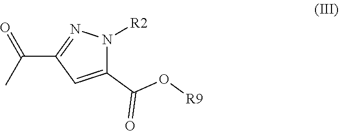

Wherein, R2 is hydrogen or an optionally substituted group selected from straight or branched (C1-C6) alkyl, (C2-C6) alkenyl, (C2-C6) alkynyl, and (C3-C7) cycloalkyl and R9 is a group selected from straight or branched (C1-C6) alky, with a dimethylformamide-dialkylacetale;
st. 2) reacting the resultant compound of formula (IV):

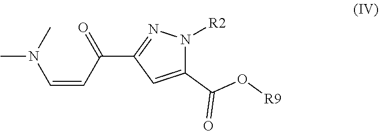

wherein R2 and R9 are as defined above, with a compound of formula (V):

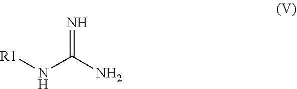

wherein R1 is hydrogen or an optionally substituted group selected from straight or branched (C1-C6) alkyl, (C3-C7) cycloalkyl, aryl, heterocyclyl and heteroaryl, so as to obtain a compound of formula (VI)

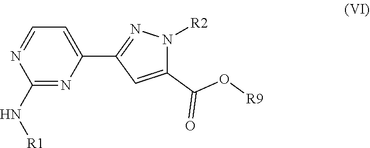

wherein R1, R2 and R9 are as defined as above.

Compound of formula (VI), as reported under step 2, can be alternatively prepared according to the steps below:
st. 3) reacting the formula (IV):

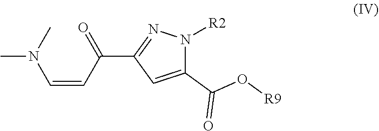

wherein R2 and R9 are as defined above in step 1), with guanidine carbonate;
st. 4) reacting the resultant compound of formula (VII)

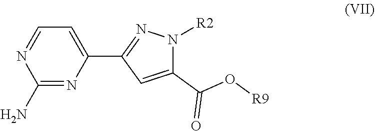

wherein R2 and R9 are as defined above, with a compound of formula (VIII):

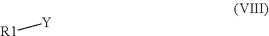

wherein R1 is as defined above under step 2) and Y is iodine or bromine in presence of Palladium, so as to obtain a compound of formula (VI)

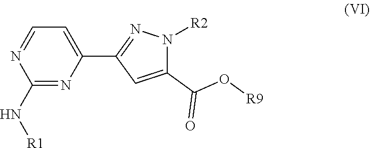

wherein R1, R2 and R9 are as defined as above;
or
st. 5) reacting the resultant compound of formula (VII)

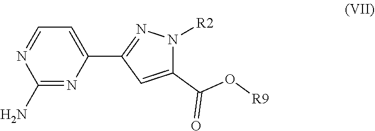

obtained as reported under step 3), by reaction with iso-amylnitrite and diiodomethane or cesium iodide, in the presence of iodine and CuI, thus to obtain a compound of formula (IX) wherein R2 and R9 are as defined above;
st. 6) then, reacting the resultant compound of formula (IX)

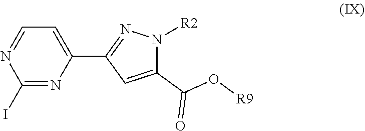

wherein R2 and R9 are as defined above, with a compound of formula (X):

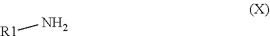

wherein R1 is as defined above, in presence of Palladium, so as to obtain a compound of formula (VI)

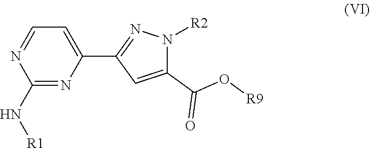

wherein R1, R2 and R9 are as defined as above.

Compound of formula (I) object of the present invention can be prepared as reported in Scheme 2 below:

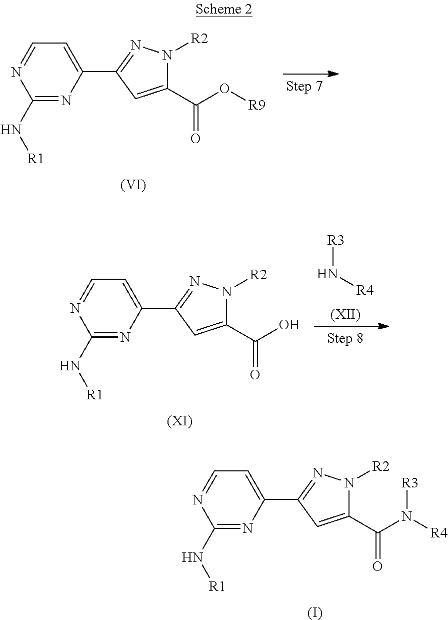

In the above scheme R1, R2, R3 and R4 are as defined in formula (I); R9 is a group selected from straight or branched (C1-C6) alkyl as defined above.

Accordingly, a process of the present invention comprises the following steps:
st. 7) reacting the compound of formula (VI) obtained as described in steps 2, 4 or 6

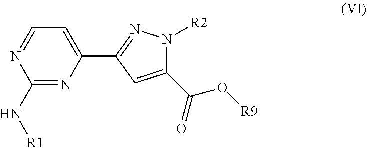

Wherein, R1 is hydrogen or an optionally substituted group selected from straight or branched (C1-C6) alkyl, (C3-C7) cycloalkyl, aryl, heterocyclyl and heteroaryl; R2 is hydrogen or an optionally substituted group selected from straight or branched (C1-C6) alkyl, (C2-C6) alkenyl, (C2-C6) alkynyl, and (C3-C7) cycloalkyl and R9 is a group selected from straight or branched (C1-C6) alky, in acid or basic hydrolysis conditions as to obtain a compound of formula (XI) or the corresponding salt;

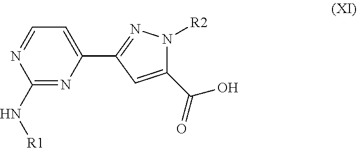

wherein R1 and R2 are as defined as above;
st. 8) reacting the compound of formula (XI) or the corresponding salt as described in steps 7 with compound of formula (XII)

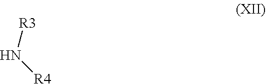

wherein R3 and R4 are independently hydrogen, straight or branched (C1-C6) alkyl optionally substituted with halogens, heteroaryl and heteroaryl (C1-C6) alkyl or a group of formula (11):

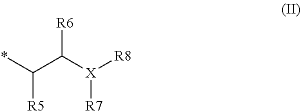

wherein:
R5 is hydrogen, an optionally substituted straight or branched (C1-C6) alkyl, (C3-C7) cycloalkyl, aryl or together with R6 may form an optionally substituted 4- to 7 membered cycloalkyl or, taken together with R7 or R8, may form an optionally substituted heterocyclyl group;
R6 is hydrogen or methyl or together with R3 or R4 may form an optionally substituted 4- to 7 membered heterocyclic group;
R7 and R8 are independently hydrogen, an optionally substituted straight or branched (C1-C6) alkyl or may form together with X an optionally substituted 4- to 7 membered heterocyclyl group optionally containing one additional heteroatom selected from N, O and S or may form together with R3 or R4 an optionally substituted 5- to 7 membered heterocyclyl group;
X is H, N or O, thus to obtain a compound of formula (I)

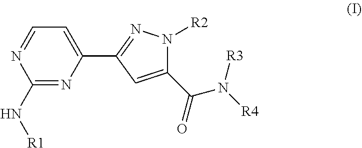

wherein R1, R2, R3 and R4 are as defined above.

According to step 1 of the process, the synthesis of the enaminone derivative of formula (IV) is accomplished using a N,N-dimethylformamide-dialkylacetale, such as, for instance dimethylformamide-di-tert-butylacetale, dimethylformamide-diethylacetale and the like, with or without a suitable solvent such as DMF, DMA, toluene, or the like at a temperature ranging from r.t. to 150° C. by both conventional or microwave heating, and for a time ranging from 30 min to about 24 h.

According to step 2 of the process, the compound of formula (IV) is reacted with a derivative of formula (V) in presence, optionally, of a base selected from AcOK, EtONa, TEA, K₂CO₃ or Na₂CO₃ in a suitable solvent such as, for instance, DMF, EtOH or toluene, at a temperature ranging from r.t. to 150° C. by both conventional or microwave heating, and for a time ranging from about 1 to about 48 h. Preferably, the reaction is carried out in EtOH in microwave equipment at 150° C., for 2 h.

According to step 3 of the process, the compound of formula (IV) is reacted with a guanidine, or its salt or a protected synthetic equivalent such as Boc-guanidine, in presence, eventually, of a base selected from AcOK, EtONa, TEA, K₂CO₃ or Na₂CO₃ in a suitable solvent such as, for instance, DMF, EtOH, PrOH, n-BuOH or toluene, at a temperature ranging from r.t. to 150° C. by both conventional or microwave heating, and for a time ranging from about 1 to about 48 h.

According to step 4 of the process, the compound of formula (VII) as defined above is reacted with compounds of formula (VIII) as defined above according to conventional methods well known in the literature. As an example the reaction can be carried out in a suitable solvent such as DMF, DME, dioxane or CH₃CN and in the presence of a Pd₂(dba)₃, BINAP or 2-(dicyclohexylphosphino)-2′,4′,6′-triisopropyl-1,1′-biphenyl (X-phos) and a base such as K₂CO₃, potassium phosphate or Cs₂CO₃, at a temperature ranging from r.t. to 110° C. and for a time ranging from 2 to about 24 h to obtain compound of formula (VI).

According to step 5 of the process, the reaction is carried out using iso-amylnitrite and diiodomethane or cesium iodide, in the presence of iodine and CuI in a suitable solvent such as dioxane, THF, Et₂O or DME, at a temperature ranging from r.t. to about 100° C., and for a time of about 1 h to about 16 h.

According to step 6 of the process, reaction of a compound of formula (IX) with a compound of formula (X), is carried out in a suitable solvent such as dioxane, DMF, DME or CH₃CN and in the presence of catalytic amounts of Pd(OAc)₂, BINAP or Xantphos and a base such as K₂CO₃, potassium phosphate or Cs₂CO₃, at a temperature ranging from r.t. to 110° C. and for a time ranging from about 2 to about 24 h.

According to step 7 of the process, a compound of formula (VI), can be converted into the corresponding derivative of formula (XI) or a salt thereof, through basic or acidic hydrolysis conditions, widely known in the art. Preferably, the reaction is carried out with aqueous alkaline solutions such as aqueous lithium, sodium or potassium hydroxide in the presence of a suitable solvent such as a lower alcohol, THF, DMF or mixtures thereof; preferably the reaction is carried out with lithium hydroxide in THF/MeOH/water mixture, at a temperature ranging from about r.t. to about 80° C. and for a time ranging from about 2 to about 24 h. According to the operative conditions being employed, the compound of formula (XI) could be obtained either in its acidic form or, alternatively, as a salt.

According to step 8 of the process, the amidation of a carboxylic acid of formula (XI) to yield the corresponding compound of formula (I), is carried out in the presence of ammonium chloride or a suitable primary or secondary amine of formula R3R4NH (XII), under basic conditions, preferably with DIPEA or TEA, in a suitable solvent such as DCM, DMF, THF, 1,4-dioxane, or DMA, in the presence of a suitable condensing agent, for instance dicyclohexylcarbodiimide (DCC), 1-ethyl-3-(3′-dimethylaminopropyl)carbodiimide (EDC), 3,4-dihydro-3-hydroxy-4-oxo-1,2,3-benzotriazine (DHBT), O-benzotriazolyltetramethylisouronium tetrafluoroborate (TBTU), benzotriazol-1-yloxytripyrrolidinophosphonium hexafluorophosphate (PyBOP), or 2-(1H-benzotriazole-1-yl)-1,1,3,3-tetramethyluronium hexafluorophosphate (HBTU). The said reaction is optionally carried out in the presence of a suitable catalyst such as the 4-dimethylaminopyridine, or in the presence of a further coupling reagent such as N-hydroxybenzotriazole (HOBt) at r.t. for a time ranging from about 2 to about 24 h.

From all of the above, it is clear to the skilled person that the conversion of a compound of formula (I) into a pharmaceutically acceptable salt thereof or, alternatively, the conversion into the free compound (I) of a corresponding salt, according to procedures well-known in the art, is still within the scope of the invention. When preparing the compounds of formula (I) according to any variant of the process, which are all to be intended as within the scope of the invention, optional functional groups within the starting materials, the reagents or the intermediates thereof, and which could give rise to unwanted side reactions, need to be properly protected according to conventional techniques.

Protection of such reactive centers, and subsequent deprotection at the end of the synthetic transformations, can be accomplished following standard procedures described, for instance, in: Green, Theodora W. and Wuts, Peter G. M.—Protective Groups in Organic Synthesis, Third Edition, John Wiley & Sons Inc., New York (NY), 1999.

According to any variant of the process for preparing the compounds of the formula (I), the starting materials and any other reactants are known or easily prepared according to known methods.

The compounds of the formula (III) can be prepared as described in WO 2012/139930.

The compounds of the formula (V) are either commercially available or can be prepared with known methods (J. Med. Chem., 2004, vol 47, p. 4716-4730).

The compounds of the formula (VIII), (X) and (XII) are either commercially available or can be prepared with known methods.

The final compounds may be isolated and purified using conventional procedures, for example chromatography and/or crystallization and salt formation.

The compounds of general formula (I) as defined above can be converted into pharmaceutically acceptable salts.

The synthesis of a compound of general formula (I), according to the synthetic processes described above, can be conducted in a stepwise manner, whereby each intermediate is isolated and purified if needed by standard purification techniques, like, for example, column chromatography, before carrying out the subsequent reaction.

Alternatively, two or more steps of the synthetic sequence can be carried out in a so-called “one-pot” procedure, as known in the art, whereby only the compound resultant from the two or more steps is isolated and purified.

In cases where a compound of general formula (I) contains one or more asymmetric centers, said compound can be separated into the single stereoisomers by procedures known to those skilled in the art. Such procedures comprise standard chromatographic techniques, including chromatography using a chiral stationary phase, or crystallization.

General methods for separation of compounds containing one or more asymmetric centers are reported, for instance, in Jacques, Jean; Collet, Andre; Wilen, Samuel H., Enantiomers, Racemates, and Resolutions, John Wiley & Sons Inc., New York (NY), 1981.

The present invention also provides a method for treating diseases caused by and/or associated with dysregulated protein kinase activity, particularly ABL, ACK1, AKT1, ALK, AUR1, AUR2, BRK, BUB1, CDC7/DBF4, CDK2/CYCA, CHK1, CK2, EEF2K, EGFR1, EphA2, EphB4, ERK2, FAK, FGFR1, FLT3, GSK3beta, Haspin, IGFR1, IKK2, IR, JAK1, JAK2, JAK3, KIT, LCK, LYN, MAPKAPK2, MELK, MET, MNK2, MPS1, MST4, NEK6, NIM1, P38alpha, PAK4, PDGFR, PDK1, PERK, PIM1, PIM2, PIM3, PKAalpha, PKCbeta, PLK1, RET, ROS1, SULU1, Syk, TLK2, TRKA, TYK, VEGFR2, VEGFR3, ZAP70; more particularly PIM1, PIM2, PIM3, MPS1, JAK2, JAK3, more particularly Syk family kinases, which comprises administering to a mammal in need thereof, more particularly a human, an effective amount of compound of formula (I) as defined above.

Furthermore the present invention provides a compound of formula (I) or a pharmaceutically acceptable salt thereof, as defined above, for use in a method of treating a disease caused by and/or associated with dysregulated protein kinase activity reported above, particularly Syk kinase activity, which comprises administering to a mammal, preferably a human, in need thereof, an effective amount of a compound of formula (I) as defined above.

A preferred method of the present invention is to treat a disease caused by and/or associated with dysregulated protein kinase activity selected from the group consisting of cancer, cell proliferative disorders, viral infections, immune disorders, neurodegenerative disorders and cardiovascular diseases. More preferably, the disease is cancer.

According to a most preferred embodiment of the present invention the cancer is selected from the group consisting of:
carcinomas, such as bladder, breast, kidney, liver, colon, lung, including small cell lung cancer, esophagus, gall-bladder, ovary, pancreas, stomach, cervix, prostate, and skin, including squamous cell carcinoma; hematopoietic tumors of lymphoid lineage including leukemia, acute lymphocitic leukemia, acute lymphoblastic leukemia, chronic lymphocytic leukemia, B-cell lymphoma, angioimmunoblastic T-cell lymphoma, Hodgkin's lymphoma, non-Hodgkin's lymphoma, hairy cell lymphoma and Burkitt's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including glioma, glioblastoma, glioblastoma multiforme, astrocytoma, oligodendroglioma, paraglioma, neuroblastoma, and schwannomas; and other tumors, including melanoma, seminoma, teratocarcinoma, osteosarcoma, xeroderma pigmentosum, keratoxanthoma, thyroid cancers, such as papillary thyroid carcinoma and medullary thyroid carcinoma, Kaposi's sarcoma, chondrosarcoma, and cholangiocarcinoma.

Another preferred method of the present invention is to treat specific cellular proliferation disorders such as, for example, benign prostate hyperplasia, familial adenomatosis polyposis, neurofibromatosis, psoriasis, vascular smooth cell proliferation associated with atherosclerosis, pulmonary fibrosis, arthritis, glomerulonephritis and postsurgical stenosis and restenosis.

Another preferred method of the present invention is to treat viral infections, in particular the prevention of AIDS development in HIV-infected individuals.

Another preferred method of the present invention is to treat immune disorders, such as inflammatory and autoimmune diseases, for examples multiple sclerosis, rheumatoid arthritis (RA), systemic lupus erythematous, inflammatory bowel diseases (IBD), Crohn's disease, irritable bowel syndrome, pancreatitis, ulcerative colitis, diverticulosis, myasthenia gravis, vasculitis, psoriasis, scleroderma, asthma, allergy, systemic sclerosis, vitiligo, arthritis such as osteoarthritis, juvenile rheumatoid arthritis, ankylosing spondylitis.

Another preferred method of the present invention is to treat neurodegenerative disorders, such as Alzheimer's disease, Parkinson's disease and Huntington's disease.

Another preferred method of the present invention is to treat specific cardiovascular diseases, such as coronary heart diseases, cardiomyopathies, ischaemic heart diseases, heart failure, hypertensive heart diseases, inflammatory heart diseases and valvular heart diseases.

In addition, the method of the present invention also provides tumor angiogenesis and metastasis inhibition as well as the treatment of organ transplant rejection and host versus graft disease.

The present invention also provides a pharmaceutical composition comprising a therapeutically effective amount of a compound of formula (I) or a pharmaceutical acceptable salt thereof and at least one pharmaceutically acceptable excipient, carrier and/or diluent.

The present invention further provides a pharmaceutical composition comprising a compound of formula (I) in combination with one or more chemotherapeutic—e.g. cytostatic or cytotoxic agents. Cytostatic or cytotoxic agents include, but are not limited to antibiotic-type agents, alkylating agents, antimetabolite agents, hormonal agents, immunological agents, interferon-type agents, cyclooxygenase inhibitors (e.g. COX-2 inhibitors), matrixmetalloprotease inhibitors, telomerase inhibitors, tyrosine kinase inhibitors, anti-growth factor receptor agents, anti-HER agents, anti-EGFR agents, anti-angiogenesis agents (e.g. angiogenesis inhibitors), farnesyl transferase inhibitors, ras-raf signal transduction pathway inhibitors, cell cycle inhibitors, other cdks inhibitors, tubulin binding agents, topoisomerase I inhibitors, topoisomerase II inhibitors aromatase inhibitors, inhibitors of kinesins, therapeutic monoclonal antibodies, inhibitors of mTOR, histone deacetylase inhibitors, inhibitors of hypoxic response, PD-1 antagonists, or antigen binding fragment thereof, which specifically binds to PD-1 or PD-L1 and the like.

If formulated as a fixed dose, such combination products employ the compounds of this invention within the dosage range described below and the other pharmaceutically active agent within the approved dosage range.

The present invention further provides an in vitro method for inhibiting Syk protein kinase activity which comprises contacting the Syk kinase with an effective amount of a compound of formula (I) as defined above.

Additionally, the invention provides a product comprising a compound of formula (I) or a pharmaceutically acceptable salt thereof, as defined above, and one or more chemotherapeutic agents, as a combined preparation for simultaneous, separate or sequential use in anticancer therapy.

The compounds of formula (I) of the present invention, suitable for administration to a mammal, e.g. to humans, can be administered by the usual routes and the dosage level depends upon the age, weight, and conditions of the patient and administration route.

For example, a suitable dosage adopted for oral administration of a compound of formula (I) may range from about 10 to about 1000 mg per dose, from 1 to 5 times daily. The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or through intravenous and/or intrathecal and/or intraspinal injection or infusion.

The pharmaceutical compositions containing the compounds of the invention are usually prepared following conventional methods and are administered in a suitable pharmaceutical form.

For example, the solid oral forms may contain, together with the active compound, diluents, e.g. lactose, dextrose, saccharose, sucrose, cellulose, corn starch or potato starch; lubricants, e.g. silica, talc, stearic acid, magnesium or calcium stearate, and/or polyethylene glycols; binding agents, e.g. starches, arabic gum, gelatine methylcellulose, carboxymethylcellulose or polyvinyl pyrrolidone; disintegrating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. These pharmaceutical preparations may be manufactured in known manner, for example, by means of mixing, granulating, tabletting, sugar-coating, or film-coating processes.

The liquid dispersions for oral administration may be, e.g. syrups, emulsions and suspensions.

As an example the syrups may contain, as a carrier, saccharose or saccharose with glycerine and/or mannitol and sorbitol.

The suspensions and the emulsions may contain, as examples of carriers, natural gum, agar, sodium alginate, pectin, methylcellulose, carboxymethylcellulose or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol and, if desired, a suitable amount of lidocaine hydrochloride.

The solutions for intravenous injections or infusions may contain, as a carrier, sterile water or preferably they may be in the form of sterile, aqueous, isotonic, saline solutions or they may contain propylene glycol as a carrier.

The suppositories may contain, together with the active compound, a pharmaceutically acceptable carrier, e.g. cocoa buffer, polyethylene glycol, a polyoxyethylene sorbitan fatty acid ester surfactant or lecithin. In another aspect the invention provides a compound of formula (I) or a pharmaceutically acceptable salt thereof, as defined above, for use as a medicament.

Finally, the invention provides the use of a compound of formula (I) or a pharmaceutically acceptable salt thereof, as defined above, in the manufacture of a medicament with anticancer activity.

### EXPERIMENTAL SECTION

The short forms and abbreviations used herein, as well as throughout the description, have the following meaning:

ABBREVIATIONS

| | |
|---|---|
| AcOEt | ethyl acetate |
| BSA | Bovine serum albumin |
| BINAP | 2,2′-bis(diphenylphosphino)-1,1′-binaphthyl |
| CH₃CN | Acetonitrile |
| DCC | N,N′-dicyclohexylcarbodiimide |
| DCM | Dichloromethane |
| DIPEA | N,N-diisopropyethylamine |
| DMA | N,N-dimethylacetamide |
| DME | 1,2-dimethoxyethane |
| DMF | N,N-dimethylformamide |
| DMSO | dimethyl sulfoxide |
| DTT | Dithiothreitol |
| EDCI | N-ethyl-N′,N-diisopropyl carbodiimide hydrochloride |
| Et₂O | diethyl ether |
| EtOH | Ethanol |
| HOBt | 1H-benzotriazol-1-ol |
| iPrOH | Propan-2-ol[ |
| MeOH | Methanol |
| MTBE | Methyl tert-butyl ether |
| NaOH | sodium hydroxide |
| Pd(OAc)₂ | palladium(II) acetate |
| Pd₂(dba)₃ | tris(dibenzylideneacetone)dipalladium(0) |
| PdCl₂(dppf) | 1,1′-bis(diphenylphosphino)ferrocene]palladium(II) chloride |
| PyBOP | (Benzotriazol-1-yloxy)tripyrrolidinophosphonium |
| | hexafluorophosphate |
| TBTU | O-(benzotriazol-1-yl)-N,N,N′,N′-tetramethyluronium |
| | tetrafluoroborate |
| TEA | Triethylamine |
| THF | Tetrahydrofuran |
| Xantphos | 4,5-bis(diphenylphosphino)-9,9-dimethylxanthene |
| XPhos | 2-Dicyclohexylphosphino-2′,4′,6′-triisopropylbiphenyl |
| H | hour/s |
| Min | minute/s |
| r.t. | room temperature |
| Rt | retention time |

### Preparation of Compounds of Formula (I)

For a reference to any specific compound of formula (I) of the invention, optionally in the form of a pharmaceutically acceptable salt, see the experimental section and claims. Referring to the examples that follow, compounds of the present invention were synthesized using the methods described herein, or other methods, which are well known in the art.

With the aim at better illustrating the present invention, without posing any limitation to it, the following examples are given.

As used herein the symbols and conventions used in the processes, schemes and examples are consistent with those used in the contemporary scientific literature, for example, the Journal of the American Chemical Society or the Journal of Biological Chemistry.

Compound names are IUPAC names, generated by using ACD Name (by Advanced Chemistry Development, Inc.). Unless otherwise noted, all materials, including anhydrous solvent such as DMF, THF, DCM, were obtained from commercial suppliers, of the best grade and used without further purification. All reactions involving air- or moisture-sensitive compounds were performed under nitrogen or argon atmosphere.

### General Purification and Analytical Methods

The synthetic preparation of some compounds of formula (I) of the invention is described in the following examples. The compounds of the present invention, as prepared according to the following examples, were also characterized by ¹H NMR and/or by HPLC/MS analytical data; HPLC/MS data were collected following any one of methods LCQ or LCT. Flash Chromatography was performed on silica gel (Merck grade 9395, 60A).

### HPLC LCQ Method

HPLC-MS/UV analyses were performed on a LCQ DecaXP (Thermo, San Jose, US) ion trap instrument, equipped with an electrospray (ESI) ion source. The mass spectrometer is connected to a Surveyor HPLC system (Thermo, San Jose, US) with an UV photodiode array detector (UV detection 215-400 nm). A Waters XSelect CSH C18 column 50×4.6 mm, 3.5 m particle size was used. Mobile phase A was ammonium acetate 5 mM buffer (pH 4.5 with acetic acid):acetonitrile 95:5, and mobile phase B was ammonium acetate 5 mM buffer (pH 4.5 with acetic acid): acetonitrile 5:95. Gradient from 0 to 100% B in 7 minutes, hold 100% B 2 minutes. Flow rate 1 mL/min. Injection volume 10 μL. Retention times (HPLC r.t.) are given in minutes. Full scan, mass range from 50 to 1200 amu. Heated capillary temp was 200° C. and Spray voltage value was set at 4 kV. Mass are given as m/z ratio.

Instrument control, data acquisition and processing were performed by using Xcalibur 1.4 SR1 software (Thermo).

### HPLC LCT Method

HPLC-MS/UV analyses and High Resolution Mass Spectra (HRMS) were performed on a A Waters Alliance LC 2795 equipped with a Waters PDA UV detector 2996 and a TOF Waters LCT Premier XE mass detector (ESI interface) supported by a Waters Reagent Manager liquid pump. The assay is based on generic gradient reversed-phase chromatography that allows complementing an identity-purity assay with determination and confirmation of the expected exact mass of the compounds in the same run. Compound identity is accomplished by on-line serial ESI(+) Full Scan MS detection, sample purity is obtained as relative “Area Percent” of the integrated LC/UV trace at 216-400 nm. The liquid chromatograph is equipped with a Waters XBridge CSH C18 column (3.0×30 mm, 3.5 m particle size) thermostated at 50° C. Alternatively a Supelco column Ascentis Express C18 (2.7×30 mm×3 um) was used.

Mobile phases A was 0.05% w/v formic acid in highly purified water and mobile phase B was a 70/25/5 (v/v/v) mixture of MeOH/iPrOH/H2O containing 0.035% w/v of formic acid. Gradient from 0 to 100% B in 17.5 minutes, hold 100% B 5 minutes. Flow rate 0.8 mL/min, Injection volume 4 μL. The ESI source operated at 100° C., 2.5 kV capillary voltage, 60 V cone, 600 L/hr nitrogen desolvation flow at 350° C. and 10 L/hr nitrogen cone flow. The “Normal” Zfocus is set at 140. The analyzer is normally optimized at 7200 V flight tube.

In order to obtain high resolution mass spectra, the eluent from the HPLC column was split and 25 L/min were mixed with a 100 L/min stream of a 30/10/60 (v/v/v) mixture of MeOH/iPrOH/H2O containing 0.01% w/v of formic acid and 80 nM Trimethoprim coming from a Waters Reagent Manager pump before entering the MS source. Trimethoprim was chosen as stable, soluble and appropriate reference compound for real-time single-point mass correction. ES(+) full scan 80-1200 amu centroided data acquisition was carried out at 2 Hz sampling rate in the “W” mode. The LCT embedded PC provided both real-time data centroiding and real-time mass correction based on the Trimethoprim. H+ reference mass of 291.1452 Da. Proper intensity MS spectra (40 to 2000 analyte counts) were averaged to obtain the final result.

¹H-NMR spectra were recorded at a constant temperature of 28° C. on a Varian INOVA 400 spectrometer operating at 400.5 MHz and equipped with a 5 mm 1H{¹⁵N-³¹P} z-axis PFG Indirect Detection probe and on a Varian INOVA 500 spectrometer operating at 499.7 MHz and equipped with a 5 mm ¹H{¹³C-¹⁵N} triple resonance Indirect Detection probe. Chemical shifts were referenced with non deuterated residual solvent signal (DMSO-d₅: 2.50 ppm for ¹H). Data are reported as follows: chemical shift (6), multiplicity (s=singlet, d=doublet, t=triplet, q=quartet, qt=quintet, br. s=broad singlet, dd=doublet of doublets, ddd=doublet of doublets of doublets, m=multiplet), coupling constants (J, Hz) and number of protons.

### Examples

### Step 1

### 5-((E)-3-Dimethylamino-acryloyl)-2-methyl-2H-pyrazole-3-carboxylic acid ethyl ester [(IV), R2=methyl, R9=ethyl]

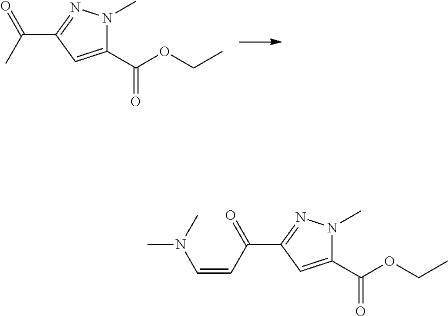 [SMILES: CCOC(=O)c1cc(C(C)=O)nn1C ; CCOC(=O)c1cc(C(=O)/C=C\N(C)C)nn1C]

To 5-Acetyl-2-methyl-2H-pyrazole-3-carboxylic acid ethyl ester (2 g, 10.19 mmol, 1 eq.) in a CEM reactor N,N-dimethylformamide diethyl acetal (10.48 ml, 61.2 mmol, 6 eq.) was added. The mixture was stirred for 3 hours in a CEM explorer Microwave™ at 140° C. Volatiles were removed under vacuum and the solid was rinsed with hexane and filtered affording 2.45 g of the title compound as dark yellow powder in 95% yield.

LC/MS (254 nm) HPLC LCT-Formic Acid Rt 6.73 min.

¹H NMR (500 MHz, DMSO-d₆) δ ppm 7.72 (d, J=12.5 Hz, 1H), 7.12 (s, 1H), 5.83 (d, J=12.7 Hz, 1H), 4.30 (q, J=7.1 Hz, 2H), 4.13 (s, 3H), 3.14 (s, 3H), 2.87 (s, 3H), 1.31 (t, J=7.1 Hz, 3H). HRMS (ESI) calcd for C12H18N3O3 [M+H]⁺ 252.1343 found 252.1342.

According to this same methodology, but employing suitable substituted derivatives, the following compounds were prepared:
5-((Z)-3-Dimethylamino-acryloyl)-2H-pyrazole-3-carboxylic acid ethyl ester [(IV), R2=H, R9=ethyl]
5-((Z)-3-Dimethylamino-acryloyl)-2-ethyl-2H-pyrazole-3-carboxylic acid ethyl ester [(IV), R2=ethyl, R9=ethyl]

### Step 2 and Step 3

### 5-(2-Amino-pyrimidin-4-yl)-2-methyl-2H-pyrazole-3-carboxylic acid ethyl ester [(VII), R2=methyl, R9=ethyl]

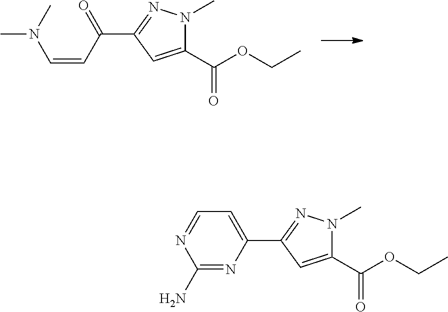 [SMILES: CCOC(=O)c1cc(C(=O)/C=C\N(C)C)nn1C ; CCOC(=O)c1cc(-c2ccnc(N)n2)nn1C]

5-((E)-3-Dimethylamino-acryloyl)-2-methyl-2H-pyrazole-3-carboxylic acid ethyl ester (7.75 g, 30.84 mmol, 1 eq.) and guanidine carbonate (8.34 g, 46.26 mmol, 1.5 eq) were loaded in CEM microwave reactors (divided in six batches) and suspended in ethanol (100 ml, 0.3M). The mixture was stirred for 2 hours in a CEM explorer microwave at 150° C. The six batches were collected and dried. The residue was suspended in water (50 ml) and treated a 0° C. with HCl 1N until pH=2, the suspension was stirred at 0° for 1 hour then filtered and the solid was washed with water and dried. The 7.6 g of crude material (the title compound and its corresponding acid) were dissolved in ethanol (115 ml) and sulfuric acid 96% (21 ml, 378 mmol, 12 eq.) was added. The mixture was heated at reflux for 7 h, half of volatiles were removed under vacuum, the mixture was diluted with water (200 ml) and ethyl acetate (100 ml). The pH was adjusted at 9 under stirring with NaOH conc., the layers were separated and the aqueous phase was extracted with AcOEt (3×50 mL). The combined organic layers were washed with brine then dried over sodium sulfate and, after removal of the solvent, a solid was obtained, which was triturated with di-isopropyl ether to obtain the title compound as a pale yellow solid (6.38 g, 58%).

LC/MS (254 nm) HPLC method (IP) LCQ Deca XP-Acetate Buffer Rt 4.43 min.

¹H NMR (500 MHz, DMSO-d6) δ ppm 8.28 (d, J=5.2 Hz, 1H), 7.32 (s, 1H), 7.06 (d, J=5.2 Hz, 1H), 6.67 (s, 2H), 4.32 (q, J=7.1 Hz, 2H), 4.19-4.12 (m, 3H), 1.33 (t, J=7.1 Hz, 3H); HRMS (ESI) calcd for C11H14N5O2 [M+H]+ 248.1142, found 248.1139.

According to this same methodology, but employing suitable derivatives and the proper guanidine derivative, the following compounds were prepared:

### 5-(2-Amino-pyrimidin-4-yl)-2H-pyrazole-3-carboxylic acid ethyl ester [(VII), R2=H, R9=ethyl]

### 5-(2-Amino-pyrimidin-4-yl)-2-ethyl-2H-pyrazole-3-carboxylic acid ethyl ester [(VII), R2=ethyl, R9=ethyl]

### 5-[2-(3,5-Dimethyl-phenylamino)-pyrimidin-4-yl]-2-methyl-2H-pyrazole-3-carboxylic acid ethyl ester [(VI), R1=3,5-Dimethyl-phenyl, R2=methyl, R9=ethyl]

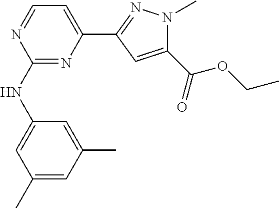 [SMILES: CCOC(=O)c1cc(-c2ccnc(Nc3cc(C)cc(C)c3)n2)nn1C]

LC/MS (254 nm) HPLC method (IP) LCQ Deca XP-Acetate Buffer Rt 7.56 min.

¹H NMR (500 MHz, DMSO-d6) δ ppm 9.52 (s, 1H), 8.52 (d, J=5.1 Hz, 1H), 7.49 (s, 2H), 7.44 (s, 1H), 7.28 (d, J=5.1 Hz, 1H), 6.62 (s, 1H), 4.34 (q, J=7.1 Hz, 2H), 4.19 (s, 3H), 2.27 (s, 6H), 1.34 (t, J=7.1 Hz, 3H); HRMS (ESI) calcd for C19H22N5O2 [M+H]+ 352.1768, found 352.1765.

### 5-[2-(1,5-Dimethyl-1H-pyrazol-3-ylamino)-pyrimidin-4-yl]-2-methyl-2H-pyrazole-3-carboxylic acid ethyl ester [(VI), R1=1,5-Dimethyl-1H-pyrazol-3-yl, R2=methyl, R9=ethyl]

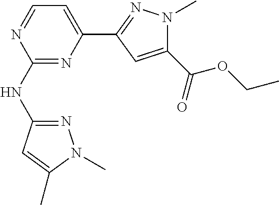 [SMILES: CCOC(=O)c1cc(-c2ccnc(Nc3cc(C)n(C)n3)n2)nn1C]

LC/MS (254 nm) HPLC method (IP) LCT-Formic Acid Rt 9.60 min.

¹H NMR (500 MHz, DMSO-d6) δ ppm 10.17 (s, 1H), 8.45 (d, J=5.0 Hz, 1H), 7.39 (s, 1H), 7.24 (d, J=5.0 Hz, 1H), 6.47 (s, 1H), 4.34 (q, J=7.0 Hz, 2H), 4.18 (s, 3H), 3.67 (s, 3H), 2.23 (s, 3H), 1.34 (t, J=7.2 Hz, 3H); HRMS (ESI) calcd for C16H2ON7O2 [M+H]+ 342.1673, found 342.1674.

### 2-Methyl-5-[2-(1-methyl-1H-pyrazol-4-ylamino)-pyrimidin-4-yl]-2H-pyrazole-3-carboxylic acid ethyl ester [(VI), R1=1-Methyl-1H-pyrazol-4-yl, R2=methyl, R9=ethyl]

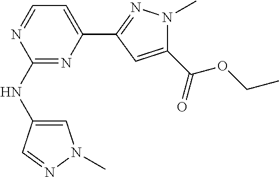 [SMILES: CCOC(=O)c1cc(-c2ccnc(Nc3cnn(C)c3)n2)nn1C]

LC/MS (254 nm) HPLC method (IP) LCT-Formic Acid Rt 9.18 min.

¹H NMR (500 MHz, DMSO-d6) δ ppm 9.52 (s, 1H), 8.46 (d, J=5.0 Hz, 1H), 7.90 (s, 1H), 7.55 (br. s., 1H), 7.42 (s, 1H), 7.20 (d, J=5.0 Hz, 1H), 4.35 (q, J=7.1 Hz, 2H), 4.19 (s, 3H), 3.82 (s, 3H), 1.35 (t, J=7.1 Hz, 3H); HRMS (ESI) calcd for C15H18N7O2 [M+H]+ 328.1517, found 328.1517.

### 2-Methyl-5-[2-(tetrahydro-pyran-4-ylamino)-pyrimidin-4-yl]-2H-pyrazole-3-carboxylic acid ethyl ester [(VI), R1=Tetrahydro-pyran-4-yl, R2=methyl, R9=ethyl]

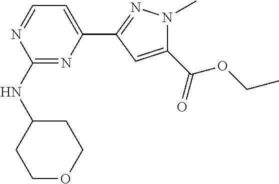 [SMILES: CCOC(=O)c1cc(-c2ccnc(NC3CCOCC3)n2)nn1C]

LC/MS (254 nm) HPLC method (IP) LCT-Formic Acid Rt 8.79 min.

¹H NMR (500 MHz, DMSO-d6) δ ppm 8.32 (d, J=5.0 Hz, 1H), 7.34 (s, 1H), 7.21 (d, J=7.8 Hz, 1H), 7.05 (d, J=5.0 Hz, 1H), 4.36-4.33 (m, 2H), 4.16 (s, 3H), 3.98 (br. s., 2H), 3.92-3.83 (m, 2H), 3.46-3.37 (m, 2H), 1.84 (d, J=11.6 Hz, 2H), 1.59-1.43 (m, 2H), 1.34 (s, 1H); HRMS (ESI) calcd for C16H22N5O3 [M+H]+ 332.1717, found 332.1713.

### 5-[2-(3,5-Dimethoxy-phenylamino)-pyrimidin-4-yl]-2-methyl-2H-pyrazole-3-carboxylic acid ethyl ester [(VI), R1=3,5-Dimethoxy-phenyl, R2=methyl, R9=ethyl]

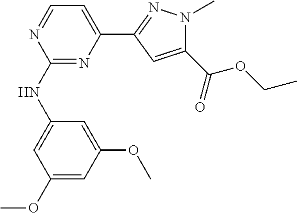 [SMILES: CCOC(=O)c1cc(-c2ccnc(Nc3cc(OC)cc(OC)c3)n2)nn1C]

LC/MS (254 nm) HPLC method (IP) LCO Deca XP-Acetate Buffer Rt 6.84 mi.

¹H NMR (500 MHz, DMSO-d6) δ ppm 9.67 (s, 1H), 8.54 (d, J=5.0 Hz, 1H), 7.49-7.45 (m, 1H), 7.32 (d, J=5.2 Hz, 1H), 7.20 (d, J=2.3 Hz, 2H), 6.14 (t, J=2.2 Hz, 1H), 4.33 (q, J=7.2 Hz, 2H), 4.19 (s, 3H), 3.76 (s, 6H), 1.34 (t, J=7.1 Hz, 3H); HRMS (ESI) calcd for C19H22N5O4 [M+H]+ 384.1667, found 384.1666.

### 5-[2-(3,5-Dimethyl-phenylamino)-pyrimidin-4-yl]-2H-pyrazole-3-carboxylic acid ethyl ester [(VI), R1=3,5-Dimethyl-phenyl, R2=H, R9=ethyl]

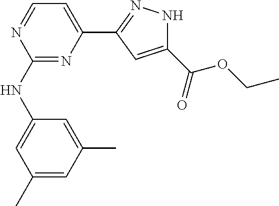 [SMILES: CCOC(=O)c1cc(-c2ccnc(Nc3cc(C)cc(C)c3)n2)n[nH]1]

LC/MS (254 nm) HPLC method (IP) LCQ Deca XP-Acetate Buffer Rt 6.6 min

¹H NMR (500 MHz, DMSO-d6) δ ppm 14.45 (br. s., 1H), 9.52 (s, 1H), 8.54 (br. s., 1H), 7.47 (br. s., 2H), 7.39 (s, 1H), 7.33 (d, J=5.0 Hz, 1H), 6.62 (s, 1H), 4.33 (q, J=6.7 Hz, 2H), 2.26 (s, 6H), 1.33 (t, J=7.1 Hz, 3H); HRMS (ESI) calcd for C18H20N5O2 [M+H]+ 338.1612, found 338.1607.

Alternatively the reaction with substituted guanidine could be performed to obtain directly the corresponding carboxylic acid or its salt.

### 5-[2-(3,5-Dimethoxy-phenylamino)-pyrimidin-4-yl]-2-methyl-2H-pyrazole-3-carboxylic acid [(XI), R1=3,5-Dimethoxy-phenyl, R2=methyl]

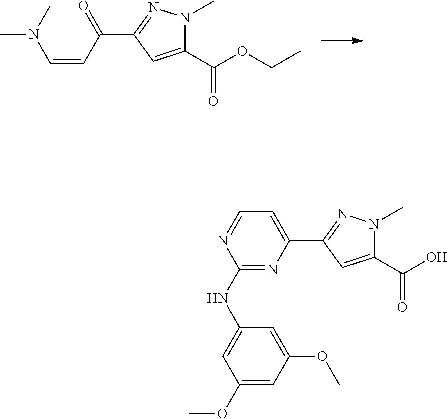

A suspension of 5-((E)-3-Dimethylamino-acryloyl)-2-methyl-2H-pyrazole-3-carboxylic acid ethyl ester (3.64 g, 14.4 mmol), N-(3,5-Dimethoxy-phenyl)-guanidine hydrochloride (4.0 g, 17.3 mmol, 1.2 eq) and potassium carbonate (3.6 g, 25.9 mmol, 1.8 eq) in n-butanol (70 mL) is stirred at 113° C. (internal temperature) for 24 h, after which time HPLC/MS check shows a complete conversion. The reaction mixture is cooled to room temperature, diluted with 70 mL of water and 25 mL of AcOEt and stirred until full dissolution of the suspended solid. The phases are separated and the organic layer extracted 3 times with 30 mL of water. The aqueous phases are combined and brought to acidic pH (ca. 3-4) with 2N HCl (16 mL) under stirring. The resulting suspension is stirred for 10 minutes then filtered over a sintered glass Buchner funnel, porosity 4 (filtration quite slow). The cake is washed on the filter twice with 5 mL of water then twice with 10 mL of MTBE. The solid is collected and dried in oven at 45-50° C. under vacuum (20-30 mm Hg) until constant weight affording 3.23 g of desired product as beige solid. Yield: 63%.

LC/MS (254 nm) HPLC method (IP) LCQ Deca XP-Acetate Buffer Rt 4.13 min

¹H NMR (500 MHz, DMSO-d6) δ ppm 9.67 (s, 1H), 8.56 (d, J=5.0 Hz, 1H), 7.43 (s, 1H), 7.35 (d, J=5.0 Hz, 1H), 7.22 (d, J=2.3 Hz, 2H), 6.17 (t, J=2.3 Hz, 1H), 4.21 (s, 3H), 3.81-3.76 (m, 6H); HRMS (ESI) calcd for C17H18N5O4 [M+H]+ 356.1354, found 356.1366.

### Step 4

### 5-[2-(3,5-Dimethyl-phenylamino)-pyrimidin-4-yl]-2-methyl-2H-pyrazole-3-carboxylic acid ethyl ester [(VI), R1=3,5-Dimethyl-phenyl, R2=methyl, R9=ethyl]

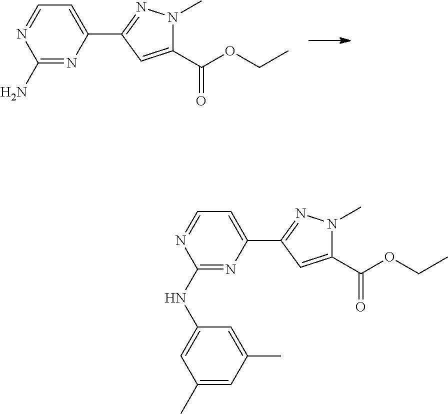 [SMILES: CCOC(=O)c1cc(-c2ccnc(N)n2)nn1C ; CCOC(=O)c1cc(-c2ccnc(Nc3cc(C)cc(C)c3)n2)nn1C]

5-(2-Amino-pyrimidin-4-yl)-2-methyl-2H-pyrazole-3-carboxylic acid ethyl ester (200 mg, 0.81 mmol, 1 eq.), 3,5-dimethyl-iodobenzene (175 ul, 1.21 mmol, 1.5 eq.) and caesium carbonate (527 mg, 1.62 mmol, 2 eq.) were suspended in previously degassed dioxane (8 ml) and three cycles vacuum/argon were performed. XPhos (85 mg, 0.18 mmol, 0.22 eq.) and Tris(dibenzylideneacetone)dipalladium(0) (74 mg, 0.08 mmol, 0.1 eq.) were added under argon atmosphere and three cycles vacuum/argon were performed. The mixture was heated at 100° C. for 6 hours, then cooled to room temperature and diluted with water, extracted three times with AcOEt, the organics were collected and washed with brine and the volatiles were removed under reduced pressure. The crude solid was purified by flash chromatography on silica gel (eluant: DCM:EtOH 99/1) to afford 160 mg (56% yield) of the title compound.

LC/MS (254 nm) HPLC method (IP) LCQ Deca XP-Acetate Buffer Rt 7.56 min.

¹H NMR (500 MHz, DMSO-d6) δ ppm 9.52 (s, 1H), 8.52 (d, J=5.1 Hz, 1H), 7.49 (s, 2H), 7.44 (s, 1H), 7.28 (d, J=5.1 Hz, 1H), 6.62 (s, 1H), 4.34 (q, J=7.1 Hz, 2H), 4.19 (s, 3H), 2.27 (s, 6H), 1.34 (t, J=7.1 Hz, 3H); HRMS (ESI) calcd for C19H22N5O2 [M+H]+ 352.1768, found 352.1765.

According to this same methodology, but employing suitable substituted derivatives, the following compounds were prepared:

### 5-[2-(3,5-Dichloro-phenylamino)-pyrimidin-4-yl]-2-methyl-2H-pyrazole-3-carboxylic acid ethyl ester [(VI), R1=3,5-Dichloro-phenyl, R2=methyl, R9=ethyl] 28% yield

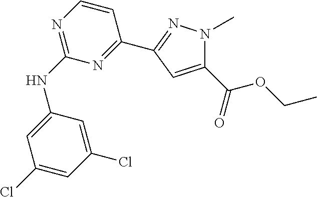 [SMILES: CCOC(=O)c1cc(-c2ccnc(Nc3cc(Cl)cc(Cl)c3)n2)nn1C]

LC/MS (254 nm) HPLC method (IP) LCQ Deca XP-Acetate Buffer Rt 8.21 min.

¹H NMR (500 MHz, DMSO-d6) δ ppm 10.16 (s, 1H), 8.62 (d, J=5.2 Hz, 1H), 8.01 (d, J=1.8 Hz, 2H), 7.47 (s, 1H), 7.42 (d, J=5.2 Hz, 1H), 7.14 (t, J=1.9 Hz, 1H), 4.33 (q, J=7.2 Hz, 2H), 4.20 (s, 3H), 1.33 (t, J=7.1 Hz, 3H); HRMS (ESI) calcd for C17H16Cl2N5O2 [M+H]+ 392.0676, found 392.0675.

### 5-[2-(3,5-Dimethyl-phenylamino)-pyrimidin-4-yl]-2-ethyl-2H-pyrazole-3-carboxylic acid ethyl ester [(VI), R1=3,5-Dimethyl-phenyl, R2=ethyl, R9=ethyl]

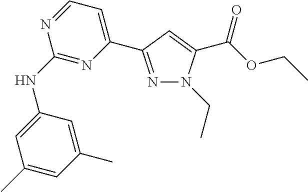 [SMILES: CCOC(=O)c1cc(-c2ccnc(Nc3cc(C)cc(C)c3)n2)nn1CC]

LC/MS (254 nm) HPLC method (IP) LCQ Deca XP-Acetate Buffer Rt 7.18 min

¹H NMR (500 MHz, DMSO-d6) δ ppm 9.51 (s, 1H), 8.55 (d, J=5.2 Hz, 1H), 7.48 (s, 1H), 7.30 (d, J=5.2 Hz, 1H), 7.28 (s, 2H), 6.67 (s, 1H), 4.80 (q, J=7.1 Hz, 2H), 4.30 (q, J=7.1 Hz, 2H), 2.25 (s, 6H), 1.31 (t, J=7.0 Hz, 3H), 1.28 (t, J=7.2 Hz, 3H); HRMS (ESI) calcd for C20H24N5O2 [M+H]+ 366.1925, found 366.1921.

### 2-Methyl-5-(2-m-tolylamino-pyrimidin-4-yl)-2H-pyrazole-3-carboxylic acid ethyl ester [(VI), R1=2-m-tolyl, R2=methyl, R9=ethyl]

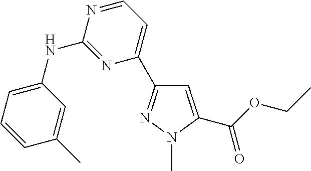 [SMILES: CCOC(=O)c1cc(-c2ccnc(Nc3cccc(C)c3)n2)nn1C]

LC/MS (254 nm) HPLC method (IP) LCQ Deca XP-Acetate Buffer Rt 7.18 min

¹H NMR (500 MHz, DMSO-d6) δ ppm 9.62 (s, 1H), 8.52 (d, J=5.0 Hz, 1H), 7.74 (s, 1H), 7.56 (d, J=8.2 Hz, 1H), 7.44 (s, 1H), 7.30 (d, J=5.0 Hz, 1H), 7.18 (t, J=7.9 Hz, 1H), 6.79 (d, J=7.3 Hz, 1H), 4.34 (q, J=7.0 Hz, 2H), 4.19 (s, 3H), 2.31 (s, 3H), 1.34 (t, J=7.1 Hz, 3H); HRMS (ESI) calcd for C18H20N5O2 [M+H]+ 338.1612, found 338.1615.

### 5-[2-(3,5-Difluoro-phenylamino)-pyrimidin-4-yl]-2-methyl-2H-pyrazole-3-carboxylic acid ethyl ester [(VI), R1=3,5-Difluoro-phenyl, R2=methyl, R9=ethyl]

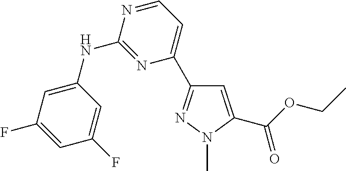 [SMILES: CCOC(=O)c1cc(-c2ccnc(Nc3cc(F)cc(F)c3)n2)nn1C]

LC/MS (254 nm) HPLC method (IP) LCQ Deca XP-Acetate Buffer Rt 7.37 min

¹H NMR (500 MHz, DMSO-d6) δ ppm 10.17 (s, 1H), 8.61 (d, J=5.2 Hz, 1H), 7.67-7.58 (m, 2H), 7.45 (s, 1H), 7.42 (d, J=5.2 Hz, 1H), 6.76 (tt, J=2.3, 9.2 Hz, 1H), 4.34 (q, J=7.0 Hz, 2H), 4.20 (s, 3H), 1.34 (t, J=7.1 Hz, 3H); HRMS (ESI) calcd for C17H16F2N5O2 [M+H]+ 360.1267, found 360.1274.

### 5-{2-[3-Chloro-4-(4-methyl-piperazin-1-yl)-phenylamino]-pyrimidin-4-yl}-2-methyl-2H-pyrazole-3-carboxylic acid ethyl ester [(VI), R1=3-Chloro-4-(4-methyl-piperazin-1-yl)-phenyl, R2=methyl, R9=ethyl]

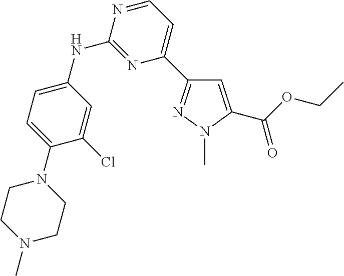 [SMILES: CCOC(=O)c1cc(-c2ccnc(Nc3ccc(N4CCN(C)CC4)c(Cl)c3)n2)nn1C]

LC/MS (254 nm) HPLC method (IP) LCQ Deca XP-Acetate Buffer Rt 5.80 min

¹H NMR (500 MHz, DMSO-d6) δ ppm 9.76 (s, 1H), 8.53 (d, J=5.0 Hz, 1H), 8.16 (br. s., 1H), 7.59 (dd, J=2.5, 8.8 Hz, 1H), 7.45 (s, 1H), 7.31 (d, J=5.0 Hz, 1H), 7.13 (d, J=8.8 Hz, 1H), 4.34 (q, J=7.2 Hz, 2H), 4.19 (s, 3H), 2.93 (br. s., 4H), 2.47 (br. s., 4H), 2.23 (s, 3H), 1.34 (t, J=7.1 Hz, 3H); HRMS (ESI) calcd for C22H27ClN7O2 [M+H]+ 456.1910, found 456.1901.

### 5-[2-(3-Cyano-phenylamino)-pyrimidin-4-yl]-2-methyl-2H-pyrazole-3-carboxylic acid ethyl ester [(VI), R1=3-Cyano-phenyl, R2=methyl, R9=ethyl]

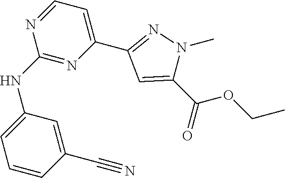 [SMILES: CCOC(=O)c1cc(-c2ccnc(Nc3cccc(C#N)c3)n2)nn1C]

LC/MS (254 nm) HPLC method (IP) LCQ Deca XP-Acetate Buffer Rt 6.73 min

¹H NMR (500 MHz, DMSO-d6) δ ppm 10.12 (s, 1H), 8.61 (d, J=5.2 Hz, 1H), 8.49 (t, J=1.7 Hz, 1H), 7.98 (ddd, J=0.8, 2.3, 8.4 Hz, 1H), 7.52 (t, J=7.9 Hz, 1H), 7.46 (s, 1H), 7.41 (d, J=5.0 Hz, 1H), 7.41 (ddd, J=0.9, 1.4, 7.5 Hz, 1H), 4.34 (q, J=7.1 Hz, 2H), 4.20 (s, 3H), 1.34 (t, J=7.2 Hz, 3H); HRMS (ESI) calcd for C18H17N6O2 [M+H]+349.1408, found 349.1416.

### 5-[2-(3,5-Dimethoxy-phenylamino)-pyrimidin-4-yl]-2-methyl-2H-pyrazole-3-carboxylic acid ethyl ester [(VI), R1=3,5-Dimethoxy-phenyl, R2=methyl, R9=ethyl]

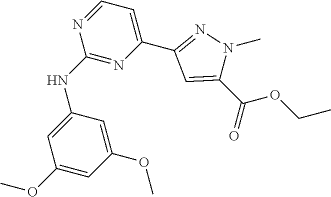 [SMILES: CCOC(=O)c1cc(-c2ccnc(Nc3cc(OC)cc(OC)c3)n2)nn1C]

LC/MS (254 nm) HPLC method (IP) LCO Deca XP-Acetate Buffer Rt 6.84 min

¹H NMR (500 MHz, DMSO-d6) δ ppm 9.67 (s, 1H), 8.54 (d, J=5.0 Hz, 1H), 7.49-7.45 (m, 1H), 7.32 (d, J=5.2 Hz, 1H), 7.20 (d, J=2.3 Hz, 2H), 6.14 (t, J=2.2 Hz, 1H), 4.33 (q, J=7.2 Hz, 2H), 4.19 (s, 3H), 3.76 (s, 6H), 1.34 (t, J=7.1 Hz, 3H); HRMS (ESI) calcd for C19H22N5O4 [M+H]+ 384.1667, found 384.1666.

### 5-[2-(3-Methoxy-phenylamino)-pyrimidin-4-yl]-2-methyl-2H-pyrazole-3-carboxylic acid ethyl ester [(VI), R1=3-Methoxy-phenyl, R2=methyl, R9=ethyl]

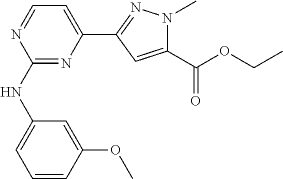 [SMILES: CCOC(=O)c1cc(-c2ccnc(Nc3cccc(OC)c3)n2)nn1C]

LC/MS (254 nm) HPLC method (IP) LCT-Formic Acid Rt 11.81 min

¹H NMR (500 MHz, DMSO-d6) δ ppm 9.70 (s, 1H), 8.54 (d, J=5.0 Hz, 1H), 7.75 (br. s., 1H), 7.46 (s, 1H), 7.32 (d, J=5.0 Hz, 1H), 7.27 (dd, J=0.8, 8.2 Hz, 1H), 7.21-7.15 (m, 1H), 6.57-6.52 (m, 1H), 4.34 (q, J=7.1 Hz, 2H), 4.19 (s, 3H), 3.79 (s, 3H), 1.34 (t, J=7.1 Hz, 3H); HRMS (ESI) calcd for C18H20N5O3 [M+H]+ 354.1561, found 354.1564.

### 2-Methyl-5-[2-(3,4,5-trimethoxy-phenylamino)-pyrimidin-4-yl]-2H-pyrazole-3-carboxylic acid ethyl ester [(VI), R1=3,4,5-trimethoxy-phenyl, R2=methyl, R9=ethyl]

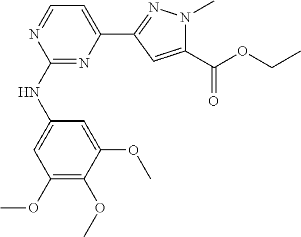 [SMILES: CCOC(=O)c1cc(-c2ccnc(Nc3cc(OC)c(OC)c(OC)c3)n2)nn1C]

LC/MS (254 nm) HPLC method (IP) LCT-Formic Acid Rt 11.04 min

¹H NMR (500 MHz, DMSO-d6) δ ppm 9.59 (s, 1H), 8.53 (d, J=5.0 Hz, 1H), 7.49-7.48 (m, 1H), 7.34 (s, 2H), 7.30 (d, J=5.0 Hz, 1H), 4.33 (q, J=7.1 Hz, 2H), 4.18 (s, 3H), 3.82 (s, 6H), 3.63 (s, 3H), 1.34 (t, J=7.1 Hz, 3H); HRMS (ESI) calcd for C20H24N5O5 [M+H]+ 414.1772, found 414.1772.

### 5-[2-(3-Fluoro-5-methoxy-phenylamino)-pyrimidin-4-yl]-2-methyl-2H-pyrazole-3-carboxylic acid ethyl ester [(VI), R1=3-Fluoro-5-methoxy-phenyl, R2=methyl, R9=ethyl]

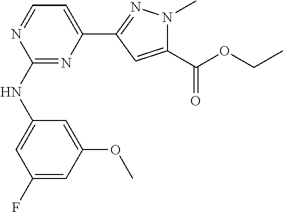 [SMILES: CCOC(=O)c1cc(-c2ccnc(Nc3cc(F)cc(OC)c3)n2)nn1C]

LC/MS (254 nm) HPLC method (IP) LCT-Formic Acid Rt 12.60 min

¹H NMR (500 MHz, DMSO-d6) δ ppm 9.91 (s, 1H), 8.58 (d, J=5.2 Hz, 1H), 7.47-7.42 (m, 2H), 7.37 (d, J=5.0 Hz, 1H), 7.36-7.31 (m, 1H), 6.41 (td, J=2.2, 10.8 Hz, 1H), 4.34 (q, J=7.0 Hz, 2H), 4.20 (s, 3H), 3.79 (s, 3H), 1.34 (t, J=7.1 Hz, 3H); HRMS (ESI) calcd for C18H19FN5O3 [M+H]+ 372.1467, found 372.1465.

### 5-[2-(3-Cyano-5-methoxy-phenylamino)-pyrimidin-4-yl]-2-methyl-2H-pyrazole-3-carboxylic acid ethyl ester [(VI), R1=3-Cyano-5-methoxy-phenyl, R2=methyl, R9=ethyl] 87% yield

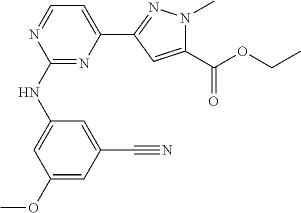 [SMILES: CCOC(=O)c1cc(-c2ccnc(Nc3cc(C#N)cc(OC)c3)n2)nn1C]

LC/MS (254 nm) HPLC method (IP) LCT-Formic Acid Rt 12.08 min

¹H NMR (500 MHz, DMSO-d6) δ ppm 10.09 (s, 1H), 8.61 (d, J=5.2 Hz, 1H), 7.93-7.90 (m, 1H), 7.88 (t, J=1.9 Hz, 1H), 7.46 (s, 1H), 7.41 (d, J=5.2 Hz, 1H), 7.02 (dd, J=1.4, 2.3 Hz, 1H), 4.33 (q, J=7.2 Hz, 2H), 4.20 (s, 3H), 3.84 (s, 3H), 1.34 (t, J=7.1 Hz, 3H); HRMS (ESI) calcd for C19H19N6O3 [M+H]+ 379.1513, found 379.1514.

### 5-[2-(5-Methoxy-pyridin-3-ylamino)-pyrimidin-4-yl]-2-methyl-2H-pyrazole-3-carboxylic acid ethyl ester [(VI), R1=5-Methoxy-pyridin-3-yl, R2=methyl, R9_=ethyl] 37% yield

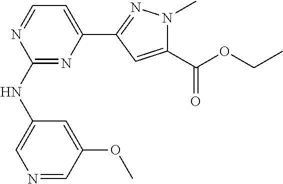 [SMILES: CCOC(=O)c1cc(-c2ccnc(Nc3cncc(OC)c3)n2)nn1C]

LC/MS (254 nm) HPLC method (IP) LCT-Formic Acid Rt 8.62 min

¹H NMR (500 MHz, DMSO-d6) δ ppm 9.97 (s, 1H), 8.59 (d, J=5.0 Hz, 1H), 8.50 (d, J=2.0 Hz, 1H), 8.19 (br. s., 1H), 7.91 (d, J=2.6 Hz, 1H), 7.46 (s, 1H), 7.39 (d, J=5.0 Hz, 1H), 4.34 (q, J=7.1 Hz, 2H), 4.19 (s, 3H), 3.88 (s, 3H), 1.34 (t, J=7.1 Hz, 3H); HRMS (ESI) calcd for C17H19N6O3 [M+H]+ 355.1513, found 355.1518.

### 5-[2-(3,5-Bis-trifluoromethyl-phenylamino)-pyrimidin-4-yl]-2-methyl-2H-pyrazole-3-carboxylic acid ethyl ester [(VI), R1=3,5-Bis-trifluoromethyl-phenyl, R2=methyl, R9=ethyl]

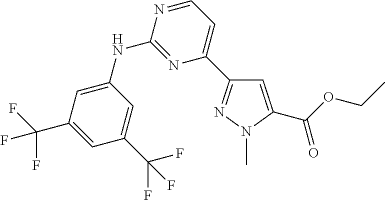 [SMILES: CCOC(=O)c1cc(-c2ccnc(Nc3cc(C(F)(F)F)cc(C(F)(F)F)c3)n2)nn1C]

### 5-[2-(3-Methoxy-5-trifluoromethyl-phenylamino)-pyrimidin-4-yl]-2-methyl-2H-pyrazole-3-carboxylic acid ethyl ester [(VI), R1=3-Methoxy-5-trifluoromethyl-phenyl, R2=methyl, R9=ethyl]

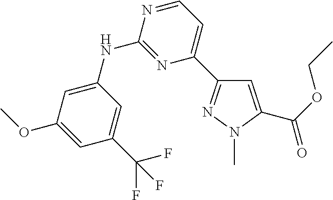 [SMILES: CCOC(=O)c1cc(-c2ccnc(Nc3cc(OC)cc(C(F)(F)F)c3)n2)nn1C]

### 5-[2-(4,6-Dimethyl-pyridin-2-ylamino)-pyrimidin-4-yl]-2-methyl-2H-pyrazole-3-carboxylic acid ethyl ester [(VI), R1=4,6-Dimethyl-pyridin-2-yl, R2=methyl, R9=ethyl]

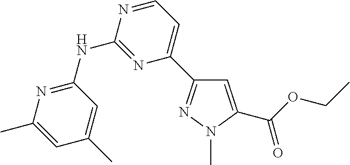 [SMILES: CCOC(=O)c1cc(-c2ccnc(Nc3cc(C)cc(C)n3)n2)nn1C]

### 2-Methyl-5-[2-(3-trifluoromethyl-phenylamino)-pyrimidin-4-yl]-2H-pyrazole-3-carboxylic acid ethyl ester [(VI), R1=3-trifluoromethyl-phenyl, R2=methyl, R9=ethyl]

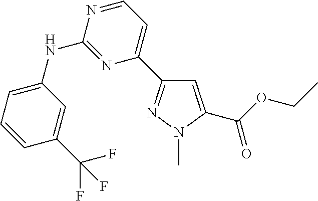 [SMILES: CCOC(=O)c1cc(-c2ccnc(Nc3cccc(C(F)(F)F)c3)n2)nn1C]

### 5-[2-(Benzo[1,3]dioxol-5-ylamino)-pyrimidin-4-yl]-2-methyl-2H-pyrazole-3-carboxylic acid ethyl ester [(VI), R1=Benzo[1,3]dioxol-5-ylamino)-pyrimidin-4-yl, R2=methyl, R9=ethyl]

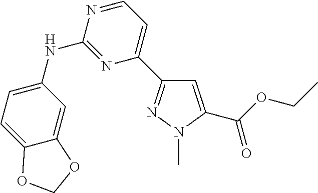 [SMILES: CCOC(=O)c1cc(-c2ccnc(Nc3ccc4c(c3)OCO4)n2)nn1C]

### 5-[2-(3,4-Dimethoxy-phenylamino)-pyrimidin-4-yl]-2-methyl-2H-pyrazole-3-carboxylic acid ethyl ester [(VI), R1=3.4-Dimethoxy-phenylamino)-pyrimidin-4-yl, R2=methyl, R9=ethyl]

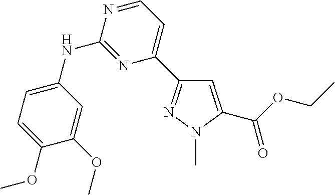 [SMILES: CCOC(=O)c1cc(-c2ccnc(Nc3ccc(OC)c(OC)c3)n2)nn1C]

### Step 5

### Ethyl 3-(2-iodopyrimidin-4-yl)-1-methyl-1H-pyrazole-5-carboxylate [(IX), R2=methyl, R9=ethyl]

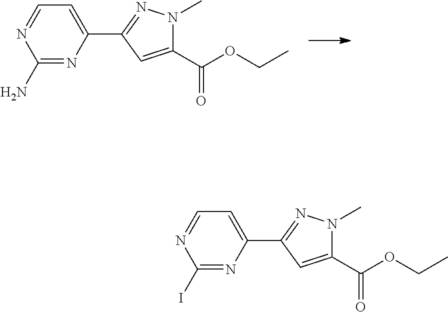 [SMILES: CCOC(=O)c1cc(-c2ccnc(N)n2)nn1C ; CCOC(=O)c1cc(-c2ccnc(I)n2)nn1C]

To a solution of 5-(2-Amino-pyrimidin-4-yl)-2-methyl-2H-pyrazole-3-carboxylic acid ethyl ester (450 mg, 1.82 mmol, 1 eq.) in dioxane dry degassed (11 ml) CuI (156 mg, 0.82 mmol, 0.45 eq.), CsI (708 mg, 2.73 mmol, 1.5 eq.), iodine (323 mg, 1.28 mmol, 0.7 eq.) and iso-amylnitrite (535 ul, 4.01 mmol, 2.2 eq.) were added. The reaction mixture was heated at T=90° C. for two hours, then cooled to room temperature and diluted with water, extracted three times with AcOEt. The organics were collected and washed with ammonia solution 10%, aqueous solution of Na₂S₂O5 5% and brine. The organic layer was dried over anhydrous Na₂SO₄ and evaporated to dryness. The crude was purified by flash chromatography on silica gel (eluant: Hexane/AcOEt 8/2) to afford 230 mg (35% yield) of the title compound.

LC/MS (254 nm) HPLC method (IP) LCQ Deca XP-Acetate Buffer Rt 6.43 min.

¹H NMR (500 MHz, DMSO-d6) δ ppm 1.34 (t, J=7.1 Hz, 3H) 4.19 (s, 3H) 4.34 (q, J=7.1 Hz, 2H) 7.44 (s, 1H) 7.98 (d, J=5.2 Hz, 1H) 8.57 (d, J=5.2 Hz, 1H); HRMS (ESI) calcd for C11H11N4O2 [M+H]+ 359, found 359.0013.

### Step 6

### Ethyl 1-methyl-3-{2-[(1,2,3-trimethyl-1H-indol-5-yl)amino]pyrimidin-4-yl}-1H-pyrazole-5-carboxylate [(VI), R1=1,2,3-trimethyl-1H-indol-5-yl, R2=methyl, R9=ethyl]

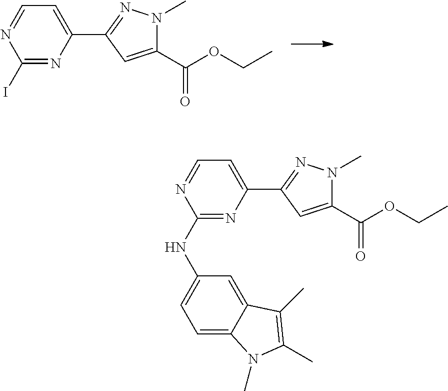 [SMILES: CCOC(=O)c1cc(-c2ccnc(I)n2)nn1C ; CCOC(=O)c1cc(-c2ccnc(Nc3ccc4c(c3)c(C)c(C)n4C)n2)nn1C]

Ethyl 3-(2-iodopyrimidin-4-yl)-1-methyl-1H-pyrazole-5-carboxylate (86 mg, 0.24 mmol, 1 eq.), 1,2,3-trimethyl-1H-indol-5-amine (42 mg, 0.24 mmol, 1 eq.) and K₂CO₃ (99.5 mg, 0.72 mmol, 3 eq.) were suspended in previously degassed dioxane (4 ml) and three cycles vacuum/argon were performed. Xantphos (27.8 mg, 0.048 mmol, 0.2 eq.) and Pd(OAc)₂ (5.4 mg, 0.024 mmol, 0.1 eq.) were added under argon atmosphere and three cycles vacuum/argon were performed. The mixture was heated at 100° C. for 3 hours, then cooled to room temperature and diluted with water, extracted three times with AcOEt, the organics were collected and washed with brine and the volatiles were removed under reduced pressure. The crude solid was purified by flash chromatography on silica gel (eluant: DCM/AcOEt 9/1) to afford 10 mg (10% yield) of the title compound.

LC/MS (254 nm) HPLC method (IP) LCQ Deca XP-Acetate Buffer Rt 7.38 min.

¹H NMR (500 MHz, DMSO-d6) δ ppm 1.35 (t, J=7.1 Hz, 3H) 2.23 (s, 3H) 2.33 (s, 3H) 3.62 (s, 3H) 4.20 (s, 3H) 4.35 (q, J=7.1 Hz, 2H) 7.21 (dd, J=8.5, 1.5 Hz, 1H) 7.22 (d, J=5.0 Hz, 1H) 7.25 (d, J=8.7 Hz, 1H) 7.48 (s, 1H) 8.25 (br. s., 1H) 8.47 (d, J=5.0 Hz, 1H) 9.44 (s, 1H); HRMS (ESI) calcd for C22H24N6O2 [M+H]+ 405.2034, found 405.2033.

### Ethyl 3-{2-[(3-chloro-1-methyl-1H-indol-5-yl)amino]pyrimidin-4-yl}-1-methyl-1H-pyrazole-5-carboxylate [(VI), R1=3-chloro-1-methyl-1H-indol-5-yl, R2=methyl, R9=ethyl]

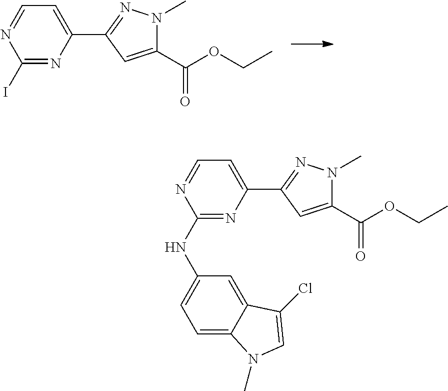 [SMILES: CCOC(=O)c1cc(-c2ccnc(I)n2)nn1C ; CCOC(=O)c1cc(-c2ccnc(Nc3ccc4c(c3)c(Cl)cn4C)n2)nn1C]

Ethyl 3-(2-iodopyrimidin-4-yl)-1-methyl-1H-pyrazole-5-carboxylate (140 mg, 0.39 mmol, 1 eq.), 3-chloro-1-methyl-1H-indol-5-amine (84.5 mg, 0.47 mmol, 1.2 eq.) and caesium carbonate (381 mg, 1.17 mmol, 3 eq.) were suspended in previously degassed dioxane (8 ml) and three cycles vacuum/argon were performed. XPhos (74.5 mg, 0.156 mmol, 0.4 eq.) and Tris(dibenzylideneacetone)dipalladium(0) (71.6 mg, 0.078 mmol, 0.2 eq.) were added under argon atmosphere and three cycles vacuum/argon were performed. The mixture was heated at 100° C. for 7 hours, then cooled to room temperature and diluted with water, extracted three times with AcOEt, the organics were collected and washed with brine and the volatiles were removed under reduced pressure. The crude solid was purified by flash chromatography on silica gel (eluant: DCM/AcOEt 95/5) to afford 24 mg (15% yield) of the title compound.

LC/MS (254 nm) HPLC method (IP) LCQ Deca XP-Acetate Buffer Rt 7.22 min.

¹H NMR (500 MHz, DMSO-d6) δ ppm 1.35 (t, J=7.1 Hz, 3H) 3.76 (s, 3H) 4.21 (s, 3H) 4.34 (q, J=7.2 Hz, 2H) 7.29 (d, J=5.0 Hz, 1H) 7.37 (dd, J=8.9, 1.5 Hz, 1H) 7.43 (d, J=8.8 Hz, 1H) 7.48 (s, 1H) 7.58 (s, 1H) 8.53 (d, J=4.9 Hz, 1H) 8.59 (br. s., 1H) 9.70 (s, 1H); HRMS (ESI) calcd for C20H19ClN6O2 [M+H]+ 411.1331, found 411.1340.

### Step 7

### 2-Methyl-5-(2-m-tolylamino-pyrimidin-4-yl)-2H-pyrazole-3-carboxylic acid [(XI), R1=2-m-tolyl, R2=methyl]100% yield

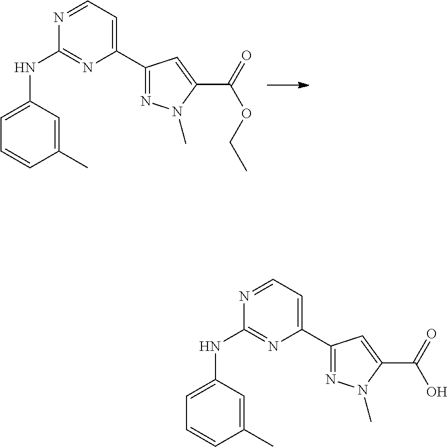 [SMILES: CCOC(=O)c1cc(-c2ccnc(Nc3cccc(C)c3)n2)nn1C ; Cc1cccc(Nc2nccc(-c3cc(C(=O)O)n(C)n3)n2)c1]

Sodium hydroxide 2N (10.5 ml) was added to a solution of 2-Methyl-5-(2-m-tolylamino-pyrimidin-4-yl)-2H-pyrazole-3-carboxylic acid ethyl ester (260 mg, 0.77 mmol, 1 eq.) in ethanol (21 ml) and the mixture was stirred at room temperature for 4 hours. HCl 2N was added to reach pH<5 and the precipitated was filtered, washed with water and Et₂O and dried in vacuum to afford the title compound in quantitative yield.

LC/MS (254 nm) HPLC method (IP) LCQ Deca XP-Acetate Buffer Rt 4.26 min

¹H NMR (500 MHz, DMSO-d6) δ ppm 9.60 (s, 1H), 8.51 (d, J=5.0 Hz, 1H), 7.69 (s, 1H), 7.61 (d, J=7.9 Hz, 1H), 7.38 (s, 1H), 7.29 (d, J=5.2 Hz, 1H), 7.18 (t, J=7.8 Hz, 1H), 6.78 (d, J=7.5 Hz, 1H), 4.18 (s, 3H), 2.30 (s, 3H); HRMS (ESI) calcd for C16H16N5O2 [M+H]+ 310.1299, found 310.1302.

According to this same methodology, but employing suitable substituted derivatives, the following compounds were prepared:

### 5-[2-(3,5-Difluoro-phenylamino)-pyrimidin-4-yl]-2-methyl-2H-pyrazole-3-carboxylic acid [(XI), R1=3,5-Difluoro-phenyl, R2=methyl] quant. yield

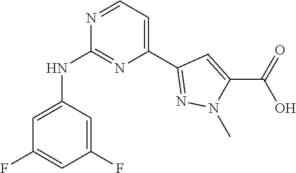 [SMILES: Cn1nc(-c2ccnc(Nc3cc(F)cc(F)c3)n2)cc1C(=O)O]

LC/MS (254 nm) HPLC method (IP) LCQ Deca XP-Acetate Buffer Rt 4.51 min

¹H NMR (500 MHz, DMSO-d6) δ ppm 10.16 (s, 1H), 8.60 (d, J=5.2 Hz, 1H), 7.68-7.58 (m, 2H), 7.42 (d, J=5.2 Hz, 1H), 7.38 (s, 1H), 6.76 (tt, J=2.2, 9.3 Hz, 1H), 4.19 (s, 3H); HRMS (ESI) calcd for C15H12F2N5O2 [M+H]+332.0954, found 332.0956.

### 5-{2-[3-Chloro-4-(4-methyl-piperazin-1-yl)-phenylamino]-pyrimidin-4-yl}-2-methyl-2H-pyrazole-3-carboxylic Acid [(XI), R1=3-Chloro-4-(4-methyl-piperazin-1-yl)-phenyl, R2=methyl] quant. yield

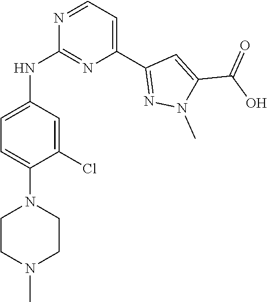 [SMILES: CN1CCN(c2ccc(Nc3nccc(-c4cc(C(=O)O)n(C)n4)n3)cc2Cl)CC1]

LC/MS (254 nm) HPLC method (IP) LCQ Deca XP-Acetate Buffer Rt 3.57 min

¹H NMR (500 MHz, DMSO-d6) δ ppm 11.99 (s, 1H), 9.69 (s, 1H), 8.48 (d, J=5.2 Hz, 1H), 8.03 (d, J=2.4 Hz, 1H), 7.72 (dd, J=2.1, 8.7 Hz, 1H), 7.29 (d, J=5.2 Hz, 1H), 7.19 (s, 1H), 7.15 (d, J=8.8 Hz, 1H), 4.17 (s, 3H), 2.98 (br. s., 4H), 2.35 (br. s., 3H); HRMS (ESI) calcd for C20H23ClN7O2 [M+H]+ 428.1597, found 428.1607.

### 5-[2-(3-Chloro-1-methyl-1H-indol-5-ylamino)-pyrimidin-4-yl]-2-methyl-2H-pyrazole-3-carboxylic acid [(XI), R1=3-Chloro-1-methyl-1H-indol-5-yl, R2=methyl] 93% yield

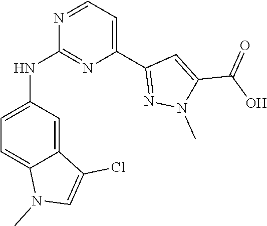 [SMILES: Cn1nc(-c2ccnc(Nc3ccc4c(c3)c(Cl)cn4C)n2)cc1C(=O)O]

LC/MS (254 nm) HPLC method (IP) LCQ Deca XP-Acetate Buffer Rt 4.53 min

¹H NMR (500 MHz, DMSO-d6) δ ppm 13.60 (br. s., 1H), 9.64 (br. s., 1H), 8.50 (br. s., 1H), 8.41 (br. s., 1H), 7.56-7.33 (m, 4H), 7.26 (br. s., 1H), 4.19 (s, 3H), 3.76 (s, 3H); HRMS (ESI) calcd for C18H16ClN6O2 [M+H]+383.1018, found 383.1006.

### 1-methyl-3-{2-[(1,2,3-trimethyl-1H-indol-5-yl)amino]pyrimidin-4-yl}-1H-pyrazole-5-carboxylic acid [(XI), R1=1,2,3-trimethyl-1H-indol-5-yl, R2=methyl]

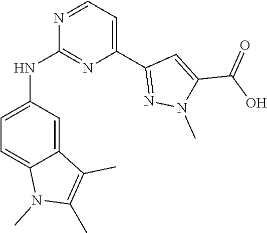 [SMILES: Cc1c(C)n(C)c2ccc(Nc3nccc(-c4cc(C(=O)O)n(C)n4)n3)cc12]

Alternatively the acids or its salt could be prepared as reported below:

### 5-[2-(3,5-Dimethyl-phenylamino)-pyrimidin-4-yl]-2-methyl-2H-pyrazole-3-carboxylic acid [(XI), R1=3,5-Dimethyl-phenyl, R2=methyl]

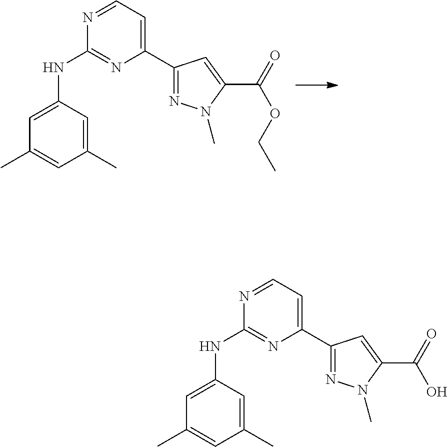

Lithium hydroxide mono hydrate (47.8 mg, 1.138 mmol, 2.5 eq.) dissolved in water (2 ml) and ethanol (1 ml) were sequentially added to a solution of 5-[2-(3,5-Dimethyl-phenylamino)-pyrimidin-4-yl]-2-methyl-2H-pyrazole-3-carboxylic acid ethyl ester (160 mg, 0.455 mmol, 1 eq.) in THE (2 ml). The mixture was stirred at room temperature for 16 h then treated with HCl 1N until pH=3 was reached. The organics were removed under reduced pressure and the precipitate was filtered and rinsed with water and Et₂O to afford 132 mg (90% yield) of the title compound that was used without further purifications.

LC/MS (254 nm) HPLC method (IP) LCO Deca XP-Acetate Buffer Rt 4.55 min

¹H NMR (500 MHz, DMSO-d6) δ ppm 13.66 (br. s, 1H), 9.55 (s, 1H), 8.51 (d, J=5.0 Hz, 1H), 7.48 (br. s, 2H), 7.38 (s, 1H), 7.28 (d, J=5.0 Hz, 1H), 6.62 (br. s, 1H), 4.18 (s, 3H), 2.26 (s, 6H); HRMS (ESI) calcd for C17H18N5O2 [M+H]+ 324.1455, found 324.1454.

According to this same methodology, but employing suitable substituted derivatives, the following compounds were prepared:

### 5-[2-(3-Methoxy-phenylamino)-pyrimidin-4-yl]-2-methyl-2H-pyrazole-3-carboxylic acid [(XI), R1=3-Methoxy-phenyl, R2=methyl] 94% yield

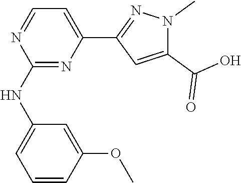 [SMILES: COc1cccc(Nc2nccc(-c3cc(C(=O)O)n(C)n3)n2)c1]

LC/MS (254 nm) HPLC method (IP) LCT-Formic Acid Rt 9.53 min

¹H NMR (500 MHz, DMSO-d6) δ ppm 9.68 (s, 1H), 8.53 (d, J=5.2 Hz, 1H), 7.68 (s, 1H), 7.40 (s, 1H), 7.31 (d, J=5.2 Hz, 2H), 7.21-7.17 (m, 1H), 6.54 (dd, J=2.2, 7.9 Hz, 1H), 4.18 (s, 3H), 3.77 (s, 3H); HRMS (ESI) calcd for C16H16N5O3 [M+H]+ 326.1248, found 326.1248.

### 5-[2-(3,5-Dimethoxy-phenylamino)-pyrimidin-4-yl]-2-methyl-2H-pyrazole-3-carboxylic acid [(XI), R1=3,5-Dimethoxy-phenyl, R2=methyl] 92% yield

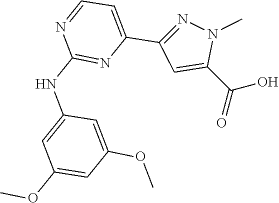 [SMILES: COc1cc(Nc2nccc(-c3cc(C(=O)O)n(C)n3)n2)cc(OC)c1]

LC/MS (254 nm) HPLC method (IP) LCQ Deca XP-Acetate Buffer Rt 4.13 min

¹H NMR (500 MHz, DMSO-d6) δ ppm 9.67 (s, 1H), 8.56 (d, J=5.0 Hz, 1H), 7.43 (s, 1H), 7.35 (d, J=5.0 Hz, 1H), 7.22 (d, J=2.3 Hz, 2H), 6.17 (t, J=2.3 Hz, 1H), 4.21 (s, 3H), 3.81-3.76 (m, 6H); HRMS (ESI) calcd for C17H18N5O4 [M+H]+ 356.1354, found 356.1366.

### 2-Methyl-5-[2-(3,4,5-trimethoxy-phenylamino)-pyrimidin-4-yl]-2H-pyrazole-3-carboxylic acid [(XI), R1=3,4,5-trimethoxy-phenyl, R2=methyl] 80% Yield

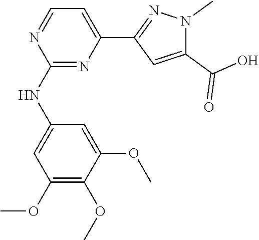 [SMILES: COc1cc(Nc2nccc(-c3cc(C(=O)O)n(C)n3)n2)cc(OC)c1OC]

LC/MS (254 nm) HPLC method (IP) LCT-Formic Acid Rt 9.02 min

¹H NMR (500 MHz, DMSO-d6) δ ppm 9.56 (s, 1H), 8.52 (d, J=5.0 Hz, 1H), 7.44 (s, 1H), 7.33 (s, 2H), 7.29 (d, J=5.2 Hz, 1H), 4.17 (s, 3H), 3.80 (s, 6H), 3.62 (s, 3H); HRMS (ESI) calcd for C18H20N5O5 [M+H]+ 386.1459, found 386.1471.

### 5-[2-(3-Fluoro-5-methoxy-phenylamino)-pyrimidin-4-yl]-2-methyl-2H-pyrazole-3-carboxylic acid [(XI), R1=3-Fluoro-5-methoxy-phenyl, R2=methyl] 99% yield

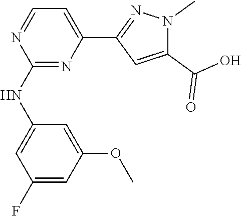 [SMILES: COc1cc(F)cc(Nc2nccc(-c3cc(C(=O)O)n(C)n3)n2)c1]

LC/MS (254 nm) HPLC method (IP) LCT-Formic Acid Rt 10.53 min

¹H NMR (500 MHz, DMSO-d6) δ ppm 13.71 (br. s., 1H), 9.88 (s, 1H), 8.56 (d, J=5.0 Hz, 1H), 7.45-7.30 (m, 4H), 6.41 (td, J=2.2, 10.8 Hz, 1H), 4.18 (s, 3H), 3.78 (s, 3H); HRMS (ESI) calcd for C16H15FN5O3 [M+H]+344.1154, found 344.1140.

### 2-Methyl-5-[2-(1-methyl-1H-pyrazol-4-ylamino)-pyrimidin-4-yl]-2H-pyrazole-3-carboxylic acid [(XI), R1=1-methyl-1H-pyrazol-4-yl, R2=methyl] 72% a yield

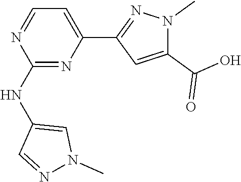 [SMILES: Cn1cc(Nc2nccc(-c3cc(C(=O)O)n(C)n3)n2)cn1]

LC/MS (254 nm) HPLC method (IP) LCT-Formic Acid Rt 5.87 min

¹H NMR (500 MHz, DMSO-d6) δ ppm 13.60 (br. s., 1H), 9.51 (s, 1H), 8.45 (d, J=5.0 Hz, 1H), 7.90 (s, 1H), 7.66-7.53 (m, 1H), 7.37 (br. s., 1H), 7.20 (d, J=5.2 Hz, 1H), 4.18 (s, 3H), 3.82 (s, 3H); HRMS (ESI) calcd for C13H14N7O2 [M+H]+ 300.1204, found 300.1203.

### 2-Methyl-5-[2-(3-trifluoromethyl-phenylamino)-pyrimidin-4-yl]-2H-pyrazole-3-carboxylic acid [(XI), R1=3-trifluoromethyl-phenyl, R2=methyl]

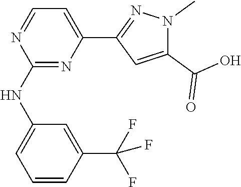 [SMILES: Cn1nc(-c2ccnc(Nc3cccc(C(F)(F)F)c3)n2)cc1C(=O)O]

LC/MS (254 nm) HPLC method (IP) LCT-Formic Acid Rt 11.49 min

¹H NMR (500 MHz, DMSO-d6) δ ppm 9.96 (s, 1H), 8.49 (d, J=5.2 Hz, 1H), 8.44 (s, 1H), 8.04 (d, J=9.0 Hz, 1H), 7.53 (t, J=7.9 Hz, 1H), 7.32 (d, J=5.2 Hz, 1H), 7.26 (d, J=7.6 Hz, 1H), 7.00 (s, 1H), 4.16 (s, 3H); HRMS (ESI) calcd for C16H13F3N5O2 [M+H]+ 364.1016, found 364.1007.

### 5-[2-(3-Cyano-5-methoxy-phenylamino)-pyrimidin-4-yl]-2-methyl-2H-pyrazole-3-carboxylic acid [(XI), R1=3-Cyano-5-methoxy-phenyl, R2=methyl] 96% yield

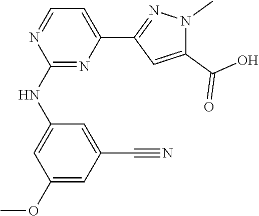 [SMILES: COc1cc(C#N)cc(Nc2nccc(-c3cc(C(=O)O)n(C)n3)n2)c1]

LC/MS (254 nm) HPLC method (IP) LCT-Formic Acid Rt 10.05 min

¹H NMR (500 MHz, DMSO-d6) δ ppm 10.06 (s, 1H), 8.60 (d, J=5.2 Hz, 1H), 7.89 (d, J=1.2 Hz, 2H), 7.41 (t, J=2.5 Hz, 2H), 7.06-6.98 (m, 1H), 4.19 (s, 3H), 3.83 (s, 3H); HRMS (ESI) calcd for C17H15N6O3 [M+H]+351.1200, found 351.1201.

### 5-[2-(5-Methoxy-pyridin-3-ylamino)-pyrimidin-4-yl]-2-methyl-2H-pyrazole-3-carboxylic acid [(XI), R1=5-Methoxy-pyridin-3-yl, R2=methyl] 87% yield

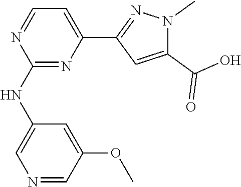 [SMILES: COc1cncc(Nc2nccc(-c3cc(C(=O)O)n(C)n3)n2)c1]

LC/MS (254 nm) HPLC method (IP) LCT-Formic Acid Rt 5.40 min

¹H NMR (500 MHz, DMSO-d6) δ ppm 10.13 (s, 1H), 8.67 (s, 1H), 8.60 (d, J=5.0 Hz, 1H), 8.22 (s, 1H), 8.02 (d, J=2.1 Hz, 1H), 7.46-7.38 (m, 2H), 4.19 (s, 3H), 3.90 (s, 3H); HRMS (ESI) calcd for C15H15N6O3 [M+H]+327.1200, found 327.1199.

### 5-[2-(3,5-Bis-trifluoromethyl-phenylamino)-pyrimidin-4-yl]-2-methyl-2H-pyrazole-3-carboxylic acid [(XI), R1=3,5-Bis-trifluoromethyl-phenyl, R2=methyl]

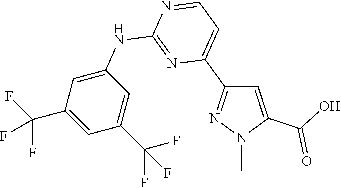 [SMILES: Cn1nc(-c2ccnc(Nc3cc(C(F)(F)F)cc(C(F)(F)F)c3)n2)cc1C(=O)O]

### 5-[2-(3-Methoxy-5-trifluoromethyl-phenylamino)-pyrimidin-4-yl]-2-methyl-2H-pyrazole-3-carboxylic acid [(XI), R1=3-Methoxy-5-trifluoromethyl-phenyl, R2=methyl]

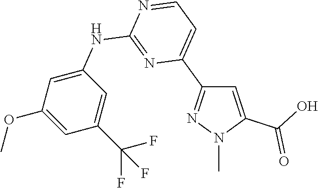 [SMILES: COc1cc(Nc2nccc(-c3cc(C(=O)O)n(C)n3)n2)cc(C(F)(F)F)c1]

### 5-[2-(4,6-Dimethyl-pyridin-2-ylamino)-pyrimidin-4-yl]-2-methyl-2H-pyrazole-3-carboxylic acid [(XI), R1=4,6-Dimethyl-pyridin-2-yl, R2=methyl]

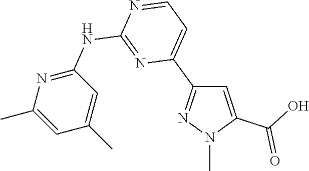 [SMILES: Cc1cc(C)nc(Nc2nccc(-c3cc(C(=O)O)n(C)n3)n2)c1]

### 5-[2-(3,5-Dichloro-phenylamino)-pyrimidin-4-yl]-2-methyl-2H-pyrazole-3-carboxylic acid [(XI), R1=3,5-Dichloro-phenyl, R2=methyl]

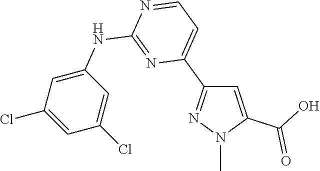 [SMILES: Cn1nc(-c2ccnc(Nc3cc(Cl)cc(Cl)c3)n2)cc1C(=O)O]

### 5-[2-(Benzo[1,3]dioxol-5-ylamino)-pyrimidin-4-yl]-2-methyl-2H-pyrazole-3-carboxylic acid [(XI), R1=2-(Benzo[1,3]dioxol-5-yl, R2=methyl]

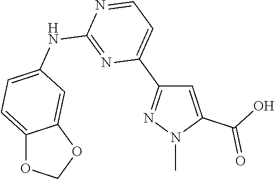 [SMILES: Cn1nc(-c2ccnc(Nc3ccc4c(c3)OCO4)n2)cc1C(=O)O]

### 5-[2-(1,5-Dimethyl-1H-pyrazol-3-ylamino)-pyrimidin-4-yl]-2-methyl-2H-pyrazole-3-carboxylic acid [(XI), R1=1,5-Dimethyl-1H-pyrazol-3-yl, R2=methyl]

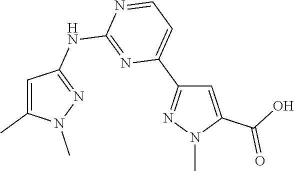 [SMILES: Cc1cc(Nc2nccc(-c3cc(C(=O)O)n(C)n3)n2)nn1C]

### 2-Methyl-5-[2-(tetrahydro-pyran-4-ylamino)-pyrimidin-4-yl]-2H-pyrazole-3-carboxylic acid [(XI), R1=tetrahydro-pyran-4-yl, R2=methyl]

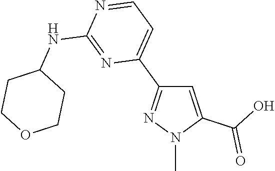 [SMILES: Cn1nc(-c2ccnc(NC3CCOCC3)n2)cc1C(=O)O]

### 5-[2-(3-Cyano-phenylamino)-pyrimidin-4-yl]-2-methyl-2H-pyrazole-3-carboxylic acid [(XI), R1=3-Cyano-phenyl, R2=methyl]

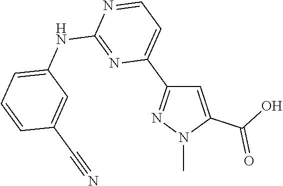 [SMILES: Cn1nc(-c2ccnc(Nc3cccc(C#N)c3)n2)cc1C(=O)O]

### 5-[2-(3,4-Dimethoxy-phenylamino)-pyrimidin-4-yl]-2-methyl-2H-pyrazole-3-carboxylic acid [(XI), R1=3,4-Dimethoxy-phenyl, R2=methyl]

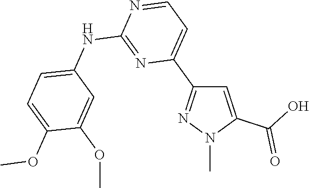 [SMILES: COc1ccc(Nc2nccc(-c3cc(C(=O)O)n(C)n3)n2)cc1OC]

### 5-[2-(3,5-Dimethyl-phenylamino)-pyrimidin-4-yl]-2-ethyl-2H-pyrazole-3-carboxylic acid [(XI), R1=3,5-Dimethyl-phenyl, R2=ethyl]

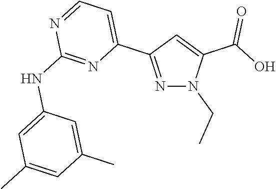 [SMILES: CCn1nc(-c2ccnc(Nc3cc(C)cc(C)c3)n2)cc1C(=O)O]

### 5-[2-(3,5-Dimethyl-phenylamino)-pyrimidin-4-yl]-2H-pyrazole-3-carboxylic acid [(XI), R1=3,5-Dimethyl-phenyl, R2=H]

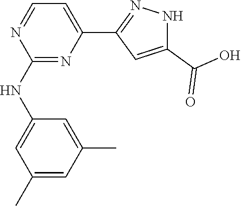 [SMILES: Cc1cc(C)cc(Nc2nccc(-c3cc(C(=O)O)[nH]n3)n2)c1]

### Step 8

### N-[2-(dimethylamino)ethyl]-3-{2-[(3,5-dimethylphenyl)amino]pyrimidin-4-yl}-1-methyl-1H-pyrazole-5-carboxamide (cpd 1) [(I), R1=3,5-dimethylphenyl, R2=methyl, R3=2-(dimethylamino)ethyl, R4=H]

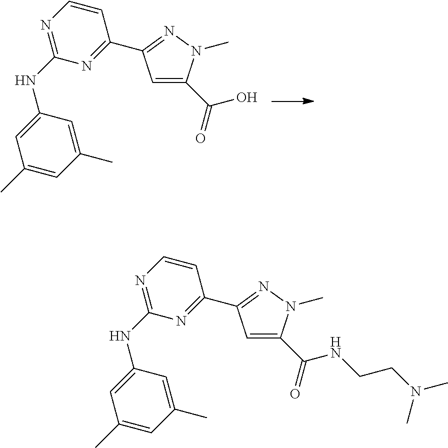

5-[2-(3,5-Dimethyl-phenylamino)-pyrimidin-4-yl]-2-methyl-2H-pyrazole-3-carboxylic acid (25 mg, 0.077 mmol, 1 eq.) and 2-(1H-Benzotriazole-1-yl)-1,1,3,3-tetramethylaminium tetrafluoroborate (TBTU) (37 mg, 0.12 mmol, 1.5 eq) were suspended in DMA dry (1 ml). After 5 minutes of stirring N,N-diisopropylethylamine (DIPEA) (27 ul, 0.15 mmol, 2 eq.) and 2-dimethylamine-ethylamine (12.7 ul, 0.12 mmol, 1.5 eq.) were added and the final mixture was stirred at room temperature for 16 h and then diluted with H₂O (5 ml) extracted three times with AcOEt (3×4 ml). The organic layers were collected and washed with brine dried over Na₂SO₄, filtered and concentrated under reduced pressure. The crude compound was purified by flash chromatography on silica gel (eluent DCM/MeOH 9:1) to provide the title compound in 52% yield.

LC/MS (254 nm) HPLC method (IP) LCQ Deca XP-Acetate Buffer Rt 5.23 min

¹H NMR (500 MHz, DMSO-d6) δ ppm 9.47 (s, 1H), 8.71 (t, J=5.1 Hz, 1H), 8.49 (d, J=5.2 Hz, 1H), 7.47 (s, 2H), 7.42 (s, 1H), 7.25 (d, J=5.0 Hz, 1H), 6.61 (s, 1H), 4.14 (s, 3H), 3.36 (q, J=6.0 Hz, 2H), 2.47 (br. s, 2H), 2.27 (s, 6H), 2.24 (br. s., 6H); HRMS (ESI) calcd for C21H27N7O [M+H]+ 394.235, found 394.2357.

According to this same methodology, but employing suitable amine derivatives eventually both salified or protected, the following compounds were prepared:

### 3-{2-[(3,5-dimethylphenyl)amino]pyrimidin-4-yl}-N-[(2R)-1-hydroxypropan-2-yl]-1-methyl-1H-pyrazole-5-carboxamide (cpd 2) [(I), R1=3,5-dimethylphenyl, R2=methyl, R3=(2R)-1-hydroxypropan-2-yl, R4=H]

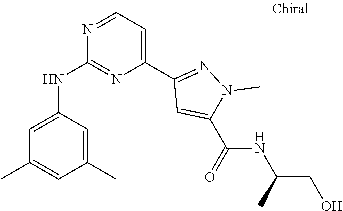 [SMILES: Cc1cc(C)cc(Nc2nccc(-c3cc(C(=O)N[C@H](C)CO)n(C)n3)n2)c1]

¹H NMR (500 MHz, DMSO-d6) δ ppm 9.46 (s, 1H), 8.49 (d, J=5.0 Hz, 1H), 8.45 (d, J=8.2 Hz, 1H), 7.47 (s, 3H), 7.25 (d, J=5.0 Hz, 1H), 6.61 (s, 1H), 4.75 (t, J=5.8 Hz, 1H), 4.13 (s, 3H), 3.98 (qd, J=6.5, 7.7 Hz, 1H), 3.47-3.41 (m, 1H), 3.34-3.30 (m, 1H), 2.27 (s, 6H), 1.12 (d, J=6.7 Hz, 3H); HRMS (ESI) calcd for C20H24N6O2 [M+H]+ 381.2034, found 381.2031.

### 3-{2-[(3,5-dimethylphenyl)amino]pyrimidin-4-yl}-N-[(2S)-1-hydroxypropan-2-yl]-1-methyl-1H-pyrazole-5-carboxamide (cpd 3) [(I), R1=3,5-dimethylphenyl, R2=methyl, R3=(2S)-1-hydroxypropan-2-yl, R4=H]

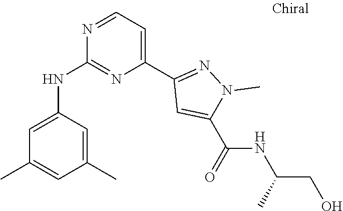 [SMILES: Cc1cc(C)cc(Nc2nccc(-c3cc(C(=O)N[C@@H](C)CO)n(C)n3)n2)c1]

¹H NMR (500 MHz, DMSO-d6) δ ppm 9.46 (s, 1H), 8.49 (d, J=5.0 Hz, 1H), 8.45 (d, J=8.1 Hz, 1H), 7.47 (s, 2H), 7.25 (d, J=5.2 Hz, 1H), 6.61 (s, 1H), 4.74 (t, J=5.8 Hz, 1H), 4.13 (s, 3H), 4.03-3.91 (m, 1H), 3.44 (td, J=5.6, 10.9 Hz, 1H), 2.27 (s, 6H), 1.12 (d, J=6.7 Hz, 3H); HRMS (ESI) calcd for C20H24N6O2 [M+H]+ 381.2034, found 381.2036.

### 3-{2-[(3,5-dimethylphenyl)amino]pyrimidin-4-yl}-1-methyl-N-(propan-2-yl)-1H-pyrazole-5-carboxamide (cpd 4) [(I), R1=3,5-dimethylphenyl, R2=methyl, R3=propan-2-yl, R4=H]

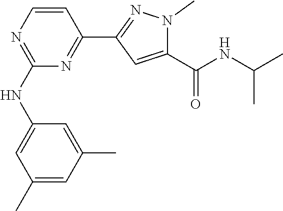 [SMILES: Cc1cc(C)cc(Nc2nccc(-c3cc(C(=O)NC(C)C)n(C)n3)n2)c1]

¹H NMR (500 MHz, DMSO-d6) δ ppm 9.46 (s, 1H), 8.59 (d, J=7.9 Hz, 1H), 8.49 (d, J=5.0 Hz, 1H), 7.48 (s, 2H), 7.46 (s, 1H), 7.25 (d, J=5.0 Hz, 1H), 6.61 (s, 1H), 4.13 (s, 3H), 4.11-4.01 (m, 1H), 2.27 (s, 6H), 1.16 (d, J=6.6 Hz, 6H); HRMS (ESI) calcd for C20H24N6O [M+H]+ 365.2085, found 365.2087.

### N-[2-(dimethylamino)ethyl]-3-{2-[(3,5-dimethylphenyl)amino]pyrimidin-4-yl}-N,1-dimethyl-1H-pyrazole-5-carboxamide (cpd 5) [(I), R1=3,5-dimethylphenyl, R2=methyl, R3=[2-(dimethylamino)ethyl, R4=methyl]

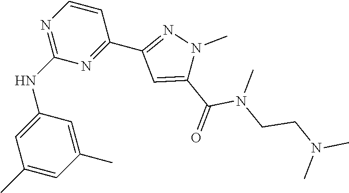

¹H NMR (500 MHz, DMSO-d6) δ ppm 9.51 (d, J=5.6 Hz, 1H), 8.49 (br. s., 1H), 7.47 (s, 2H), 7.26 (br. s., 1H), 7.09-6.91 (m, 1H), 6.60 (s, 1H), 4.05-3.83 (m, 3H), 3.75-3.43 (m, 2H), 3.18-2.95 (m, 3H), 2.34 (br. s., 3H), 2.25 (s, 6H), 1.95 (br. s., 3H); HRMS (ESI) calcd for C22H29N7O [M+H]+ 408.2507, found 408.2518.

### 3-{2-[(3,5-dimethylphenyl)amino]pyrimidin-4-yl}-1-methyl-N-[2-(methylamino)ethyl]-1H-pyrazole-5-carboxamide hydrochloride (cpd 6) [(I), R1=3,5-dimethylphenyl, R2=methyl, R3=2-(methylamino)ethyl, R4=H]

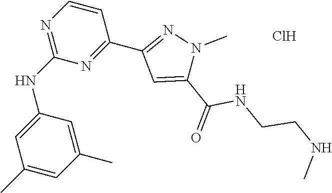 [SMILES: CNCCNC(=O)c1cc(-c2ccnc(Nc3cc(C)cc(C)c3)n2)nn1C ; Cl]

¹H NMR (500 MHz, DMSO-d6) δ ppm 9.49 (s, 1H), 9.00 (t, J=5.6 Hz, 1H), 8.67 (br. s., 2H), 8.50 (d, J=5.0 Hz, 1H), 7.46 (s, 2H), 7.26 (d, J=5.2 Hz, 1H), 6.62 (s, 1H), 4.16 (s, 3H), 3.58-3.53 (m, 2H), 3.09 (quin, J=5.9 Hz, 2H), 2.59 (t, J=5.4 Hz, 3H), 2.31-2.22 (m, 6H); HRMS (ESI) calcd for C20H26N7O Cl [M+H]+ 380.2194, found 380.2186.

### N-(2-aminoethyl)-3-{2-[(3,5-dimethylphenyl)amino]pyrimidin-4-yl}-1-methyl-1H-pyrazole-5-carboxamide hydrochloride (cpd 7) [(I), R1=3,5-dimethylphenyl, R2=methyl, R3=2-aminoethyl, R4=H]

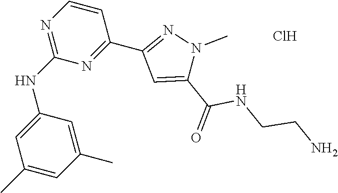 [SMILES: Cc1cc(C)cc(Nc2nccc(-c3cc(C(=O)NCCN)n(C)n3)n2)c1 ; Cl]

¹H NMR (500 MHz, DMSO-d6) δ ppm 9.49 (s, 1H), 8.94 (t, J=5.7 Hz, 1H), 8.50 (d, J=5.2 Hz, 1H), 7.92 (br. s., 3H), 7.48 (s, 1H), 7.46 (s, 2H), 7.26 (d, J=5.2 Hz, 1H), 6.62 (s, 1H), 4.16 (s, 3H), 3.50 (quin, J=5.8 Hz, 2H), 2.98 (sxt, J=5.9 Hz, 2H), 2.27 (s, 6H); HRMS (ESI) calcd for C19H24N7O Cl [M+H]+ 366.2037, found 366.2033.

### N-(azetidin-3-yl)-3-{2-[(3,5-dimethylphenyl)amino]pyrimidin-4-yl}-1-methyl-1H-pyrazole-5-carboxamide trifluoroacetate (cpd 8) [(I), R1=3,5-dimethylphenyl, R2=methyl, R3=azetidin-3-yl, R4=H]

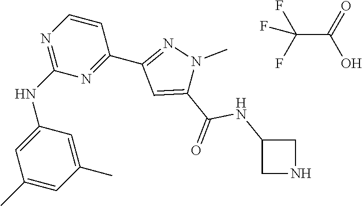

¹H NMR (DMSO-d6, 500 MHz): δ ppm 9.47 (s, 1H), 9.41 (d, J=7.0 Hz, 1H), 8.68 (br. s., 2H), 8.51 (d, J=5.0 Hz, 1H), 7.50 (s, 1H), 7.46 (s, 2H), 7.27 (d, J=5.0 Hz, 1H), 6.63 (s, 1H), 4.82 (sxt, J=7.8 Hz, 1H), 4.16-4.24 (m, 2H), 4.15 (s, 3H), 4.01-4.12 (m, 2H), 2.28 ppm (s, 6H); HRMS (ESI) calcd for C22H24N7O3F3 [M+H]+ 378.2037, found 378.2034.

### 3-{2-[(3,5-dimethylphenyl)amino]pyrimidin-4-yl}-1-methyl-N-[2-(morpholin-4-yl)ethyl]-1H-pyrazole-5-carboxamide (cpd 9) [(I), R1=3,5-dimethylphenyl, R2=methyl, R3=2-morpholin-4-ylethyl), R4=H]

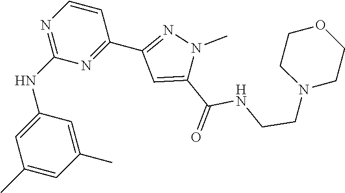 [SMILES: Cc1cc(C)cc(Nc2nccc(-c3cc(C(=O)NCCN4CCOCC4)n(C)n3)n2)c1]

¹H NMR (500 MHz, DMSO-d6) δ ppm 9.46 (s, 1H), 8.72 (t, J=5.6 Hz, 1H), 8.49 (d, J=5.0 Hz, 1H), 7.46 (s, 2H), 7.41 (s, 1H), 7.26 (d, J=5.0 Hz, 1H), 6.62 (s, 1H), 4.14 (s, 3H), 3.57 (t, J=4.6 Hz, 4H), 3.37 (q, J=6.3 Hz, 2H), 2.46 (t, J=6.7 Hz, 2H), 2.41 (br. s., 4H), 2.27 (s, 6H); HRMS (ESI) calcd for C23H29N7O2 [M+H]+ 436.2456, found 436.2447.

### N-[2-(diethylamino)ethyl]-3-{2-[(3,5-dimethylphenyl)amino]pyrimidin-4-yl}-1-methyl-1H-pyrazole-5-carboxamide (cpd 10) [(1), R1=3,5-dimethylphenyl), R2=methyl, R3=2-(diethylamino)ethyl, R4=H]

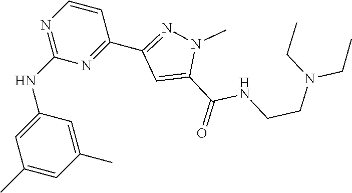 [SMILES: CCN(CC)CCNC(=O)c1cc(-c2ccnc(Nc3cc(C)cc(C)c3)n2)nn1C]

¹H NMR (500 MHz, DMSO-d6) δ ppm 9.46 (s, 1H), 8.90 (br. t, J=4.5 Hz, 1H), 8.50 (d, J=5.0 Hz, 1H), 7.46 (s, 2H), 7.43 (s, 1H), 7.26 (d, J=5.0 Hz, 1H), 6.62 (s, 1H), 4.15 (s, 3H), 3.51 (br. q, J=5.3 Hz, 2H), 3.03 (br. s., 6H), 2.27 (s, 6H), 1.20-1.03 (m, 6H); HRMS (ESI) calcd for C23H31N7O [M+H]+ 422.2663, found 422.2666.

### N-[(1R,2R)-2-aminocyclohexyl]-3-{2-[(3,5-dimethylphenyl)amino]pyrimidin-4-yl}-1-methyl-1H-pyrazole-5-carboxamide hydrochloride (cpd 11) [(I), R1=3,5-dimethylphenyl), R2=methyl, R3=(1R,2R)-2-aminocyclohexyl, R4=H]

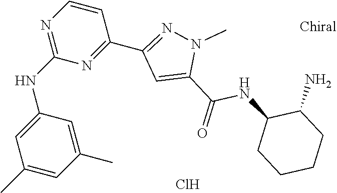 [SMILES: Cc1cc(C)cc(Nc2nccc(-c3cc(C(=O)N[C@@H]4CCCC[C@H]4N)n(C)n3)n2)c1 ; Cl]

¹H NMR (500 MHz, DMSO-d6) δ ppm 9.46 (s, 1H), 8.80 (d, J=8.8 Hz, 1H), 8.50 (d, J=5.0 Hz, 1H), 8.06 (br. s., 3H), 7.51 (s, 1H), 7.47 (s, 2H), 7.26 (d, J=5.0 Hz, 1H), 6.62 (s, 1H), 4.15 (s, 3H), 3.92-3.79 (m, 1H), 3.03-2.94 (m, 1H), 2.27 (s, 6H), 2.07 (d, J=13.4 Hz, 1H), 1.86 (d, J=13.6 Hz, 1H), 1.73 (d, J=8.1 Hz, 2H), 1.52-1.35 (m, 2H), 1.33-1.18 (m, J=11.2, 11.2 Hz, 2H); HRMS (ESI) calcd for C23H30N7O Cl [M+H]+ 420.2507, found 420.2503.

### 3-{2-[(3,5-dimethylphenyl)amino]pyrimidin-4-yl}-1-methyl-N-[2-(propan-2-ylamino)ethyl]-1H-pyrazole-5-carboxamide hydrochloride (cpd 12) [(I), R1=3,5-dimethylphenyl, R2=methyl, R3=2-(propan-2-ylamino)ethyl, R4=H]

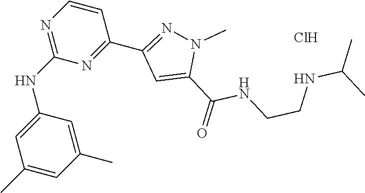 [SMILES: Cc1cc(C)cc(Nc2nccc(-c3cc(C(=O)NCCNC(C)C)n(C)n3)n2)c1 ; Cl]

¹H NMR (500 MHz, DMSO-d6) δ ppm 9.46 (s, 1H), 8.98 (t, J=5.6 Hz, 1H), 8.50 (d, J=5.0 Hz, 1H), 8.51 (br. s., 2H), 7.48 (s, 1H), 7.46 (s, 2H), 7.26 (d, J=5.0 Hz, 1H), 6.62 (s, 1H), 4.16 (s, 3H), 3.56 (q, J=5.9 Hz, 2H), 3.08 (t, J=5.6 Hz, 2H), 2.27 (s, 6H), 1.23 (d, J=6.4 Hz, 6H); HRMS (ESI) calcd for C22H30N7O Cl [M+H]+ 408.2507, found 408.2502.

### 3-{2-[(3,5-dimethylphenyl)amino]pyrimidin-4-yl}-1-methyl-1H-pyrazole-5-carboxamide (cpd 13) [(I), R1=3,5-dimethylphenyl, R2=methyl, R3=H, R4=H]

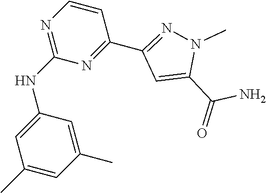 [SMILES: Cc1cc(C)cc(Nc2nccc(-c3cc(C(N)=O)n(C)n3)n2)c1]

¹H NMR (500 MHz, DMSO-d6) δ ppm 9.46 (s, 1H), 8.49 (d, J=5.0 Hz, 1H), 8.21 (br. s., 1H), 7.62 (br. s., 1H), 7.47 (s, 2H), 7.44 (s, 1H), 7.25 (d, J=5.0 Hz, 1H), 6.61 (s, 1H), 4.14 (s, 3H), 2.27 (s, 6H); HRMS (ESI) calcd for C17H18N6O [M+H]+ 323.1615, found 323.1611.

### 3-{2-[(3,5-dimethylphenyl)amino]pyrimidin-4-yl}-N,1-dimethyl-1H-pyrazole-5-carboxamide (cpd 14) [(I), R1=(3,5-dimethylphenyl), R2=methyl, R3=methyl, R4=H]

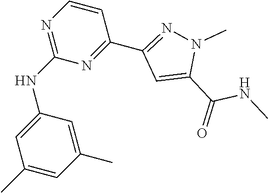

¹H NMR (500 MHz, DMSO-d6) δ ppm 9.45 (s, 1H), 8.73 (d, J=4.7 Hz, 1H), 8.49 (d, J=5.0 Hz, 1H), 7.46 (s, 2H), 7.39 (s, 1H), 7.25 (d, J=5.0 Hz, 1H), 6.61 (s, 1H), 4.14 (s, 3H), 2.77 (d, J=4.6 Hz, 3H), 2.27 (s, 6H); HRMS (ESI) calcd for C18H20N6O [M+H]+ 337.1772, found 337.1775.

### 3-{2-[(3,5-dimethylphenyl)amino]pyrimidin-4-yl}-N,N,1-trimethyl-1H-pyrazole-5-carboxamide (cpd 15) [(I), R1=3,5-dimethylphenyl, R2=methyl, R3=dimethylamino, R4=methyl]

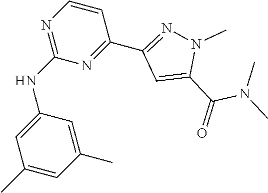

¹H NMR (500 MHz, DMSO-d6) δ ppm 9.51 (s, 1H), 8.50 (d, J=5.2 Hz, 1H), 7.47 (s, 2H), 7.27 (d, J=5.0 Hz, 1H), 7.04 (s, 1H), 6.60 (s, 1H), 3.96 (s, 3H), 3.11 (s, 3H), 3.04 (s, 3H), 2.25 (s, 6H); HRMS (ESI) calcd for C19H22N6O [M+H]+ 351.1928, found 351.1925.

### 3-{2-[(3,5-dimethylphenyl)amino]pyrimidin-4-yl}-N-(2-methoxyethyl)-1-methyl-1H-pyrazole-5-carboxamide (cpd 16) [(I), R1=3,5-dimethylphenyl, R2=methyl, R3=2-methoxyethyl, R4=H]

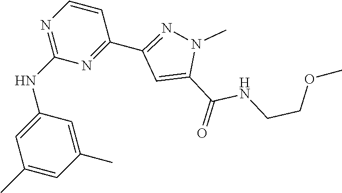

¹H NMR (500 MHz, DMSO-d6) δ ppm 9.47 (s, 1H), 8.82 (t, J=5.5 Hz, 1H), 8.49 (d, J=5.2 Hz, 1H), 7.47 (s, 2H), 7.45 (s, 1H), 7.25 (d, J=5.0 Hz, 1H), 6.61 (s, 1H), 4.14 (s, 3H), 3.49-3.44 (m, 2H), 3.43-3.38 (m, 2H), 3.27 (s, 3H), 2.27 (s, 6H); HRMS (ESI) calcd for C20H24N6O2 [M+H]+ 381.2034, found 381.2034.

### 3-{2-[(3,5-dimethylphenyl)amino]pyrimidin-4-yl}-N-(2-fluoroethyl)-1-methyl-1H-pyrazole-5-carboxamide (cpd 17) [(I), R1=3,5-dimethylphenyl, R2=methyl, R3=2-fluoroethyl, R4=H]

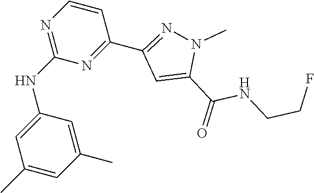 [SMILES: Cc1cc(C)cc(Nc2nccc(-c3cc(C(=O)NCCF)n(C)n3)n2)c1]

¹H NMR (500 MHz, DMSO-d6) δ ppm 9.46 (s, 1H), 9.01 (t, J=5.5 Hz, 1H), 8.50 (d, J=5.0 Hz, 1H), 7.49 (s, 1H), 7.46 (s, 2H), 7.25 (d, J=5.2 Hz, 1H), 6.61 (s, 1H), 4.62-4.47 (m, 2H), 4.15 (s, 3H), 3.62-3.50 (m, 2H), 2.27 (s, 6H); HRMS (ESI) calcd for C19H21N6O F [M+H]+ 369.1834, found 369.1831.

### 3-(2-{[3,5-bis(trifluoromethyl)phenyl]amino}pyrimidin-4-yl)-N-[2-(dimethylamino)ethyl]-1-methyl-1H-pyrazole-5-carboxamide hydrochloride (cpd 18) [(I), R1=3,5-bis(trifluoromethyl)phenyl, R2=methyl, R3=2-(dimethylamino)ethyl, R4=H]

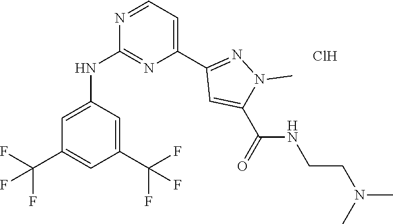 [SMILES: CN(C)CCNC(=O)c1cc(-c2ccnc(Nc3cc(C(F)(F)F)cc(C(F)(F)F)c3)n2)nn1C ; Cl]

¹H NMR (500 MHz, DMSO-d6) δ ppm 10.41 (s, 1H), 9.72 (br. s., 1H), 8.98 (t, J=5.5 Hz, 1H), 8.66 (d, J=5.2 Hz, 1H), 8.60 (s, 2H), 7.62 (s, 1H), 7.45 (s, 1H), 7.42 (d, J=5.0 Hz, 1H), 4.17 (s, 3H), 3.61 (q, J=5.8 Hz, 2H), 3.26 (t, J=5.0 Hz, 1H), 2.84 (s, 6H); HRMS (ESI) calcd for C21H22N7O F6Cl [M+H]+ 502.1785, found 502.1777.

### 3-(2-{[3,5-bis(trifluoromethyl)phenyl]amino}pyrimidin-4-yl)-N-[(2S)-1-hydroxypropan-2-yl]-1-methyl-1H-pyrazole-5-carboxamide (cpd 19) [(I), R1=3,5-bis(trifluoromethyl)phenyl, R2=methyl, R3=(2S)-1-hydroxypropan-2-yl, R4=H]

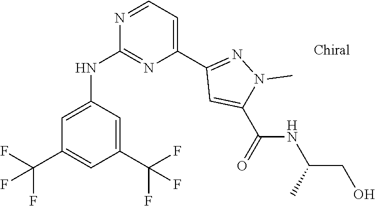

¹H NMR (500 MHz, DMSO-d6) δ ppm 10.41 (s, 1H), 8.64 (d, J=5.2 Hz, 1H), 8.61 (s, 2H), 8.37 (d, J=8.1 Hz, 1H), 7.61 (s, 1H), 7.44 (s, 1H), 7.41 (d, J=5.2 Hz, 1H), 4.75 (t, J=5.8 Hz, 1H), 4.13 (s, 3H), 4.03-3.92 (m, 1H), 3.45 (td, J=5.6, 10.9 Hz, 1H), 3.34-3.30 (m, 1H), 1.13 (d, J=6.7 Hz, 3H); HRMS (ESI) calcd for C20H18N6O2F6 [M+H]+ 489.1468, found 489.146.

### 3-{2-[(3,5-dimethylphenyl)amino]pyrimidin-4-yl}-1-methyl-N-(1H-pyrazol-3-yl)-1H-pyrazole-5-carboxamide (cpd 20) [(I), R1=3,5-dimethylphenyl, R2=methyl, R3=1H-pyrazol-3-yl, R4=H]

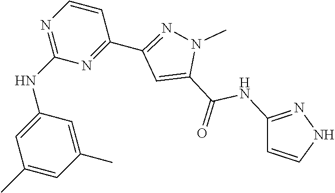 [SMILES: Cc1cc(C)cc(Nc2nccc(-c3cc(C(=O)Nc4cc[nH]n4)n(C)n3)n2)c1]

¹H NMR (500 MHz, DMSO-d6) δ ppm 12.50 (br. s., 1H), 11.16 (s, 1H), 9.49 (s, 1H), 8.51 (d, J=5.2 Hz, 1H), 7.72 (s, 1H), 7.68 (d, J=2.1 Hz, 1H), 7.48 (s, 2H), 7.26 (d, J=5.0 Hz, 1H), 6.61 (br. s, 2H), 4.19 (s, 3H), 2.27 (s, 6H); HRMS (ESI) calcd for C20H20N8O [M+H]+ 389.1833, found 389.1833.

### (3-{2-[(3,5-dimethylphenyl)amino]pyrimidin-4-yl}-1-methyl-1H-pyrazol-5-yl)(4-methylpiperazin-1-yl)methanone (cpd 21) [(I), R1=(3,5-dimethylphenyl), R2=methyl, NR3R4=4-methylpiperazin-1-yl]

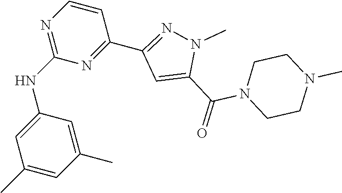

¹H NMR (500 MHz, DMSO-d6) δ ppm 9.52 (s, 1H), 8.50 (d, J=5.0 Hz, 1H), 7.47 (s, 2H), 7.27 (d, J=5.0 Hz, 1H), 6.97 (s, 1H), 6.61 (s, 1H), 3.95 (s, 3H), 3.79-3.45 (m, 4H), 2.37 (br. s., 4H), 2.26 (s, 6H), 2.21 (s, 3H); HRMS (ESI) calcd for C22H27N7O [M+H]+ 406.235, found 406.2351.

### N-[2-(acetylamino)ethyl]-3-{2-[(3,5-dimethylphenyl)amino]pyrimidin-4-yl}-1-methyl-1H-pyrazole-5-carboxamide (cpd 22) [(I), R1=3,5-dimethylphenyl, R2=methyl, R3=2-acetamidoethyl, R4=H]

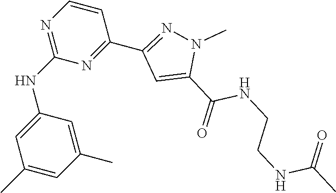 [SMILES: CC(=O)NCCNC(=O)c1cc(-c2ccnc(Nc3cc(C)cc(C)c3)n2)nn1C]

¹H NMR (500 MHz, DMSO-d6) δ ppm 9.46 (s, 1H), 8.79 (t, J=5.6 Hz, 1H), 8.49 (d, J=5.0 Hz, 1H), 7.98 (t, J=5.7 Hz, 1H), 7.46 (s, 2H), 7.42 (s, 1H), 7.25 (d, J=5.0 Hz, 1H), 6.61 (s, 1H), 4.14 (s, 3H), 3.31-3.25 (m, 2H), 3.24-3.16 (m, 2H), 2.27 (s, 6H), 1.81 (s, 3H); HRMS (ESI) calcd for C21H25N7O2 [M+H]+ 408.2143, found 408.2156.

### N-(2-amino-2-oxoethyl)-3-{2-[(3,5-dimethylphenyl)amino]pyrimidin-4-yl}-1-methyl-1H-pyrazole-5-carboxamide (cpd 23) [(I), R1=3,5-dimethylphenyl, R2=methyl, R3=(2-amino-2-oxoethyl), R4=H]

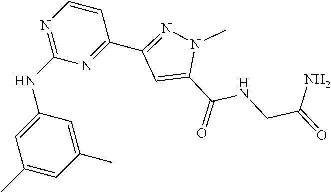 [SMILES: Cc1cc(C)cc(Nc2nccc(-c3cc(C(=O)NCC(N)=O)n(C)n3)n2)c1]

¹H NMR (500 MHz, DMSO-d6) δ ppm 9.47 (s, 1H), 8.94 (t, J=6.0 Hz, 1H), 8.50 (d, J=5.0 Hz, 1H), 7.48 (s, 1H), 7.46 (s, 2H), 7.44 (br. s, 1H), 7.26 (d, J=5.2 Hz, 1H), 7.09 (br. s, 1H), 6.61 (s, 1H), 4.14 (s, 3H), 3.80 (d, J=6.1 Hz, 2H), 2.27 (s, 6H); HRMS (ESI) calcd for C19H21N7O2 [M+H]+ 380.183, found 380.1829.

### 3-{2-[(3,5-dimethylphenyl)amino]pyrimidin-4-yl}-1-methyl-N-(2,2,2-trifluoroethyl)-1H-pyrazole-5-carboxamide (cpd 24) [(I), R1=3,5-dimethylphenyl, R2=methyl, R3=2,2,2-trifluoroethylamino, R4=H]

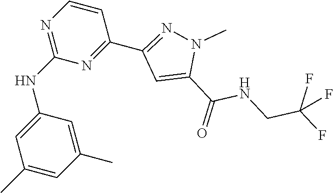 [SMILES: Cc1cc(C)cc(Nc2nccc(-c3cc(C(=O)NCC(F)(F)F)n(C)n3)n2)c1]

¹H NMR (500 MHz, DMSO-d6) δ ppm 9.49 (s, 1H), 9.43 (t, J=6.3 Hz, 1H), 8.51 (d, J=5.0 Hz, 1H), 7.56 (s, 1H), 7.47 (s, 2H), 7.26 (d, J=5.0 Hz, 1H), 6.62 (s, 1H), 4.15 (s, 3H), 4.09 (dq, J=6.1, 9.6 Hz, 2H), 2.27 (s, 6H); HRMS (ESI) calcd for C19H19N6O F3 [M+H]+ 405.1645, found 405.1648.

### 3-{2-[(3,5-dimethylphenyl)amino]pyrimidin-4-yl}-1-methyl-N-[(1-methyl-1H-imidazol-5-yl)methyl]-1H-pyrazole-5-carboxamide (cpd 25) [(I), R1=3,5-dimethylphenyl, R2=methyl, R3=(3-methylimidazol-4-yl)methyl, R4=H]

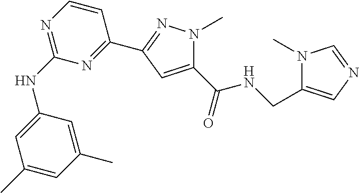 [SMILES: Cc1cc(C)cc(Nc2nccc(-c3cc(C(=O)NCc4cncn4C)n(C)n3)n2)c1]

¹H NMR (500 MHz, DMSO-d6) δ ppm 9.45 (s, 1H), 9.16 (t, J=5.3 Hz, 1H), 8.49 (d, J=5.0 Hz, 1H), 7.56 (s, 1H), 7.48 (s, 1H), 7.46 (s, 2H), 7.25 (d, J=5.2 Hz, 1H), 6.84 (s, 1H), 6.61 (s, 1H), 4.44 (d, J=5.3 Hz, 2H), 4.15 (s, 3H), 3.63 (s, 3H), 2.25 (s, 6H); HRMS (ESI) calcd for C22H24N8O [M+H]+ 417.2146, found 417.2155.

### 3-{2-[(3,5-dimethylphenyl)amino]pyrimidin-4-yl}-N-[(2S)-1-hydroxy-3-methylbutan-2-yl]-1-methyl-1H-pyrazole-5-carboxamide (cpd 26) [(I), R1=3,5-dimethylphenyl, R2=methyl, R3=(2S)-1-hydroxy-3-methylbutan-2-yl, R4=H]

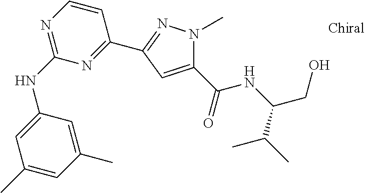 [SMILES: Cc1cc(C)cc(Nc2nccc(-c3cc(C(=O)N[C@H](CO)C(C)C)n(C)n3)n2)c1]

¹H NMR (500 MHz, DMSO-d6) δ ppm 9.48 (s, 1H), 8.49 (d, J=5.0 Hz, 1H), 8.36 (d, J=9.0 Hz, 1H), 7.51 (s, 1H), 7.49 (s, 2H), 7.26 (d, J=5.0 Hz, 1H), 6.61 (s, 1H), 4.59 (t, J=5.6 Hz, 1H), 4.12 (s, 3H), 3.86-3.74 (m, 1H), 3.57-3.50 (m, 1H), 3.50-3.44 (m, 1H), 2.26 (s, 6H), 1.97-1.85 (m, J=6.7, 6.7, 6.7, 6.7, 6.7, 6.7, 6.7 Hz, 1H), 0.91 (d, J=6.9 Hz, 3H), 0.89 (d, J=6.9 Hz, 3H); HRMS (ESI) calcd for C22H28N6O2 [M+H]+ 409.2347, found 409.2352.

### 3-{2-[(3,5-dimethylphenyl)amino]pyrimidin-4-yl}-1-methyl-N-(pyridin-2-ylmethyl)-1H-pyrazole-5-carboxamide (cpd 27) [(I), R1=3,5-dimethylphenyl, R2=methyl, R3=pyridin-2-ylmethyl, R4=H]

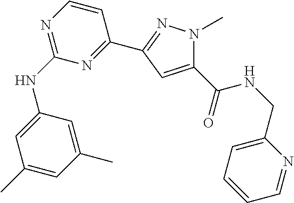 [SMILES: Cc1cc(C)cc(Nc2nccc(-c3cc(C(=O)NCc4ccccn4)n(C)n3)n2)c1]

¹H NMR (500 MHz, DMSO-d6) δ ppm 9.48 (s, 1H), 9.39 (t, J=6.0 Hz, 1H), 8.52 (ddd, J=0.9, 1.6, 4.7 Hz, 1H), 8.50 (d, J=5.0 Hz, 1H), 7.78 (dt, J=1.8, 7.7 Hz, 1H), 7.55 (s, 1H), 7.47 (s, 2H), 7.36 (d, J=7.8 Hz, 1H), 7.29 (ddd, J=0.9, 4.8, 7.4 Hz, 1H), 7.27 (d, J=5.0 Hz, 1H), 6.61 (s, 1H), 4.56 (d, J=5.9 Hz, 2H), 4.16 (s, 3H), 2.26 (s, 6H); HRMS (ESI) calcd for C23H23N7O [M+H]+ 414.2037, found 414.2043.

### 3-{2-[(3,5-dimethylphenyl)amino]pyrimidin-4-yl}-N-(1H-imidazol-2-ylmethyl)-1-methyl-1H-pyrazole-5-carboxamide (cpd 28) [(I), R1=3,5-dimethylphenyl, R2=methyl, R3=1H-imidazol-2-ylmethyl, R4=H]

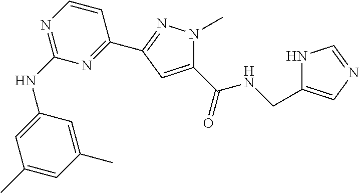 [SMILES: Cc1cc(C)cc(Nc2nccc(-c3cc(C(=O)NCc4cnc[nH]4)n(C)n3)n2)c1]

¹H NMR (500 MHz, DMSO-d6) δ ppm 11.85 (br. s., 1H), 9.46 (s, 1H), 9.24 (t, J=5.7 Hz, 1H), 8.49 (d, J=5.0 Hz, 1H), 7.50 (s, 1H), 7.45 (s, 2H), 7.25 (d, J=5.2 Hz, 1H), 7.03 (s, 1H), 6.82 (s, 1H), 6.60 (s, 1H), 4.47 (d, J=5.6 Hz, 2H), 4.17 (s, 3H), 2.26 (s, 6H); HRMS (ESI) calcd for C21H22N8O [M+H]+ 403.199, found 403.1994.

### N-[(2R)-1-(azetidin-1-yl)propan-2-yl]-3-{2-[(3,5-dimethoxyphenyl)amino]pyrimidin-4-yl}-1-methyl-1H-pyrazole-5-carboxamide (cpd 29) [(I), R1=3,5-dimethoxyphenyl, R2=methyl, R3=(2R)-1-(azetidin-1-yl)propan-2-yl, R4=H]

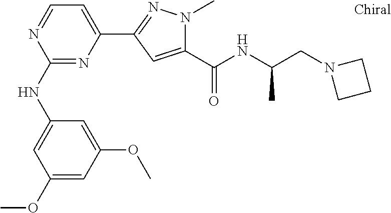 [SMILES: COc1cc(Nc2nccc(-c3cc(C(=O)N[C@H](C)CN4CCC4)n(C)n3)n2)cc(OC)c1]

¹H NMR (500 MHz, DMSO-d6) δ ppm 9.59 (s, 1H), 8.52 (d, J=5.03 Hz, 1H), 8.42 (d, J=8.39 Hz, 1H), 7.45 (s, 1H), 7.28 (d, J=5.03 Hz, 1H), 7.17 (d, J=2.29 Hz, 2H), 6.13 (t, J=2.21 Hz, 1H), 4.12 (s, 3H), 3.90 (d, J=7.93 Hz, 1H), 3.75 (s, 6H), 3.05-3.19 (m, 4H), 2.31-2.48 (m, 2H), 1.94 (t, J=6.94 Hz, 2H), 1.09 (d, J=6.71 Hz, 3H); HRMS (ESI) calcd for C23H29N7O3 [M+H]+ 452.2405, found 452.2407.

### N-[(2S)-1-(dimethylamino)propan-2-yl]-1-methyl-3-[2-(tetrahydro-2H-pyran-4-ylamino)pyrimidin-4-yl]-1H-pyrazole-5-carboxamide (cpd 30) [(I), R1=oxan-4-yl, R2=methyl, R3=(2S)-1-(dimethylamino)propan-2-yl, R4=H]

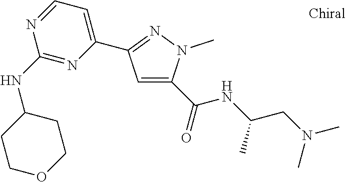 [SMILES: C[C@@H](CN(C)C)NC(=O)c1cc(-c2ccnc(NC3CCOCC3)n2)nn1C]

¹H NMR (500 MHz, DMSO-d6) δ ppm 8.39 (d, J=8.2 Hz, 1H), 8.30 (d, J=5.0 Hz, 1H), 7.37 (s, 1H), 7.14-7.07 (m, 1H), 7.05-7.02 (m, 1H), 4.16-4.07 (m, 4H), 4.01 (br. s., 1H), 3.88 (d, J=11.6 Hz, 2H), 3.40 (t, J=11.2 Hz, 2H), 2.40-2.35 (m, 1H), 2.20 (d, J=6.7 Hz, 1H), 2.16 (s, 6H), 1.84 (d, J=12.4 Hz, 2H), 1.60-1.48 (m, 2H), 1.12 (d, J=6.6 Hz, 3H); HRMS (ESI) calcd for C19H29N7O2 [M+H]+ 388.2456, found 388.2455.

### N-(1-azabicyclo[2.2.2]oct-3-yl)-3-{2-[(3,5-dimethylphenyl)amino]pyrimidin-4-yl}-1-methyl-1H-pyrazole-5-carboxamide (cpd 31) [(I), R1=3,5-dimethylphenyl, R2=methyl, R3=1-azabicyclo[2.2.2]octan-3-yl, R4=H]

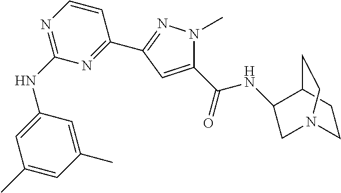 [SMILES: Cc1cc(C)cc(Nc2nccc(-c3cc(C(=O)NC4CN5CCC4CC5)n(C)n3)n2)c1]

¹H NMR (500 MHz, DMSO-d6) δ ppm 9.49 (s, 1H), 8.69 (d, J=6.9 Hz, 1H), 8.49 (d, J=5.0 Hz, 1H), 7.53 (s, 1H), 7.51 (s, 2H), 7.27 (d, J=5.0 Hz, 1H), 6.62 (s, 1H), 4.12 (s, 3H), 3.93 (br. q, J=7.5 Hz, 1H), 3.08 (ddd, J=2.2, 10.2, 13.3 Hz, 1H), 2.86 (dddd, J=2.1, 4.8, 10.8, 13.1 Hz, 1H), 2.75-2.60 (m, 4H), 2.27 (s, 6H), 1.85 (sxt, J=2.8 Hz, 1H), 1.83-1.71 (m, 1H), 1.65-1.50 (m, 2H), 1.32 (dd, J=11.3, 13.6 Hz, 1H); HRMS (ESI) calcd for C24H29N7O [M+H]+ 432.2507, found 432.2505.

### 5-[2-(3,5-Dimethyl-phenylamino)-pyrimidin-4-yl]-2-methyl-2H-pyrazole-3-carboxylic acid ((1R,2R)-2-hydroxy-cyclohexyl)-amide (cpd 32) [(I), R1=3,5-dimethylphenyl, R2=methyl, R3=(1R,2R)-2-hydroxycyclohexyl, R4=H]

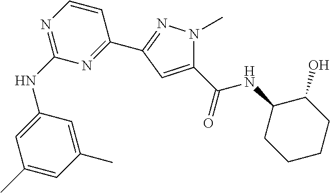 [SMILES: Cc1cc(C)cc(Nc2nccc(-c3cc(C(=O)N[C@@H]4CCCC[C@H]4O)n(C)n3)n2)c1]

¹H NMR (500 MHz, DMSO-d6) δ ppm 9.46 (s, 1H), 8.49 (d, J=5.0 Hz, 1H), 8.46 (d, J=8.4 Hz, 1H), 7.47 (s, 3H), 7.25 (d, J=5.2 Hz, 1H), 6.61 (s, 1H), 4.61 (d, J=5.2 Hz, 1H), 4.13 (s, 3H), 3.66-3.54 (m, 1H), 2.27 (s, 6H), 1.90 (d, J=9.8 Hz, 1H), 1.83 (d, J=8.8 Hz, 1H), 1.65 (d, J=8.5 Hz, 2H), 1.33-1.10 (m, 4H); HRMS (ESI) calcd for C23H28N6O2 [M+H]+ 421.2347, found 421.2349.

### 3-{2-[(3,5-dimethylphenyl)amino]pyrimidin-4-yl}-N-[(2S)-1-hydroxybutan-2-yl]-1-methyl-1H-pyrazole-5-carboxamide (cpd 33) [(I), R1=3,5-dimethylphenyl, R2=methyl, R3=(2S)-1-hydroxybutan-2-yl, R4=H]

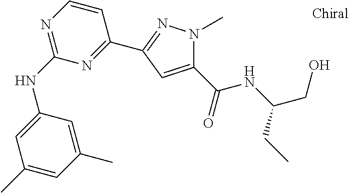 [SMILES: CC[C@@H](CO)NC(=O)c1cc(-c2ccnc(Nc3cc(C)cc(C)c3)n2)nn1C]

¹H NMR (500 MHz, DMSO-d6) δ ppm 9.47 (s, 1H), 8.49 (d, J=5.0 Hz, 1H), 8.38 (d, J=8.7 Hz, 1H), 7.49 (s, 1H), 7.48 (s, 2H), 7.25 (d, J=5.0 Hz, 1H), 6.61 (s, 1H), 4.71 (t, J=5.6 Hz, 1H), 4.13 (s, 3H), 3.90-3.75 (m, 1H), 2.26 (s, 6H), 1.74-1.58 (m, 1H), 1.51-1.38 (m, 1H), 0.88 (t, J=7.5 Hz, 3H); HRMS (ESI) calcd for C21H26N6O2 [M+H]+ 395.219, found 395.2185.

### 3-{2-[(3,5-dimethylphenyl)amino]pyrimidin-4-yl}-N-(2-hydroxyethyl)-1-methyl-1H-pyrazole-5-carboxamide (cpd 34) [(I), R1=3,5-dimethylphenyl, R2=methyl, R3=2-hydroxyethyl, R4=H]

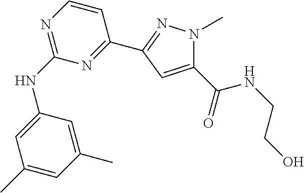 [SMILES: Cc1cc(C)cc(Nc2nccc(-c3cc(C(=O)NCCO)n(C)n3)n2)c1]

¹H NMR (500 MHz, DMSO-d6) δ ppm 9.45 (s, 1H), 8.71 (t, J=5.6 Hz, 1H), 8.49 (d, J=5.0 Hz, 1H), 7.46 (s, 2H), 7.44 (s, 1H), 7.25 (d, J=5.0 Hz, 1H), 6.61 (s, 1H), 4.76 (t, J=5.7 Hz, 1H), 4.13 (s, 3H), 3.51 (q, J=6.0 Hz, 2H), 3.31 (q, J=6.0 Hz, 2H), 2.27 (s, 6H); HRMS (ESI) calcd for C19H22N6O2 [M+H]+ 367.1877, found 367.1872.

### N-[(2S)-1-(azetidin-1-yl)propan-2-yl]-3-{2-[(3,4-dimethoxyphenyl)amino]pyrimidin-4-yl}-1-methyl-1H-pyrazole-5-carboxamide (cpd 35) [(I), R1=3,4-dimethoxyphenyl, R2=methyl, R3=(2S)-1-(azetidin-1-yl)propan-2-yl, R4=H]

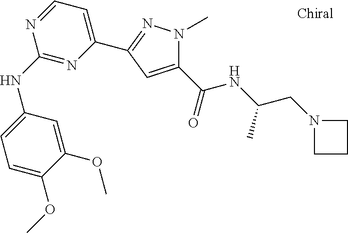 [SMILES: COc1ccc(Nc2nccc(-c3cc(C(=O)N[C@@H](C)CN4CCC4)n(C)n3)n2)cc1OC]

¹H NMR (500 MHz, DMSO-d6) δ ppm 9.44 (s, 1H), 8.47 (d, J=5.0 Hz, 1H), 8.42 (d, J=8.2 Hz, 1H), 7.68 (d, J=1.8 Hz, 1H), 7.46 (s, 1H), 7.23 (dd, J=2.4, 8.5 Hz, 2H), 6.89 (d, J=8.8 Hz, 1H), 4.13 (s, 3H), 3.91 (td, J=7.0, 14.4 Hz, 1H), 3.79 (s, 3H), 3.72 (s, 3H), 3.22-3.01 (m, 4H), 2.55-2.35 (m. Partially overlapped with water, 2H), 1.95 (quin, J=6.8 Hz, 2H), 1.10 (d, J=6.7 Hz, 3H); HRMS (ESI) calcd for C23H29N7O3 [M+H]+ 452.2405, found 452.2413.

### (3-{2-[(3,5-dimethylphenyl)amino]pyrimidin-4-yl}-1-methyl-1H-pyrazol-5-yl)(piperazin-1-yl)methanone hydrochloride (cpd 36) [(I), R1=3,5-dimethylphenyl, R2=methyl, NR3R4=piperazin-1-ylamino]

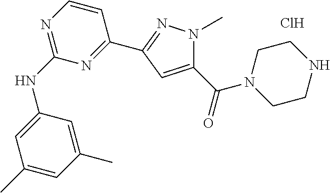 [SMILES: Cc1cc(C)cc(Nc2nccc(-c3cc(C(=O)N4CCNCC4)n(C)n3)n2)c1 ; Cl]

¹H NMR (500 MHz, DMSO-d6) δ ppm 9.53 (s, 1H), 9.13 (br. s., 2H), 8.50 (d, J=5.0 Hz, 1H), 7.47 (s, 2H), 7.28 (d, J=5.0 Hz, 1H), 7.08 (s, 1H), 6.61 (s, 1H), 3.98 (s, 3H), 3.84 (br. s., 4H), 3.21 (br. s., 4H), 2.26 (s, 6H); HRMS (ESI) calcd for C21H26N7O Cl [M+H]+ 392.2194, found 392.2207.

### N-[(1S,2R)-2-aminocyclohexyl]-3-(2-{[3-methoxy-5-(trifluoromethyl)phenyl]amino}pyrimidin-4-yl)-1-methyl-1H-pyrazole-5-carboxamide hydrochloride (cpd 37) [(I), R1=3-methoxy-5-(trifluoromethyl)phenyl, R2=methyl, R3=(1S,2R)-2-aminocyclohexyl, R4=H]

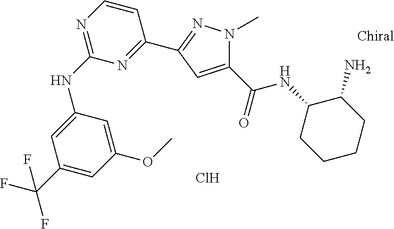 [SMILES: COc1cc(Nc2nccc(-c3cc(C(=O)N[C@H]4CCCC[C@H]4N)n(C)n3)n2)cc(C(F)(F)F)c1 ; Cl]

¹H NMR (500 MHz, DMSO-d6) δ ppm 10.03 (s, 1H), 8.59 (d, J=5.2 Hz, 1H), 8.39 (d, J=7.9 Hz, 1H), 7.97 (s, 1H), 7.88 (br. s., 3H), 7.82 (t, J=1.8 Hz, 1H), 7.57 (s, 1H), 7.37 (d, J=5.0 Hz, 1H), 6.83 (s, 1H), 4.33 (br. s., 1H), 4.14 (s, 3H), 3.85 (s, 3H), 1.87-1.56 (m, 6H), 1.52-1.32 (m, 2H); HRMS (ESI) calcd for C23H27N7O2F3Cl [M+H]+ 490.2173, found 490.2173.

### N-[(1S,2R)-2-aminocyclohexyl]-3-{2-[(3-chloro-1-methyl-1H-indol-5-yl)amino]pyrimidin-4-yl}-1-methyl-1H-pyrazole-5-carboxamide hydrochloride (cpd 38) [(I), R1=3-chloro-1-methylindol-5-yl, R2=methyl, R3=(1S,2R)-2-aminocyclohexyl, R4=H]

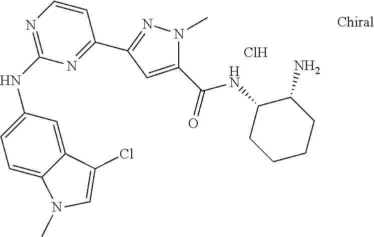 [SMILES: Cn1nc(-c2ccnc(Nc3ccc4c(c3)c(Cl)cn4C)n2)cc1C(=O)N[C@H]1CCCC[C@H]1N ; Cl]

¹H NMR (500 MHz, DMSO-d6) δ ppm 9.58 (s, 1H), 8.50 (d, J=5.0 Hz, 1H), 8.45 (d, J=7.8 Hz, 1H), 8.34 (br. s., 1H), 7.88 (br. s., 3H), 7.59 (s, 1H), 7.51-7.45 (m, 2H), 7.45-7.40 (m, 1H), 7.27 (d, J=5.0 Hz, 1H), 4.32 (br. s., 1 H), 4.14 (s, 3H), 3.76 (s, 3H), 3.42 (s, 1H), 1.88-1.56 (m, 6H), 1.53-1.32 (m, 2H); HRMS (ESI) calcd for C24H28N8O Cl2 [M+H]+ 479.2069, found 479.2074.

### N-[(2S)-1-(azetidin-1-yl)propan-2-yl]-3-{2-[(3-methoxyphenyl)amino]pyrimidin-4-yl}-1-methyl-1H-pyrazole-5-carboxamide (cpd 39) [(I), R1=3-methoxyphenyl, R2=methyl, R3=(2S)-1-(azetidin-1-yl)propan-2-yl, R4=H]

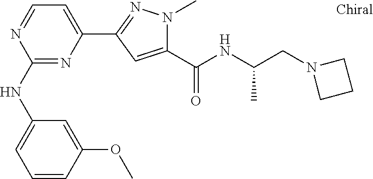 [SMILES: COc1cccc(Nc2nccc(-c3cc(C(=O)N[C@@H](C)CN4CCC4)n(C)n3)n2)c1]

¹H NMR (500 MHz, DMSO-d6) δ ppm 9.63 (s, 1H), 8.51 (d, J=5.0 Hz, 1H), 8.44 (d, J=8.4 Hz, 1H), 7.65-7.59 (m, 1H), 7.46 (s, 1H), 7.38 (dd, J=1.1, 8.2 Hz, 1H), 7.28 (d, J=5.0 Hz, 1H), 7.19 (t, J=8.1 Hz, 1H), 6.54 (dd, J=2.1, 7.8 Hz, 1H), 4.13 (s, 3H), 3.99-3.87 (m, 1H), 3.77 (s, 3H), 3.16 (d, J=3.4 Hz, 4H), 2.55-2.35 (m. Partially overlapped with water, 2H) 2.07-1.88 (m, 2H), 1.11 (d, J=6.7 Hz, 3H); HRMS (ESI) calcd for C22H27N7O2 [M+H]+ 422.2299, found 422.2306.

### N-[(2S)-1-(azetidin-1-yl)propan-2-yl]-3-{2-[(3,5-difluorophenyl)amino]pyrimidin-4-yl}-1-methyl-1H-pyrazole-5-carboxamide (cpd 40) [(I), R1=3,5-difluorophenyl, R2=methyl, R3=(2S)-1-(azetidin-1-yl)propan-2-yl, R4=H]

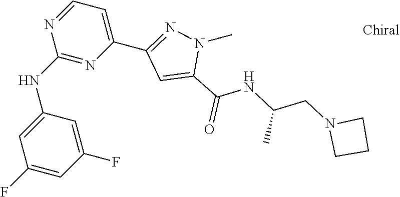 [SMILES: C[C@@H](CN1CCC1)NC(=O)c1cc(-c2ccnc(Nc3cc(F)cc(F)c3)n2)nn1C]

¹H NMR (500 MHz, DMSO-d6) δ ppm 10.10 (s, 1H), 8.59 (d, J=5.2 Hz, 1H), 8.45 (d, J=8.2 Hz, 1H), 7.67-7.57 (m, 2H), 7.45 (s, 1H), 7.38 (d, J=5.2 Hz, 1H), 6.76 (tt, J=2.3, 9.3 Hz, 1H), 4.14 (s, 3H), 3.96-3.86 (m, 1H), 3.24-3.03 (m, 4H), 2.49-2.36 (m, 2H), 1.94 (quin, J=6.9 Hz, 2H), 1.16-1.04 (m, 3H); HRMS (ESI) calcd for C21H23N7O F2 [M+H]+ 428.2005, found 428.2006.

### N-[(2S)-1-hydroxy-3-methylbutan-2-yl]-1-methyl-3-{2-[(3-methylphenyl)amino]pyrimidin-4-yl}-1H-pyrazole-5-carboxamide (cpd 41) [(I), R1=3-methylphenyl, R2=methyl, R3=(2S)-1-hydroxy-3-methylbutan-2-yl, R4=H]

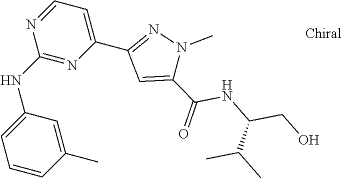 [SMILES: Cc1cccc(Nc2nccc(-c3cc(C(=O)N[C@H](CO)C(C)C)n(C)n3)n2)c1]

¹H NMR (500 MHz, DMSO-d6) δ ppm 9.56 (s, 1H), 8.50 (d, J=5.0 Hz, 1H), 8.35 (d, J=9.0 Hz, 1H), 7.75 (s, 1H), 7.59 (d, J=8.5 Hz, 1H), 7.52 (s, 1H), 7.28 (d, J=5.0 Hz, 1H), 7.17 (t, J=7.8 Hz, 1H), 6.78 (d, J=7.5 Hz, 1H), 4.60 (t, J=5.6 Hz, 1H), 4.13 (s, 3H), 3.84-3.74 (m, 1H), 3.59-3.44 (m, 2H), 2.31 (s, 3H), 1.91 (d, J=6.9 Hz, 1H), 0.91 (dd, J=6.9, 10.2 Hz, 6H); HRMS (ESI) calcd for C21H26N6O2 [M+H]+ 395.219, found 395.2196.

### 3-{2-[(3,5-dimethylphenyl)amino]pyrimidin-4-yl}-1-methyl-N-[(2S)-1,1,1-trifluoropropan-2-yl]-1H-pyrazole-5-carboxamide (cpd 42) [(I), R1=3,5-dimethylphenyl, R2=methyl, R3=(2S)-1,1,1-trifluoropropan-2-yl, R4=H]

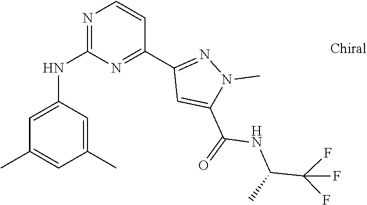 [SMILES: Cc1cc(C)cc(Nc2nccc(-c3cc(C(=O)N[C@@H](C)C(F)(F)F)n(C)n3)n2)c1]

¹H NMR (500 MHz, DMSO-d6) δ ppm 9.49 (s, 1H), 9.23 (d, J=8.7 Hz, 1H), 8.51 (d, J=5.0 Hz, 1H), 7.60 (s, 1H), 7.48 (s, 2H), 7.26 (d, J=5.0 Hz, 1H), 6.62 (s, 1H), 4.81 (qd, J=7.6, 15.3 Hz, 1H), 4.14 (s, 3H), 2.27 (s, 6H), 1.36 (d, J=7.2 Hz, 3H); HRMS (ESI) calcd for C20H21N6O F3 [M+H]+ 419.1802, found 419.1813.

### N-[3-(dimethylamino)propyl]-3-{2-[(3,5-dimethylphenyl)amino]pyrimidin-4-yl}-1-methyl-1H-pyrazole-5-carboxamide (cpd 43) [(I), R1=3,5-dimethylphenyl, R2=methyl, R3=3-(dimethylamino)propyl, R4=H]

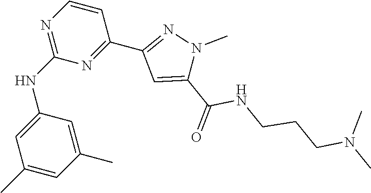

¹H NMR (500 MHz, DMSO-d6) δ ppm 9.46 (s, 1H), 8.78 (t, J=5.6 Hz, 1H), 8.49 (d, J=5.0 Hz, 1H), 7.47 (s, 2H), 7.25 (d, J=5.0 Hz, 1H), 6.61 (s, 1H), 4.13 (s, 3H), 3.29-3.23 (m, 2H), 2.27 (s, 6H), 2.26-2.22 (m, 2H), 2.13 (s, 6H), 1.64 (quin, J=7.0 Hz, 2H); HRMS (ESI) calcd for C22H29N7O [M+H]+ 408.2507, found 408.2499.

### (3-{2-[(3,5-dimethylphenyl)amino]pyrimidin-4-yl}-1-methyl-1H-pyrazol-5-yl)[(2S)-2-(propan-2-yl)aziridin-1-yl]methanone (cpd 44) [(I), R1=3,5-dimethylphenyl, R2=methyl, NR3R4=(1R,2S)-2-propan-2-ylaziridin-1-ylamino]

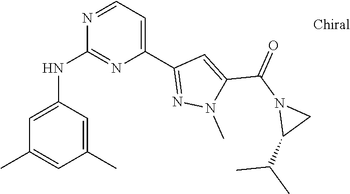 [SMILES: Cc1cc(C)cc(Nc2nccc(-c3cc(C(=O)N4C[C@@H]4C(C)C)n(C)n3)n2)c1]

¹H NMR (500 MHz, DMSO-d6) δ ppm 9.51 (s, 1H), 8.50 (d, J=5.2 Hz, 1H), 7.47 (s, 2H), 7.28 (d, J=5.0 Hz, 1H), 7.26 (s, 1H), 6.61 (s, 1H), 4.49-4.39 (m, 1H), 4.23 (s, 3H), 4.18-4.10 (m, 2H), 2.25 (s, 6H), 1.78 (qd, J=6.6, 13.2 Hz, 1H), 0.97 (d, J=6.7 Hz, 3H), 0.91 (d, J=6.7 Hz, 3H); HRMS (ESI) calcd for C22H26N6O [M+H]+391.2241, found 391.2246.

### 3-{2-[(3,5-dimethylphenyl)amino]pyrimidin-4-yl}-N-(2,2-dimethylpropyl)-1-methyl-1H-pyrazole-5-carboxamide (cpd 45) [(I), R1=3,5-dimethylphenyl, R2=methyl, R3=2,2-dimethylpropyl, R4=H]

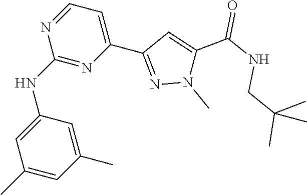 [SMILES: Cc1cc(C)cc(Nc2nccc(-c3cc(C(=O)NCC(C)(C)C)n(C)n3)n2)c1]

¹H NMR (500 MHz, DMSO-d6) δ ppm 9.49 (s, 1H), 8.72 (t, J=6.3 Hz, 1H), 8.49 (d, J=5.0 Hz, 1H), 7.49 (s, 2H), 7.27 (d, J=5.0 Hz, 1H), 6.61 (s, 1H), 4.12 (s, 3H), 3.08 (d, J=6.4 Hz, 2H), 2.26 (s, 6H), 0.91 (s, 9H); HRMS (ESI) calcd for C22H28N6O [M+H]+ 393.2398, found 393.2399.

### 3-{2-[(3,5-dimethylphenyl)amino]pyrimidin-4-yl}-1-methyl-N-(2-methylpropyl)-1H-pyrazole-5-carboxamide (cpd 46) [(I), R1=3,5-dimethylphenyl, R2=methyl, R3=2-methylpropyl, R4=H]

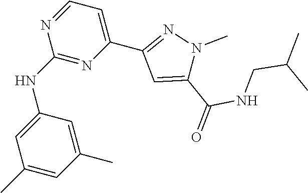 [SMILES: Cc1cc(C)cc(Nc2nccc(-c3cc(C(=O)NCC(C)C)n(C)n3)n2)c1]

¹H NMR (500 MHz, DMSO-d6) δ ppm 9.48 (s, 1H), 8.78 (t, J=5.9 Hz, 1H), 8.49 (d, J=5.0 Hz, 1H), 7.48 (s, 2H), 7.44 (s, 1H), 7.26 (d, J=5.0 Hz, 1H), 6.61 (s, 1H), 4.13 (s, 3H), 3.07 (t, J=6.4 Hz, 2H), 2.27 (s, 6H), 1.84 (quind, J=6.7, 13.5 Hz, 1H), 0.90 (d, J=6.7 Hz, 6H); HRMS (ESI) calcd for C21H26N6O [M+H]+ 379.2241, found 379.2254.

### N-(cyclopropylmethyl)-3-{2-[(3,5-dimethylphenyl)amino]pyrimidin-4-yl}-1-methyl-1H-pyrazole-5-carboxamide (cpd 47) [(I), R1=3,5-dimethylphenyl, R2=methyl, R3=cyclopropylmethyl, R4=H]

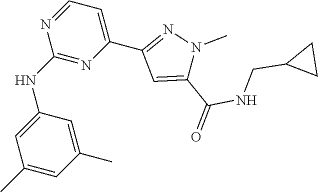 [SMILES: Cc1cc(C)cc(Nc2nccc(-c3cc(C(=O)NCC4CC4)n(C)n3)n2)c1]

¹H NMR (500 MHz, DMSO-d6) δ ppm 9.47 (s, 1H), 8.87 (t, J=5.7 Hz, 1H), 8.49 (d, J=5.0 Hz, 1H), 7.48 (s, 2H), 7.46 (s, 1H), 7.26 (d, J=5.0 Hz, 1H), 6.62 (s, 1H), 4.14 (s, 3H), 3.13 (t, J=6.3 Hz, 2H), 2.27 (s, 6H), 1.09-0.95 (m, 1H), 0.52-0.37 (m, 2H), 0.29-0.18 (m, 2H); HRMS (ESI) calcd for C21H24N6O [M+H]+ 377.2085, found 377.209.

### N-[(2S)-1-(azetidin-1-yl)propan-2-yl]-3-{2-[(3-fluoro-5-methoxyphenyl)amino]pyrimidin-4-yl}-1-methyl-1H-pyrazole-5-carboxamide (cpd 48) [(I), R1=3-fluoro-5-methoxyphenyl, R2=methyl, R3=(2S)-1-(azetidin-1-yl)propan-2-yl, R4=H]

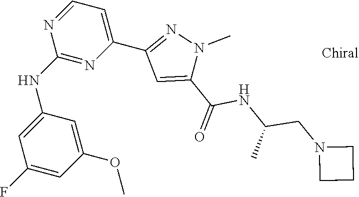 [SMILES: COc1cc(F)cc(Nc2nccc(-c3cc(C(=O)N[C@@H](C)CN4CCC4)n(C)n3)n2)c1]

¹H NMR (500 MHz, DMSO-d6) δ ppm 9.84 (s, 1H), 8.56 (d, J=5.2 Hz, 1H), 8.47 (d, J=6.6 Hz, 1H), 7.45 (s, 1H), 7.39 (s, 1H), 7.38-7.34 (m, 1H), 7.33 (d, J=5.0 Hz, 1H), 6.41 (td, J=2.3, 10.8 Hz, 1H), 4.14 (s, 3H), 4.03-3.88 (m, 1H), 3.78 (s, 3H), 3.30-3.00 (m, 4H), 2.54-2.38 (m. partially overlapped with water, 2H) 2.12-1.89 (m, 2H), 1.11 (d, J=6.9 Hz, 3H); HRMS (ESI) calcd for C22H26N7O2F [M+H]+ 440.2205, found 440.2202.

### 3-{2-[(4,6-dimethylpyridin-2-yl)amino]pyrimidin-4-yl}-N-[(2S)-1-hydroxy-3-methylbutan-2-yl]-1-methyl-1H-pyrazole-5-carboxamide (cpd 49) [(I), R1=4,6-dimethylpyridin-2-yl, R2=methyl, R3=(2S)-1-hydroxy-3-methylbutan-2-yl, R4=H]

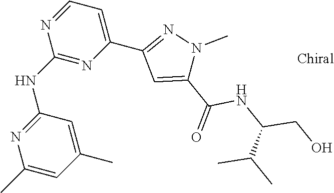 [SMILES: Cc1cc(C)nc(Nc2nccc(-c3cc(C(=O)N[C@H](CO)C(C)C)n(C)n3)n2)c1]

¹H NMR (500 MHz, DMSO-d6) δ ppm 9.44 (s, 1H), 8.56 (d, J=5.0 Hz, 1H), 8.38 (d, J=9.0 Hz, 1H), 8.12 (s, 1H), 7.55 (s, 1H), 7.37 (d, J=5.0 Hz, 1H), 6.73 (s, 1H), 4.60 (br. s., 1H), 4.13 (s, 3H), 3.87-3.72 (m, 1H), 3.59-3.43 (m, 2H), 2.36 (s, 3H), 2.33 (s, 3H), 1.92 (qd, J=6.7, 13.5 Hz, 1H), 0.91 (dd, J=6.8, 9.5 Hz, 6H); HRMS (ESI) calcd for C21H27N7O2 [M+H]+ 410.2299, found 410.2301.

### 3-{2-[(3,5-difluorophenyl)amino]pyrimidin-4-yl}-1-methyl-N-[(2S)-1,1,1-trifluoropropan-2-yl]-1H-pyrazole-5-carboxamide (cpd 50) [(I), R1=3,5-difluorophenyl, R2=methyl, R3=(2S)-1,1,1-trifluoropropan-2-yl, R4=H]

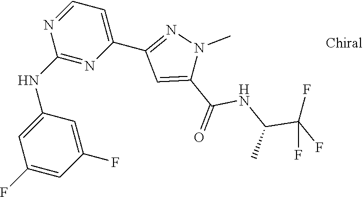 [SMILES: C[C@H](NC(=O)c1cc(-c2ccnc(Nc3cc(F)cc(F)c3)n2)nn1C)C(F)(F)F]

¹H NMR (500 MHz, DMSO-d6) δ ppm 10.12 (s, 1H), 9.20 (d, J=8.7 Hz, 1H), 8.61 (d, J=5.2 Hz, 1H), 7.68-7.57 (m, 3H), 7.39 (d, J=5.0 Hz, 1H), 6.81-6.70 (m, 1H), 4.90-4.73 (m, 1H), 4.15 (s, 3H), 1.37 (d, J=7.2 Hz, 3H); HRMS (ESI) calcd for C18H15N6O F5 [M+H]+ 427.13, found 427.1308.

### 1-methyl-3-{2-[(3-methylphenyl)amino]pyrimidin-4-yl}-N-[(2S)-1,1,1-trifluoropropan-2-yl]-1H-pyrazole-5-carboxamide (cpd 51) [(I), R1=3-methylphenyl, R2=methyl, R3=(2S)-1,1,1-trifluoropropan-2-yl, R4=H]

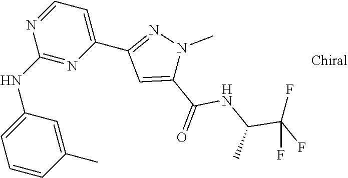 [SMILES: Cc1cccc(Nc2nccc(-c3cc(C(=O)N[C@@H](C)C(F)(F)F)n(C)n3)n2)c1]

¹H NMR (500 MHz, DMSO-d6) δ ppm 9.57 (s, 1H), 9.22 (d, J=8.7 Hz, 1H), 8.51 (d, J=5.2 Hz, 1H), 7.72 (s, 1H), 7.64-7.57 (m, 2H), 7.28 (d, J=5.2 Hz, 1H), 7.18 (t, J=7.9 Hz, 1H), 6.79 (d, J=7.5 Hz, 1H), 4.89-4.73 (m, 1H), 4.15 (s, 3H), 2.31 (s, 3H), 1.37 (d, J=7.0 Hz, 3H); HRMS (ESI) calcd for C19H19N6O F3 [M+H]+ 405.1645, found 405.1649.

### 3-(2-{[3-chloro-4-(4-methylpiperazin-1-yl)phenyl]amino}pyrimidin-4-yl)-N-[(2S)-1-hydroxy-3-phenylpropan-2-yl]-1-methyl-1H-pyrazole-5-carboxamide (cpd 52) [(I), R1=3-chloro-4-(4-methylpiperazin-1-yl)phenyl, R2=methyl, R3=(2S)-1-hydroxy-3-phenylpropan-2-yl, R4=H]

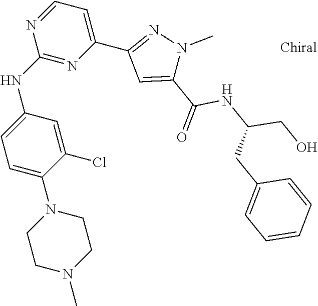 [SMILES: CN1CCN(c2ccc(Nc3nccc(-c4cc(C(=O)N[C@H](CO)Cc5ccccc5)n(C)n4)n3)cc2Cl)CC1]

¹H NMR (500 MHz, DMSO-d6) δ ppm 9.68 (s, 1H), 8.50 (d, J=5.2 Hz, 1H), 8.50 (d, J=8.2 Hz, 1H), 7.92 (d, J=2.4 Hz, 1H), 7.82 (dd, J=2.0, 8.8 Hz, 1H), 7.42 (s, 1H), 7.32-7.22 (m, 5H), 7.16 (d, J=8.8 Hz, 1H), 7.21-7.11 (m, 1H), 4.89 (t, J=5.7 Hz, 1H), 4.17-4.07 (m, 1H), 4.03 (s, 3H), 3.56-3.48 (m, 1H), 3.48-3.40 (m, 1H), 2.96 (dd, J=4.7, 13.9 Hz, 1H), 2.94 (br. s, 4H), 2.75 (dd, J=9.7, 13.7 Hz, 1H), 2.48 (br. s., 4H), 2.23 (s, 3H); HRMS (ESI) calcd for C29H33N8O2 Cl [M+H]+ 561.2488, found 561.2498.

### N-[(1S,2S)-2-aminocyclohexyl]-1-methyl-3-{2-[(3-methylphenyl)amino]pyrimidin-4-yl}-1H-pyrazole-5-carboxamide (cpd 53) [(1), R1=3-methylphenyl, R2=methyl, R3=(1S,2S)-2-aminocyclohexyl, R4=H]

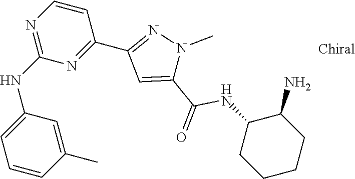

¹H NMR (500 MHz, DMSO-d6) δ ppm 9.55 (s, 1H), 8.54-8.44 (m, 2H), 7.69 (s, 1H), 7.63 (d, J=8.2 Hz, 1H), 7.51-7.48 (m, 1H), 7.27 (d, J=5.0 Hz, 1H), 7.19 (t, J=7.8 Hz, 1H), 6.79 (d, J=7.5 Hz, 1H), 4.16-4.10 (m, 3H), 3.45 (d, J=5.2 Hz, 1H), 2.60-2.53 (m, 1H), 2.31 (s, 3H), 1.91-1.80 (m, 2H), 1.66 (br. s., 3H), 1.38-1.02 (m, 5H); HRMS (ESI) calcd for C22H27N7O [M+H]+ 406.235, found 406.2348.

### 3-(2-{[3-chloro-4-(4-methylpiperazin-1-yl)phenyl]amino}pyrimidin-4-yl)-1-methyl-N-[(2S)-1,1,1-trifluoropropan-2-yl]-1H-pyrazole-5-carboxamide (cpd 54) [(I), R1=3-chloro-4-(4-methylpiperazin-1-yl)phenyl, R2=methyl, R3=(2S)-1,1,1-trifluoropropan-2-yl, R4=H]

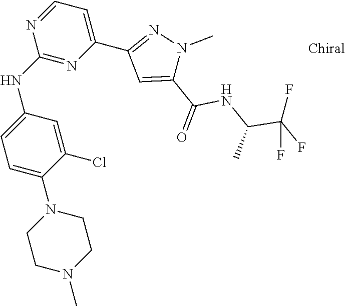 [SMILES: C[C@H](NC(=O)c1cc(-c2ccnc(Nc3ccc(N4CCN(C)CC4)c(Cl)c3)n2)nn1C)C(F)(F)F]

¹H NMR (500 MHz, DMSO-d6) δ ppm 9.69 (s, 1H), 9.19 (d, J=8.8 Hz, 1H), 8.52 (d, J=5.0 Hz, 1H), 7.99 (d, J=2.4 Hz, 1H), 7.71 (dd, J=2.5, 8.8 Hz, 1H), 7.58 (s, 1H), 7.29 (d, J=5.2 Hz, 1H), 7.13 (d, J=8.8 Hz, 1H), 4.82 (qd, J=7.6, 15.4 Hz, 1H), 4.14 (s, 3H), 2.93 (br. s., 4H), 2.47 (br. s., 4H), 2.23 (s, 3H), 1.37 (d, J=7.2 Hz, 3H); HRMS (ESI) calcd for C23H26N8O F3Cl [M+H]+ 523.1943, found 523.1951.

### 3-{2-[(3,5-dimethylphenyl)amino]pyrimidin-4-yl}-1-methyl-N-[(2S)-1,1,1-trifluorobutan-2-yl]-1H-pyrazole-5-carboxamide (cpd 55) [(I), R1=3,5-dimethylphenyl, R2=methyl, R3=(2S)-1,1,1-trifluorobutan-2-yl, R4=H]

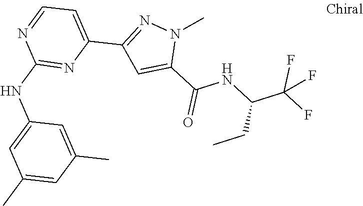 [SMILES: CC[C@H](NC(=O)c1cc(-c2ccnc(Nc3cc(C)cc(C)c3)n2)nn1C)C(F)(F)F]

¹H NMR (500 MHz, DMSO-d6) δ ppm 9.51 (s, 1H), 9.14 (d, J=9.0 Hz, 1H), 8.51 (d, J=5.0 Hz, 1H), 7.63 (s, 1H), 7.49 (s, 2H), 7.27 (d, J=5.2 Hz, 1H), 6.62 (s, 1H), 4.68-4.50 (m, 1H), 4.15 (s, 3H), 2.27 (s, 6H), 1.89-1.66 (m, 2H), 0.96 (t, J=7.3 Hz, 3H); HRMS (ESI) calcd for C21H23N6O F3 [M+H]+ 433.1958, found 433.1959.

### 3-{2-[(3,5-dimethylphenyl)amino]pyrimidin-4-yl}-1-methyl-N-[(2R)-1,1,1-trifluorobutan-2-yl]-1H-pyrazole-5-carboxamide (cpd 56) [(I), R1=3,5-dimethylphenyl, R2=methyl, R3=(2R)-1,1,1-trifluorobutan-2-yl, R4=H]

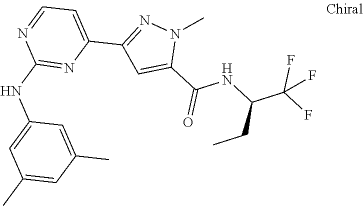 [SMILES: CC[C@@H](NC(=O)c1cc(-c2ccnc(Nc3cc(C)cc(C)c3)n2)nn1C)C(F)(F)F]

¹H NMR (500 MHz, DMSO-d6) δ ppm 9.51 (s, 1H), 9.14 (d, J=9.0 Hz, 1H), 8.51 (d, J=5.0 Hz, 1H), 7.63 (s, 1H), 7.49 (s, 2H), 7.27 (d, J=5.2 Hz, 1H), 6.62 (s, 1H), 4.68-4.49 (m, 1H), 4.15 (s, 3H), 2.27 (s, 6H), 1.88-1.64 (m, 2H), 0.96 (t, J=7.4 Hz, 3H); HRMS (ESI) calcd for C21H23N6O F3 [M+H]+ 433.1958, found 433.1957.

### N-[(2S)-1-(3,3-difluoroazetidin-1-yl)propan-2-yl]-3-{2-[(3,5-dimethylphenyl)amino]pyrimidin-4-yl}-1-methyl-1H-pyrazole-5-carboxamide (cpd 57) [(I), R1=3,5-dimethylphenyl, R2=methyl, R3=(2S)-1-(3,3-difluoroazetidin-1-yl)propan-2-yl, R4=H]

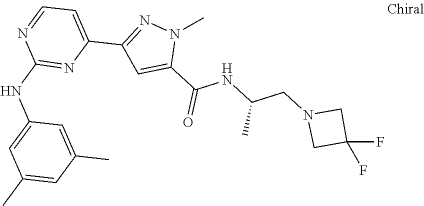 [SMILES: Cc1cc(C)cc(Nc2nccc(-c3cc(C(=O)N[C@@H](C)CN4CC(F)(F)C4)n(C)n3)n2)c1]

¹H NMR (500 MHz, DMSO-d6) δ ppm 9.47 (s, 1H), 8.54 (d, J=8.4 Hz, 1H), 8.49 (d, J=5.2 Hz, 1H), 7.48 (s, 2H), 7.45 (s, 1H), 7.26 (d, J=5.0 Hz, 1H), 6.62 (s, 1H), 4.13 (s, 3H), 3.98 (quind, J=6.9, 13.9 Hz, 1H), 3.73-3.53 (m, 4H), 2.70-2.56 (m, 2H), 2.27 (s, 6H), 1.13 (d, J=6.7 Hz, 3H); HRMS (ESI) calcd for C23H27N7O F2 [M+H]+456.2318, found 456.232.

### 1-methyl-N-[(2S)-1-(pyrrolidin-1-yl)propan-2-yl]-3-{2-[(3,4,5-trimethoxyphenyl)amino]pyrimidin-4-yl}-1H-pyrazole-5-carboxamide (cpd 58) [(I), R1=3,4,5-trimethoxyphenyl, R2=methyl, R3=(2S)-1-pyrrolidin-1-ylpropan-2-yl, R4=H]

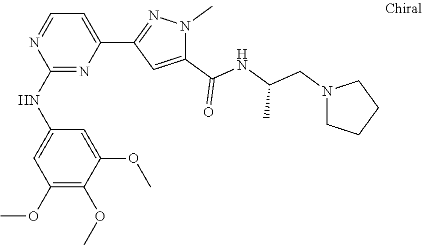 [SMILES: COc1cc(Nc2nccc(-c3cc(C(=O)N[C@@H](C)CN4CCCC4)n(C)n3)n2)cc(OC)c1OC]

¹H NMR (500 MHz, DMSO-d6) δ ppm 9.53 (s, 1H), 8.51 (d, J=5.03 Hz, 2H), 7.50 (s, 1H), 7.31 (s, 2H), 7.26 (d, J=5.19 Hz, 1H), 4.07-4.20 (m, 4H), 3.80 (s, 6H), 3.63 (s, 3H), 2.60-2.38 (m. Partially overlapped with water, 6H), 1.69 (br. s., 4H), 1.08-1.20 (m, 3H); HRMS (ESI) calcd for C25H33N7O4 [M+H]+ 496.2667, found 496.2666.

### N-[(2S)-1-(azetidin-1-yl)propan-2-yl]-3-{2-[(3-chloro-1-methyl-1H-indol-5-yl)amino]pyrimidin-4-yl}-1-methyl-1H-pyrazole-5-carboxamide (cpd 59) [(I), R1=3-chloro-1-methylindol-5-yl, R2=methyl, R3=(2S)-1-(azetidin-1-yl)propan-2-yl, R4=H]

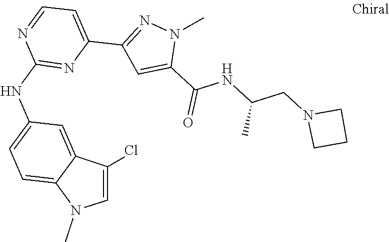 [SMILES: C[C@@H](CN1CCC1)NC(=O)c1cc(-c2ccnc(Nc3ccc4c(c3)c(Cl)cn4C)n2)nn1C]

¹H NMR (500 MHz, DMSO-d6) δ ppm 9.56 (s, 1H), 8.49 (d, J=5.0 Hz, 1H), 8.41 (d, J=8.4 Hz, 1H), 8.28 (s, 1H), 7.51 (dd, J=2.0, 9.0 Hz, 1H), 7.47 (s, 1H), 7.44 (s, 1H), 7.42 (d, J=8.8 Hz, 1H), 7.24 (d, J=5.0 Hz, 1H), 4.12 (s, 3H), 3.97-3.84 (m, 1H), 3.76 (s, 3H), 3.23-3.05 (m, 4H), 2.46 (dd, J=6.9, 11.6 Hz, 1H), 2.37 (dd, J=6.7, 11.6 Hz, 1H), 1.95 (quin, J=6.9 Hz, 2H), 1.11 (d, J=6.7 Hz, 3H); HRMS (ESI) calcd for C24H27N8O Cl [M+H]+479.2069, found 479.2068.

### N-[(2S)-1-(azetidin-1-yl)propan-2-yl]-3-{2-[(3,5-dichlorophenyl)amino]pyrimidin-4-yl}-1-methyl-1H-pyrazole-5-carboxamide (cpd 60) [(I), R1=3,5-dichlorophenyl, R2=methyl, R3=(2S)-1-(azetidin-1-yl)propan-2-yl, R4=H]

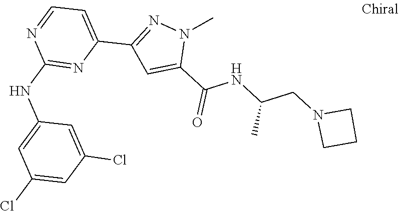 [SMILES: C[C@@H](CN1CCC1)NC(=O)c1cc(-c2ccnc(Nc3cc(Cl)cc(Cl)c3)n2)nn1C]

¹H NMR (500 MHz, DMSO-d6) δ ppm 10.08 (s, 1H), 8.60 (d, J=5.2 Hz, 1H), 8.45 (d, J=8.4 Hz, 1H), 7.98 (d, J=2.0 Hz, 2H), 7.43 (s, 1H), 7.38 (d, J=5.0 Hz, 1H), 7.14 (t, J=1.8 Hz, 1H), 4.14 (s, 3H), 3.93 (br. s., 1H), 3.17 (br. s, 4H), 2.42 (br. s., 2H), 1.96 (br. s, 2H), 1.10 (d, J=6.7 Hz, 3H); HRMS (ESI) calcd for C21H23N7O Cl2 [M+H]+ 460.1414, found 460.1415.

### N-[(2S)-1-(azetidin-1-yl)-3-methylbutan-2-yl]-3-{2-[(3,5-dichlorophenyl)amino]pyrimidin-4-yl}-1-methyl-1H-pyrazole-5-carboxamide (cpd 61) [(I), R1=3,5-dichlorophenyl, R2=methyl, R3=(2S)-1-(azetidin-1-yl)-3-methylbutan-2-yl, R4=H]

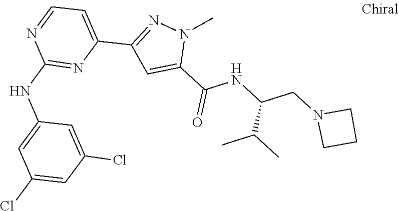 [SMILES: CC(C)[C@@H](CN1CCC1)NC(=O)c1cc(-c2ccnc(Nc3cc(Cl)cc(Cl)c3)n2)nn1C]

¹H NMR (500 MHz, DMSO-d6) δ ppm 10.10 (s, 1H), 8.60 (d, J=5.2 Hz, 1H), 8.32 (d, J=9.2 Hz, 1H), 7.99 (d, J=2.0 Hz, 2H), 7.45 (s, 1H), 7.40 (d, J=5.2 Hz, 1H), 7.13 (t, J=1.9 Hz, 1H), 4.12 (s, 3H), 3.83-3.68 (m, 1H), 3.22-2.95 (m, 4H), 2.47 (d, J=6.1 Hz, 2H), 1.92 (quin, J=6.9 Hz, 2H), 1.80 (dspt, J=6.4 Hz, 1H), 0.87 (t, J=7.3 Hz, 6H); HRMS (ESI) calcd for C23H27N7O Cl2 [M+H]+ 488.1727, found 488.174.

### N-[(2S)-1-(azetidin-1-yl)propan-2-yl]-1-methyl-3-(2-{[3-(trifluoromethyl)phenyl]amino} pyrimidin-4-yl)-1H-pyrazole-5-carboxamide (cpd 62) [(I), R1=3-(trifluoromethyl)phenyl, R2=methyl, R3=(2S)-1-(azetidin-1-yl)propan-2-yl, R4=H]

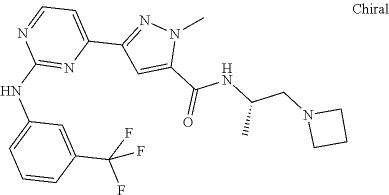 [SMILES: C[C@@H](CN1CCC1)NC(=O)c1cc(-c2ccnc(Nc3cccc(C(F)(F)F)c3)n2)nn1C]

¹H NMR (500 MHz, DMSO-d6) δ ppm 10.03 (s, 1H), 8.57 (d, J=5.2 Hz, 1H), 8.41 (d, J=8.4 Hz, 1H), 8.36 (t, J=1.9 Hz, 1H), 8.08 (dd, J=1.3, 8.2 Hz, 1H), 7.54 (t, J=8.0 Hz, 1H), 7.43 (s, 1H), 7.35 (d, J=5.2 Hz, 1H), 7.30 (d, J=7.6 Hz, 1H), 4.13 (s, 3H), 3.97-3.85 (m, 1H), 3.20-3.07 (m, 4H), 2.48-2.44 (m, 1H), 2.41-2.37 (m, 1H), 2.02-1.88 (m, 2H), 1.11 (d, J=6.7 Hz, 3H); HRMS (ESI) calcd for C22H24N7O F3 [M+H]+ 460.2067, found 460.2072.

### N-[(2S)-1-(azetidin-1-yl)propan-2-yl]-3-(2-{[3,5-bis(trifluoromethyl)phenyl]amino}pyrimidin-4-yl)-1-methyl-1H-pyrazole-5-carboxamide (cpd 63) [(I), R1=3,5-bis(trifluoromethyl)phenyl, R2=methyl, R3=(2S)-1-(azetidin-1-yl)propan-2-yl, R4=H]

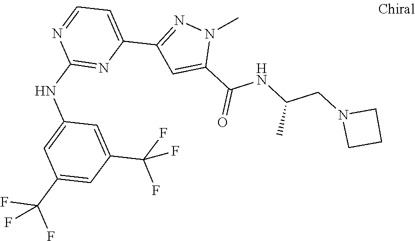 [SMILES: C[C@@H](CN1CCC1)NC(=O)c1cc(-c2ccnc(Nc3cc(C(F)(F)F)cc(C(F)(F)F)c3)n2)nn1C]

¹H NMR (500 MHz, DMSO-d6) δ ppm 10.40 (s, 1H), 8.64 (d, J=5.0 Hz, 1H), 8.60 (s, 2H), 8.39 (d, J=8.4 Hz, 1H), 7.61 (s, 1H), 7.42 (d, J=5.2 Hz, 1H), 7.40 (s, 1H), 4.13 (s, 3H), 3.97-3.85 (m, 1H), 3.22-3.05 (m, 4H), 2.47-2.42 (m, 1H), 2.42-2.37 (m, 1H), 2.01-1.87 (m, 2H), 1.10 (d, J=6.7 Hz, 3H); HRMS (ESI) calcd for C23H23N7O F6 [M+H]+ 528.1941, found 528.1951.

### N-[(2S)-1-(azetidin-1-yl)propan-2-yl]-3-{2-[(3,5-dimethoxyphenyl)amino]pyrimidin-4-yl}-1-methyl-1H-pyrazole-5-carboxamide (cpd 64) [(I), R1=3,5-dimethoxyphenyl, R2=methyl, R3=(2S)-1-(azetidin-1-yl)propan-2-yl, R4=H]

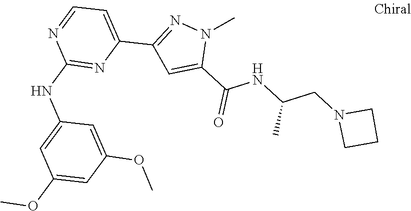 [SMILES: COc1cc(Nc2nccc(-c3cc(C(=O)N[C@@H](C)CN4CCC4)n(C)n3)n2)cc(OC)c1]

¹H NMR (500 MHz, DMSO-d6) δ ppm 9.59 (s, 1H), 8.52 (d, J=5.0 Hz, 1H), 8.42 (d, J=8.4 Hz, 1H), 7.46 (s, 1H), 7.28 (d, J=5.0 Hz, 1H), 7.17 (d, J=2.1 Hz, 2H), 6.13 (t, J=2.2 Hz, 1H), 4.13 (s, 3H), 3.96-3.85 (m, 1H), 3.75 (s, 6H), 3.20-3.05 (m, 4H), 2.48-2.32 (m, 2H), 1.95 (t, J=6.9 Hz, 2H), 1.10 (d, J=6.7 Hz, 3H); HRMS (ESI) calcd for C23H29N7O3 [M+H]+ 452.2405, found 452.2408.

### N-[(2S)-1-(dimethylamino)propan-2-yl]-3-{2-[(3-fluoro-5-methoxyphenyl)amino]pyrimidin-4-yl}-1-methyl-1H-pyrazole-5-carboxamide (cpd 65) [(I), R1=3-fluoro-5-methoxyphenyl, R2=methyl, R3=(2S)-1-(dimethylamino)propan-2-yl, R4=H]

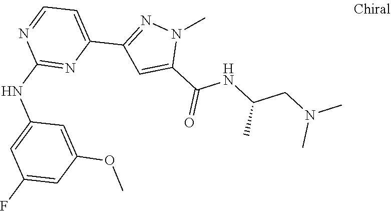 [SMILES: COc1cc(F)cc(Nc2nccc(-c3cc(C(=O)N[C@@H](C)CN(C)C)n(C)n3)n2)c1]

¹H NMR (500 MHz, DMSO-d6) δ ppm 9.84 (s, 1H), 8.56 (d, J=5.19 Hz, 1H), 8.49 (br. s., 1H), 7.46 (s, 1H), 7.40 (s, 1H), 7.32-7.37 (m, 2H), 6.41 (dt, J=10.87, 2.27 Hz, 1H), 4.14 (m, 4H), 3.78 (s, 3H), 2.54-2.05 (m. br., 8H) 1.14 (d, J=6.41 Hz, 3H); HRMS (ESI) calcd for C21H26N7O2F [M+H]+ 428.2205, found 428.2202.

### N-[(2S)-1-(azetidin-1-yl)propan-2-yl]-3-[2-(1,3-benzodioxol-5-ylamino)pyrimidin-4-yl]-1-methyl-1H-pyrazole-5-carboxamide (cpd 66) [(I), R1=1,3-benzodioxol-5-yl, R2=methyl, R3=(2S)-1-(azetidin-1-yl)propan-2-yl, R4=H]

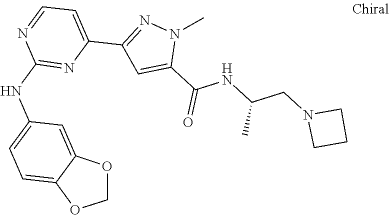 [SMILES: C[C@@H](CN1CCC1)NC(=O)c1cc(-c2ccnc(Nc3ccc4c(c3)OCO4)n2)nn1C]

¹H NMR (500 MHz, DMSO-d6) δ ppm 9.49 (s, 1H), 8.46 (d, J=5.0 Hz, 1H), 8.43 (d, J=8.2 Hz, 1H), 7.48 (d, J=2.0 Hz, 1H), 7.41 (s, 1H), 7.25 (dd, J=2.1, 8.5 Hz, 1H), 7.24 (d, J=5.0 Hz, 0H), 6.85 (d, J=8.5 Hz, 1H), 5.97 (s, 2H), 4.13 (s, 3H), 3.97-3.84 (m, 1H), 3.21-3.04 (m, 4H), 2.47 (dd, J=7.0, 11.6 Hz, 1H), 2.38 (dd, J=6.1, 11.6 Hz, 1H), 1.95 (quin, J=6.9 Hz, 2H), 1.10 (d, J=6.7 Hz, 3H); HRMS (ESI) calcd for C22H25N7O3 [M+H]+436.2092, found 436.21.

### 3-{2-[(3,5-dimethoxyphenyl)amino]pyrimidin-4-yl}-N-[(2S)-1-(dimethylamino)propan-2-yl]-1-methyl-1H-pyrazole-5-carboxamide (cpd 67) [(I), R1=3,5-dimethoxyphenyl, R2=methyl, R3=(2S)-1-(dimethylamino)propan-2-yl, R4=H]

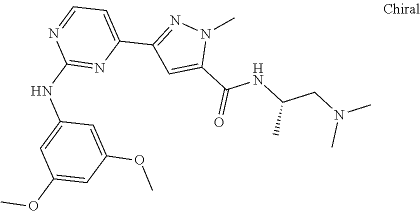

¹H NMR (500 MHz, DMSO-d6) δ ppm 9.60 (s, 1H), 8.52 (d, J=5.2 Hz, 1H), 8.46 (d, J=8.4 Hz, 1H), 7.46 (s, 1H), 7.28 (d, J=5.0 Hz, 1H), 7.17 (d, J=2.3 Hz, 2H), 6.13 (t, J=2.2 Hz, 1H), 4.13 (s, 3H), 4.17-4.07 (m, 1H), 3.75 (s, 6H), 2.43-2.31 (m, 1H), 2.18 (br. s., 7H), 1.12 (d, J=6.7 Hz, 3H); HRMS (ESI) calcd for C22H29N7O3 [M+H]+ 440.2405, found 440.2404.

### N-[(2S)-1-(azetidin-1-yl)propan-2-yl]-1-methyl-3-{2-[(3,4,5-trimethoxyphenyl)amino]pyrimidin-4-yl}-1H-pyrazole-5-carboxamide (cpd 68) [(I), R1=3,4,5-trimethoxyphenyl, R2=methyl, R3=(2S)-1-(azetidin-1-yl)propan-2-yl, R4=H]

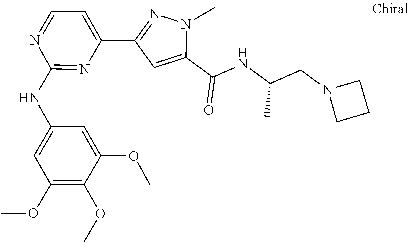 [SMILES: COc1cc(Nc2nccc(-c3cc(C(=O)N[C@@H](C)CN4CCC4)n(C)n3)n2)cc(OC)c1OC]

¹H NMR (500 MHz, DMSO-d6) δ ppm 9.52 (s, 1H), 8.50 (d, J=5.0 Hz, 1H), 8.40 (d, J=8.2 Hz, 1H), 7.49 (s, 1H), 7.31 (s, 2H), 7.26 (d, J=5.2 Hz, 1H), 4.13 (s, 3H), 3.94-3.86 (m, 1H), 3.80 (s, 6H), 3.63 (s, 3H), 3.13 (dd, J=6.8, 16.2 Hz, 4H), 2.45-2.31 (m, 2H), 2.04-1.87 (m, 2H), 1.10 (d, J=6.7 Hz, 3H); HRMS (ESI) calcd for C24H31N7O4 [M+H]+ 482.2511, found 482.2514.

### 3-{2-[(3,5-dimethoxyphenyl)amino]pyrimidin-4-yl}-1-methyl-N-[(2S)-1-(pyrrolidin-1-yl)propan-2-yl]-1H-pyrazole-5-carboxamide (cpd 69) [(I), R1=3,5-dimethoxyphenyl, R2=methyl, R3=(2S)-1-pyrrolidin-1-ylpropan-2-yl, R4=H]

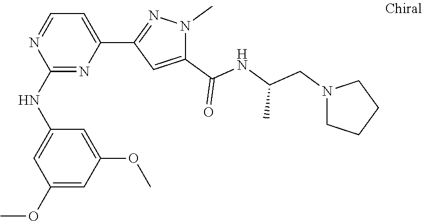 [SMILES: COc1cc(Nc2nccc(-c3cc(C(=O)N[C@@H](C)CN4CCCC4)n(C)n3)n2)cc(OC)c1]

¹H NMR (500 MHz, DMSO-d6) δ ppm 9.60 (s, 1H), 8.52 (d, J=5.0 Hz, 1H), 8.48 (d, J=8.4 Hz, 1H), 7.46 (s, 1H), 7.28 (d, J=5.0 Hz, 1H), 7.18 (d, J=2.3 Hz, 2H), 6.13 (t, J=2.3 Hz, 1H), 4.12 (s, 3H), 4.12-4.06 (m, 1H), 3.75 (s, 6H), 2.49-2.39 (m, 5H), 1.67 (t, J=2.9 Hz, 4H), 1.14 (d, J=6.6 Hz, 3H); HRMS (ESI) calcd for C24H31N7O3 [M+H]+ 466.2561, found 466.256.

### 3-{2-[(3-methoxyphenyl)amino]pyrimidin-4-yl}-1-methyl-N-[(2S)-1-(pyrrolidin-1-yl)propan-2-yl]-1H-pyrazole-5-carboxamide (cpd 70) [(I), R1=3-methoxyphenyl, R2=methyl, R3=(2S)-1-pyrrolidin-1-ylpropan-2-yl, R4=H]

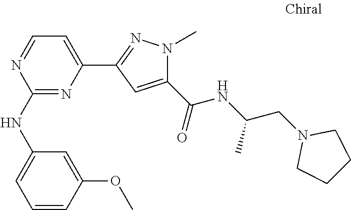 [SMILES: COc1cccc(Nc2nccc(-c3cc(C(=O)N[C@@H](C)CN4CCCC4)n(C)n3)n2)c1]

¹H NMR (500 MHz, DMSO-d6) δ ppm 9.63 (s, 1H), 8.55-8.46 (m, 2H), 7.65-7.61 (m, 2H), 7.37 (dd, J=1.2, 8.1 Hz, 1H), 7.29 (d, J=5.2 Hz, 1H), 7.19 (t, J=8.2 Hz, 1H), 6.54 (dd, J=1.8, 8.1 Hz, 1H), 4.13 (s, 4H), 3.77 (s, 3H), 2.62-2.51 (m, 6H), 1.69 (br. s., 4H), 1.16 (d, J=6.7 Hz, 3H); HRMS (ESI) calcd for C23H29N7O2 [M+H]+436.2456, found 436.2455.

### 3-{2-[(3,5-dimethoxyphenyl)amino]pyrimidin-4-yl}-N-[(2S)-1-(dimethylamino)-1-oxopropan-2-yl]-1-methyl-1H-pyrazole-5-carboxamide (cpd 71) [(I), R1=3,5-dimethoxyphenyl, R2=methyl, R3=(2S)-1-(dimethylamino)-1-oxopropan-2-yl, R4=H]

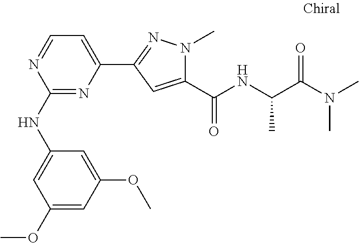

¹H NMR (500 MHz, DMSO-d6) δ ppm 9.62 (s, 1H), 8.87 (d, J=7.5 Hz, 1H), 8.52 (d, J=5.0 Hz, 1H), 7.59 (s, 1H), 7.28 (d, J=5.0 Hz, 1H), 7.18 (d, J=2.1 Hz, 2H), 6.12 (t, J=2.2 Hz, 1H), 4.88 (t, J=7.2 Hz, 1H), 4.12 (s, 3H), 3.75 (s, 6H), 3.06 (s, 3H), 2.86 (s, 3H), 1.32-1.26 (m, 3H); HRMS (ESI) calcd for C22H27N7O4 [M+H]+454.2198, found 454.2198.

### 3-{2-[(3,5-dimethoxyphenyl)amino]pyrimidin-4-yl}-N-[2-(dimethylamino)ethyl]-1-methyl-1H-pyrazole-5-carboxamide (cpd 72) [(I), R1=3,5-dimethoxyphenyl, R2=methyl, R3=2-(dimethylamino)ethyl, R4=H]

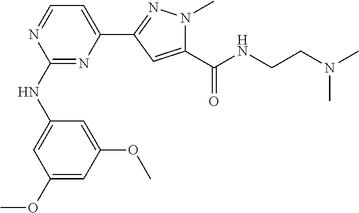

¹H NMR (500 MHz, DMSO-d6) δ ppm 9.60 (s, 1H), 8.64 (t, J=5.7 Hz, 1H), 8.52 (d, J=5.0 Hz, 1H), 7.42 (s, 1H), 7.28 (d, J=5.0 Hz, 1H), 7.17 (d, J=2.3 Hz, 2H), 6.13 (t, J=2.2 Hz, 1H), 4.13 (s, 3H), 3.75 (s, 6H), 2.40 (t, J=6.7 Hz, 2H), 2.19 (s, 6H); HRMS (ESI) calcd for C21H27N7O3 [M+H]+ 426.2248, found 426.224.

### N-[(2S)-1-(dimethylamino)propan-2-yl]-3-{2-[(1,5-dimethyl-1H-pyrazol-3-yl)amino]pyrimidin-4-yl}-1-methyl-1H-pyrazole-5-carboxamide (cpd 73) [(I), R1=1,5-dimethylpyrazol-3-yl, R2=methyl, R3=(2S)-1-(dimethylamino)propan-2-yl, R4=H]

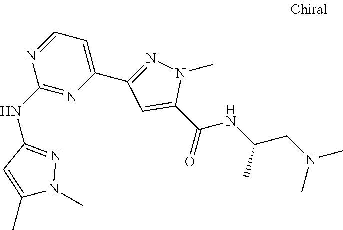 [SMILES: Cc1cc(Nc2nccc(-c3cc(C(=O)N[C@@H](C)CN(C)C)n(C)n3)n2)nn1C]

¹H NMR (500 MHz, DMSO-d6) δ ppm 9.52 (s, 1H), 8.49 (d, J=8.2 Hz, 1H), 8.42 (d, J=5.2 Hz, 1H), 7.44 (s, 1H), 7.21 (d, J=5.0 Hz, 1H), 6.56 (s, 1H), 4.17-4.04 (m, 4H), 3.63 (s, 3H), 2.40 (dd, J=7.9, 12.0 Hz, 1H), 2.26 (s, 3H), 2.22-2.19 (m, 1H), 2.17 (s, 6H), 1.16-1.11 (m, 3H); HRMS (ESI) calcd for C19H27N9O [M+H]+ 398.2412, found 398.2413.

### N-[(2S)-1-(azetidin-1-yl)propan-2-yl]-3-{2-[(1,5-dimethyl-1H-pyrazol-3-yl)amino]pyrimidin-4-yl}-1-methyl-1H-pyrazole-5-carboxamide (cpd 74) [(I), R1=1,5-dimethylpyrazol-3-yl, R2=methyl, R3=(2S)-1-(azetidin-1-yl)propan-2-yl, R4=H]

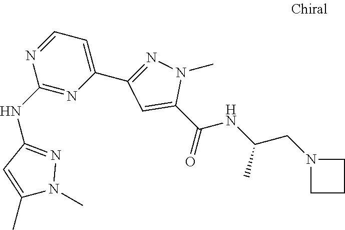 [SMILES: Cc1cc(Nc2nccc(-c3cc(C(=O)N[C@@H](C)CN4CCC4)n(C)n3)n2)nn1C]

¹H NMR (500 MHz, DMSO-d6) δ ppm 9.53 (s, 1H), 8.47 (d, J=8.2 Hz, 1H), 8.42 (d, J=5.0 Hz, 1H), 7.44 (s, 1H), 7.21 (d, J=5.0 Hz, 1H), 6.57 (s, 1H), 4.13 (s, 3H), 3.97-3.87 (m, 1H), 3.63 (s, 3H), 3.29-3.02 (m, 4H), 2.47 (d, J=7.0 Hz, 0H), 2.42-2.35 (m, 1H), 2.27 (s, 3H), 1.94 (quin, J=6.9 Hz, 2H), 1.11 (d, J=6.7 Hz, 3H); HRMS (ESI) calcd for C20H27N9O [M+H]+ 410.2412, found 410.2407.

### N-[(2S)-1-(dimethylamino)propan-2-yl]-1-methyl-3-{2-[(1-methyl-1H-pyrazol-4-yl)amino]pyrimidin-4-yl}-1H-pyrazole-5-carboxamide (cpd 75) [(I), R1=1-methylpyrazol-4-yl, R2=methyl, R3=(2S)-1-(dimethylamino)propan-2-yl, R4=H]

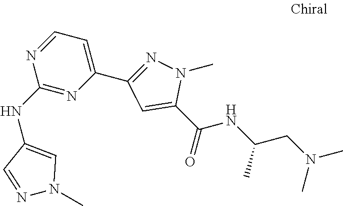 [SMILES: C[C@@H](CN(C)C)NC(=O)c1cc(-c2ccnc(Nc3cnn(C)c3)n2)nn1C]

¹H NMR (500 MHz, DMSO-d6) δ ppm 9.49 (br. s., 1H), 8.46 (d, J=8.4 Hz, 1H), 8.43 (d, J=5.0 Hz, 1H), 7.97 (br. s., 1H), 7.61-7.40 (m, 2H), 7.17 (d, J=5.0 Hz, 1H), 4.14 (s, 3H), 4.11 (s, 0H), 3.84 (s, 3H), 2.40 (dd, J=7.9, 12.2 Hz, 1H), 2.24-2.19 (m, 1H), 2.17 (s, 6H), 1.14 (d, J=6.7 Hz, 3H); HRMS (ESI) calcd for C20H27N9O [M+H]+ 410.2412, found 410.2407.

### N-[(2S)-1-(azetidin-1-yl)propan-2-yl]-1-methyl-3-[2-(tetrahydro-2H-pyran-4-ylamino)pyrimidin-4-yL]-1H-pyrazole-5-carboxamide (cpd 76) [(I), R1=oxan-4-yl, R2=methyl, R3=(2S)-1-(azetidin-1-yl)propan-2-yl, R4=H]

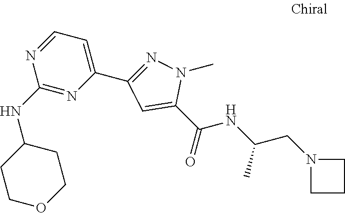 [SMILES: C[C@@H](CN1CCC1)NC(=O)c1cc(-c2ccnc(NC3CCOCC3)n2)nn1C]

¹H NMR (500 MHz, DMSO-d6) δ ppm 8.36 (d, J=8.2 Hz, 1H), 8.30 (d, J=4.9 Hz, 1H), 7.37 (s, 1H), 7.09 (br. s., 1H), 7.03 (d, J=5.0 Hz, 1H), 4.11 (s, 3H), 4.06-3.95 (m, 1H), 3.94-3.83 (m, 3H), 3.40 (t, J=11.4 Hz, 2H), 3.12 (qd, J=6.5, 19.5 Hz, 4H), 2.48-2.44 (m, 1H), 2.39-2.33 (m, 1H), 1.94 (quin, J=6.9 Hz, 2H), 1.84 (d, J=12.4 Hz, 2H), 1.59-1.47 (m, 2H), 1.10 (d, J=6.7 Hz, 3H); HRMS (ESI) calcd for C20H29N7O2 [M+H]+ 400.2456, found 400.2455.

### N,N,1-trimethyl-3-[2-(tetrahydro-2H-pyran-4-ylamino)pyrimidin-4-yl]-1H-pyrazole-5-carboxamide (cpd 77) [(I), R1=oxan-4-yl, R2=methyl, R3=methyl, R4=methyl]

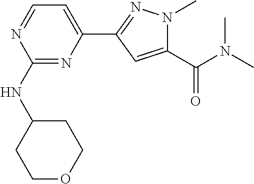 [SMILES: CN(C)C(=O)c1cc(-c2ccnc(NC3CCOCC3)n2)nn1C]

¹H NMR (500 MHz, DMSO-d6) δ ppm 8.30 (d, J=4.9 Hz, 1H), 7.15 (d, J=7.6 Hz, 1H), 7.03 (d, J=5.2 Hz, 1H), 6.96 (br. s., 1H), 3.98 (br. s., 1H), 3.91 (s, 3H), 3.89-3.84 (m, 2H), 3.43-3.36 (m, 1H), 3.04 (d, J=19.7 Hz, 6H), 1.83 (d, J=12.0 Hz, 2H), 1.56-1.44 (m, 2H); HRMS (ESI) calcd for C16H22N6O2 [M+H]+ 331.1877, found 331.187.

### 3-{2-[(3-cyano-5-methoxyphenyl)amino]pyrimidin-4-yl}-N-[(2S)-1-(dimethylamino)propan-2-yl]-1-methyl-1H-pyrazole-5-carboxamide (cpd 78) [(I), R1=3-cyano-5-methoxyphenyl, R2=methyl, R3=(2S)-1-(dimethylamino)propan-2-yl, R4=H]

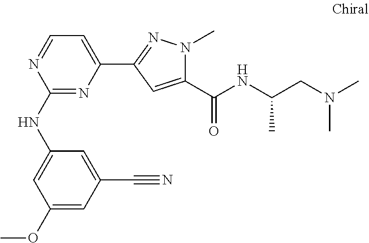

¹H NMR (500 MHz, DMSO-d6) δ ppm 10.01 (s, 1H), 8.59 (d, J=5.2 Hz, 1H), 8.44 (d, J=8.4 Hz, 1H), 7.91 (t, J=2.1 Hz, 1H), 7.83 (t, J=1.5 Hz, 1H), 7.46 (s, 1H), 7.36 (d, J=5.2 Hz, 1H), 7.01 (dd, J=1.3, 2.4 Hz, 1H), 4.13 (s, 3H), 4.10 (s, 0H), 3.83 (s, 3H), 2.41-2.34 (m, 1H), 2.22-2.18 (m, 1H), 2.17 (s, 5H), 1.13 (d, J=6.6 Hz, 3H); HRMS (ESI) calcd for C22H26N8O2 [M+H]+ 435.2252, found 435.2252.

### N-[(2S)-1-(azetidin-1-yl)propan-2-yl]-3-{2-[(3-cyano-5-methoxyphenyl)amino]pyrimidin-4-yl}-1-methyl-1H-pyrazole-5-carboxamide (cpd 79) [(I), R1=3-cyano-5-methoxyphenyl, R2=methyl, R3=(2S)-1-(azetidin-1-yl)propan-2-yl, R4=H]

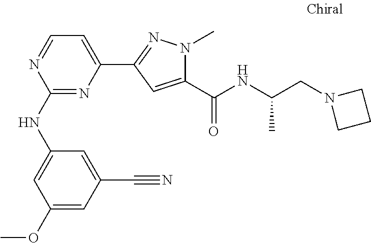 [SMILES: COc1cc(C#N)cc(Nc2nccc(-c3cc(C(=O)N[C@@H](C)CN4CCC4)n(C)n3)n2)c1]

¹H NMR (500 MHz, DMSO-d6) δ ppm 10.01 (s, 1H), 8.59 (d, J=5.2 Hz, 1H), 8.41 (d, J=8.4 Hz, 1H), 7.91 (t, J=2.1 Hz, 1H), 7.83 (t, J=1.5 Hz, 1H), 7.46 (s, 1H), 7.36 (d, J=5.0 Hz, 1H), 7.01 (dd, J=1.4, 2.3 Hz, 1H), 4.13 (s, 3H), 3.96-3.87 (m, 1H), 3.83 (s, 3H), 3.19-3.06 (m, 4H), 2.46 (dd, J=7.0, 11.6 Hz, 1H), 2.40-2.33 (m, 1H), 1.94 (quin, J=7.0 Hz, 2H), 1.10 (d, J=6.7 Hz, 3H); HRMS (ESI) calcd for C23H26N8O2 [M+H]+ 447.2252, found 447.2251.

### N-[(2S)-1-(azetidin-1-yl)propan-2-yl]-3-{2-[(5-methoxypyridin-3-yl)amino]pyrimidin-4-yl}-1-methyl-1H-pyrazole-5-carboxamide (cpd 80) [(I), R1=5-methoxypyridin-3-yl, R2=methyl, R3=(2S)-1-(azetidin-1-yl)propan-2-yl, R4=H]

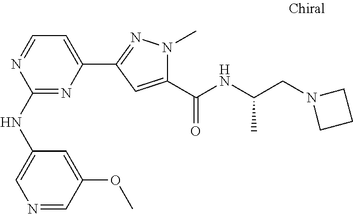 [SMILES: COc1cncc(Nc2nccc(-c3cc(C(=O)N[C@@H](C)CN4CCC4)n(C)n3)n2)c1]

¹H NMR (500 MHz, DMSO-d6) δ ppm 9.89 (s, 1H), 8.56 (d, J=5.2 Hz, 1H), 8.52 (d, J=2.0 Hz, 1H), 8.44 (d, J=8.4 Hz, 1H), 8.12 (t, J=2.3 Hz, 1H), 7.91 (d, J=2.6 Hz, 1H), 7.47 (s, 1H), 7.34 (d, J=5.0 Hz, 1H), 4.14 (s, 3H), 3.95-3.88 (m, 1H), 3.86 (s, 3H), 3.19-3.06 (m, 4H), 2.46 (dd, J=7.0, 11.7 Hz, 1H), 2.40-2.32 (m, 1H), 1.94 (quin, J=6.9 Hz, 2H), 1.10 (d, J=6.7 Hz, 3H); HRMS (ESI) calcd for C21H26N8O2 [M+H]+ 423.2252, found 423.2252.

### 3-{2-[(3,5-dimethylphenyl)amino]pyrimidin-4-yl}-1-methyl-N-[(3S)-piperidin-3-yl]-1H-pyrazole-5-carboxamide hydrochloride (cpd 81) [(I), R1=3,5-dimethylphenyl, R2=methyl, R3=(3S)-piperidin-3-yl, R4=H]

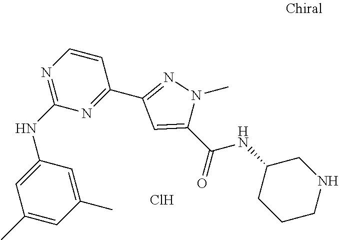 [SMILES: Cc1cc(C)cc(Nc2nccc(-c3cc(C(=O)N[C@H]4CCCNC4)n(C)n3)n2)c1 ; Cl]

¹H NMR (500 MHz, DMSO-d6) δ ppm 9.49 (s, 1H), 8.99-8.90 (br. s. 1H, 8.85 (d, J=7.8 Hz, 1H), 8.87-8.80 (br. s., 1H), 8.50 (d, J=5.03 Hz, 1H), 7.52 (s, 1H), 7.47 (s, 2H), 7.26 (d, J=5.2 Hz, 1H), 6.62 (s, 1H), 4.14 (s, 3H), 3.33 (m partially overlapped by water signal, 2H), 3.22-3.14 (m, 1H), 2.92-2.77 (m, 2H), 2.27 (s, 6H), 1.95-1.50 (m, 4H); HRMS (ESI) calcd for C22H28N7OCl [M+H]+ 406.235, found 406.2349.

### N-(2-aminocyclohexyl)-3-{2-[(3,5-dimethylphenyl)amino]pyrimidin-4-yl}-1-methyl-1H-pyrazole-5-carboxamide hydrochloride (cpd 82) [(I), R1=3,5-dimethylphenyl, R2=methyl, R3=2-aminocyclohexyl, R4=H]

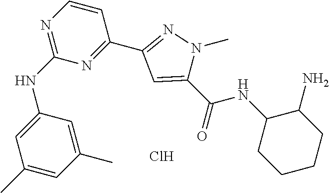 [SMILES: Cc1cc(C)cc(Nc2nccc(-c3cc(C(=O)NC4CCCCC4N)n(C)n3)n2)c1 ; Cl]

¹H NMR (500 MHz, DMSO-d6) δ ppm (s, 1H), 8.79 (d, J=8.7 Hz, 1H), 8.50 (d, J=5.2 Hz, 1H), 7.85 (br. s., 3H), 7.51 (s, 1H), 7.48 (s, 2H), 7.26 (d, J=5.0 Hz, 1H), 6.62 (s, 1H), 4.15 (s, 3H), 3.88-3.80 (m, 1H), 3.00-2.90 (m, 1H), 2.27 (s, 6H), 2.10-1.20 (m, 8H); HRMS (ESI) calcd for C23H30N7OCl [M+H]+ 420.2507, found 420.2503.

### 3-{2-[(3,5-dimethylphenyl)amino]pyrimidin-4-yl}-1-methyl-N-[2-(pyrrolidin-1-yl)ethyl]-1H-pyrazole-5-carboxamide (cpd 83) [(I), R1=3,5-dimethylphenyl, R2=methyl, R3=2-pyrrolidin-1-ylethyl, R4=H]

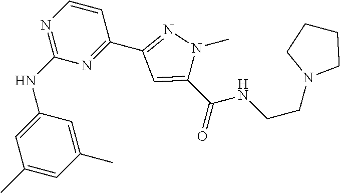 [SMILES: Cc1cc(C)cc(Nc2nccc(-c3cc(C(=O)NCCN4CCCC4)n(C)n3)n2)c1]

¹H NMR (500 MHz, DMSO-d6) δ ppm 9.47 (s, 1H), 8.86 (br. s., 1H), 8.50 (d, J=5.0 Hz, 1H), 7.48-7.42 (m, 3H), 7.26 (d, J=5.2 Hz, 1H), 6.62 (s, 1H), 4.15 (s, 3H), 3.44 (br. s., 2H), 2.70 (br. s. 2H), 1.79 (br. s. 4H); HRMS (ESI) calcd for C23H29N7O [M+H]+ 420.2507, found 420.2491.

### 3-{2-[(3,5-dimethylphenyl)amino]pyrimidin-4-yl}-1-methyl-N-[2-(piperidin-1-yl)ethyl]-1H-pyrazole-5-carboxamide (cpd 84) [(I), R1=3,5-dimethylphenyl, R2=methyl, R3=2-piperidin-1-ylethyl, R4=H]

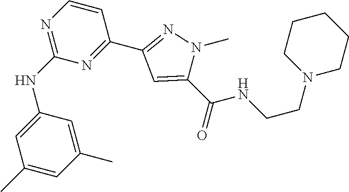 [SMILES: Cc1cc(C)cc(Nc2nccc(-c3cc(C(=O)NCCN4CCCCC4)n(C)n3)n2)c1]

¹H NMR (500 MHz, DMSO-d6) δ ppm 9.46 (s, 1H), 8.72 (br. s., 1H), 8.49 (d, J=5.0 Hz, 1H), 7.46 (s, 2H), 7.41 (s, 1H), 7.26 (d, J=5.0 Hz, 1H), 6.62 (s, 1H), 4.14 (s, 3H), 3.34 (br. s. partially overlapped by water signal, 2H), 2.41 (br. s., 6H), 2.27 (s, 6H), 1.67-1.30 (m, 6H); HRMS (ESI) calcd for C24H31N7O [M+H]+ 434.2663, found 434.2661.

### (3-{2-[(3,5-dimethylphenyl)amino]pyrimidin-4-yl}-1-methyl-1H-pyrazol-5-yl)[(3R)-3-hydroxypyrrolidin-1-yl]methanone (cpd 85) [(I), R1=3,5-dimethylphenyl, R2=methyl, NR3R4=(3R)-3-hydroxypyrrolidin-1-ylamino]

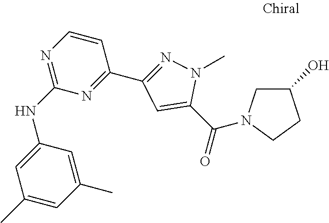 [SMILES: Cc1cc(C)cc(Nc2nccc(-c3cc(C(=O)N4CC[C@@H](O)C4)n(C)n3)n2)c1]

¹H NMR (500 MHz, DMSO-d6) (mix of conformers) δ ppm 9.53 and 9.52 (2×s, 1H), 8.50 (d, J=5.0 Hz, 1H), 7.48 (d, J=2.7 Hz, 2H), 7.27 (d, J=5.0 Hz, 1H), 7.22 and 7.16 (2×s, 1H), 6.60 (s, 1H), 5.07 (t, J=3.9 Hz, 1H), 4.35 and 4.31 (2×m, 1H), 4.05 (s, 3H), 3.81-3.40 (m, 4H), 2.25 (s, 6H), 2.03-1.80 (m, 2H); HRMS (ESI) calcd for C21H24N6O2 [M+H]+ 393.2034, found 393.203.

### [(3S)-3-(dimethylamino)pyrrolidin-1-yl](3-{2-[(3,5-dimethylphenyl)amino]pyrimidin-4-yl}-1-methyl-1H-pyrazol-5-yl)methanone (cpd 86) [(I), R1=3,5-dimethylphenyl, R2=methyl, NR3R4=(3S)-3-(dimethylamino)pyrrolidin-1-ylamino]

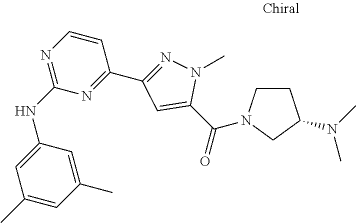

¹H NMR (500 MHz, DMSO-d6) (mix of conformers) δ ppm 9.52 (s, 1H), 8.50 (d, J=5.2 Hz, 1H), 7.48 (s, 2H), 7.28 and 7.26 (2×d J=5.2 Hz, 1H), 7.18 and 7.17 (2×s, 1H), 6.60 (s, 1H), 4.04 and 4.03 (2×s, 3H), 3.86-3.21 (m, 4H), 2.80-2.70 (m, 1H), 2.25 (s, 6H), 2.19 (s, 3H), 2.14 (s, 3H), 2.12-2.00 (m, 1H), 1.84-1.70 (m, 1H); HRMS (ESI) calcd for C23H29N7O [M+H]+ 420.2507, found 420.2506.

### [(3R)-3-(dimethylamino)pyrrolidin-1-yl](3-{2-[(3,5-dimethylphenyl)amino]pyrimidin-4-yl}-1-methyl-1H-pyrazol-5-yl)methanone (cpd 87) [(I), R1=3,5-dimethylphenyl, R2=methyl, NR3R4=(3R)-3-(dimethylamino)pyrrolidin-1-ylamino]

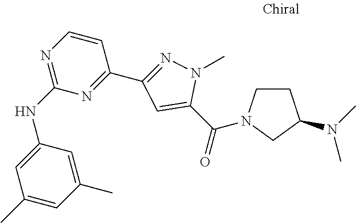

¹H NMR (500 MHz, DMSO-d6) (mix of conformers) δ ppm 9.52 (s, 1H), 8.50 (d, J=5.2 Hz, 1H), 7.48 (s, 2H), 7.28 and 7.26 (2×d J=5.2 Hz, 1H), 7.18 and 7.17 (2×s, 1H), 6.60 (s, 1H), 4.04 and 4.03 (2×s, 3H), 3.86-3.21 (m, 4H), 2.80-2.70 (m, 1H), 2.25 (s, 6H), 2.19 (s, 3H), 2.14 (s, 3H), 2.12-2.00 (m, 1H), 1.84-1.70 (m, 1H); HRMS (ESI) calcd for C23H29N7O [M+H]+ 420.2507, found 420.2503.

### (3-aminopyrrolidin-1-yl)(3-{2-[(3,5-dimethylphenyl)amino]pyrimidin-4-yl}-1-methyl-1H-pyrazol-5-yl)methanone (cpd 88) [(I), R1=3,5-dimethylphenyl, R2=methyl, NR3R4=3-aminopyrrolidin-1-ylamino]

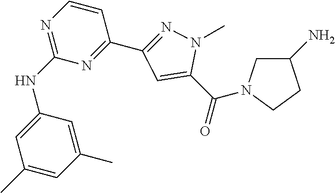 [SMILES: Cc1cc(C)cc(Nc2nccc(-c3cc(C(=O)N4CCC(N)C4)n(C)n3)n2)c1]

¹H NMR (500 MHz, DMSO-d6) (mix of conformers) δ ppm 9.54 and 9.52 (2×s, 1H), 8.50 (d, J=5.0 Hz, 1H), 7.48 (s, 2H), 7.27 and 7.26 (2×d, J=5.0 Hz, 1H), 7.22 and 7.15 (2×s, 1H), 6.60 (s, 1H), 4.05 (s, 3H), 3.84-3.22 (m, 5H), 2.25 (s, 6H), 2.04-1.95 (m, 1H), 1.73-1.64 (m, 1H); HRMS (ESI) calcd for C21H25N7O [M+H]+ 392.2194, found 392.2194.

### (3-{2-[(3,5-dimethylphenyl)amino]pyrimidin-4-yl}-1-methyl-1H-pyrazol-5-yl)[(2S)-2-(hydroxymethyl)pyrrolidin-1-yl]methanone (cpd 89) [(I), R1=3,5-dimethylphenyl, R2=methyl, NR3R4=(2S)-2-(hydroxymethyl)pyrrolidin-1-ylamino]

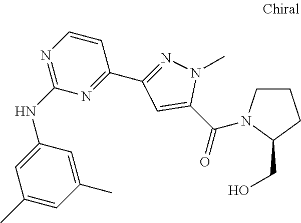 [SMILES: Cc1cc(C)cc(Nc2nccc(-c3cc(C(=O)N4CCC[C@H]4CO)n(C)n3)n2)c1]

¹H NMR (500 MHz, DMSO-d6) (mix of conformers) δ ppm 9.51 (s, 1H), 8.49 (d, J=5.0 Hz, 1H), 7.48 (s, 2H), 7.26 (d, J=5.0 Hz, 1H), 7.18 and 7.10 (2×s, 1H), 6.60 (s, 1H), 4.91 and 4.82 (2×t, J=5.8 Hz, 1H), 4.18 and 4.12 (2×m, 1H), 4.03 and 3.93 (2×s, 3H), 3.70-3.15 (m, 4H), 2.25 (s, 6H), 2.00-1.74 (m, 4H); HRMS (ESI) calcd for C22H26N6O2 [M+H]+ 407.219, found 407.2193.

### (3-{2-[(3,5-dimethylphenyl)amino]pyrimidin-4-yl}-1-methyl-1H-pyrazol-5-yl)[(3S)-3-hydroxypyrrolidin-1-yl]methanone (cpd 90) [(I), R1=3,5-dimethylphenyl, R2=methyl, NR3R4=(3S)-3-hydroxypyrrolidin-1-ylamino]

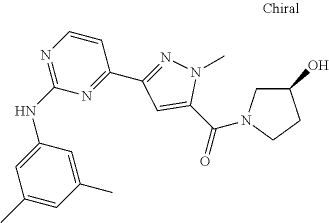 [SMILES: Cc1cc(C)cc(Nc2nccc(-c3cc(C(=O)N4CC[C@H](O)C4)n(C)n3)n2)c1]

¹H NMR (500 MHz, DMSO-d6) (mix of conformers) δ ppm 9.53 and 9.52 (2×s, 1H), 8.50 (d, J=5.0 Hz, 1H), 7.48 (s, 2H), 7.27 (d, J=5.0 Hz, 1H), 7.22 and 7.16 (2×s, 1H), 6.60 (s, 1H), 5.09 and 5.08 (2×d, J=4.6 Hz, 1H), 4.35 and 4.32 (2×m, 1H), 4.05 and 4.04 (2×s, 3H), 3.78-3.54 (m, 4H), 2.25 (s, 6H), 2.03-1.80 (m, 2H); HRMS (ESI) calcd for C21H24N6O2 [M+H]+ 393.2034, found 393.2027.

### 3-{2-[(3,5-dimethylphenyl)amino]pyrimidin-4-yl}-N-[(1R,2S)-2-hydroxycyclohexyl]-1-methyl-1H-pyrazole-5-carboxamide (cpd 91) [(I), R1=3,5-dimethylphenyl, R2=methyl, R3=(1R,2S)-2-hydroxycyclohexyl, R4=H]

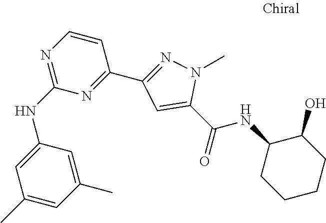 [SMILES: Cc1cc(C)cc(Nc2nccc(-c3cc(C(=O)N[C@@H]4CCCC[C@@H]4O)n(C)n3)n2)c1]

¹H NMR (500 MHz, DMSO-d6) δ ppm 9.47 (s, 1H), 8.49 (d, J=5.0 Hz, 1H), 8.15 (d, J=7.8 Hz, 1H), 7.49 (s, 1H), 7.48 (s, 2H), 7.25 (d, J=5.0 Hz, 1H), 6.61 (s, 1H), 4.65 (d, J=3.8 Hz, 1H), 4.12 (s, 3H), 3.85 (m, 1H), 3.83 (m, 1H), 2.26 (s, 6H), 1.77-1.25 (m, 8H); HRMS (ESI) calcd for C23H28N6O2 [M+H]+ 421.2347, found 421.2345.

### N-[(1S,2R)-2-aminocyclohexyl]-3-{2-[(3,5-dimethylphenyl)amino]pyrimidin-4-yl}-1-methyl-1H-pyrazole-5-carboxamide hydrochloride (cpd 92) [(I), R1=3,5-dimethylphenyl, R2=methyl, R3=(1S,2R)-2-aminocyclohexyl, R4=H]

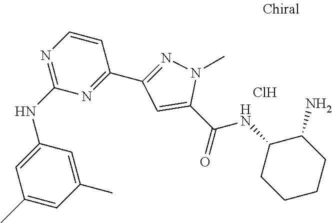 [SMILES: Cc1cc(C)cc(Nc2nccc(-c3cc(C(=O)N[C@H]4CCCC[C@H]4N)n(C)n3)n2)c1 ; Cl]

¹H NMR (500 MHz, DMSO-d6) δ ppm 9.51 (s, 1H), 8.50 (d, J=5.0 Hz, 1H), 8.47 (d, J=7.9 Hz, 1H), 7.87 (br. s., 3H), 7.60 (s, 1H), 7.51 (s, 2H), 7.27 (d, J=5.0 Hz, 1H), 6.62 (s, 1H), 4.33 (m, 1H), 4.14 (s, 3H), 3.37 (m overlapped by water signal, 1H), 2.28 (s, 6H), 1.80-1.34 (m, 8H); HRMS (ESI) calcd for C23H30N7OCl [M+H]+420.2507, found 420.2509.

### N-[(2S)-1-hydroxypropan-2-yl]-1-methyl-3-{2-[(1,2,3-trimethyl-1H-indol-5-yl)amino]pyrimidin-4-yl}-1H-pyrazole-5-carboxamide (cpd 93) [(I), R1=1,2,3-trimethylindol-5-yl, R2=methyl, R3=(2S)-1-hydroxypropan-2-yl, R4=H]

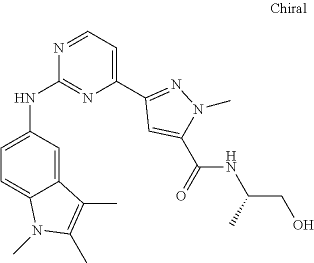 [SMILES: Cc1c(C)n(C)c2ccc(Nc3nccc(-c4cc(C(=O)N[C@@H](C)CO)n(C)n4)n3)cc12]

¹H NMR (500 MHz, DMSO-d6) δ ppm 9.32 (s, 1H), 8.44 (d, J=5.0 Hz, 1H), 8.43 (d, J=7.3 Hz, 1H), 8.10 (br. s., 1H), 7.46 (s, 1H), 7.29 (dd, J=8.7, 2.0 Hz, 1H), 7.25 (d, J=8.7 Hz, 1H), 7.18 (d, J=5.0 Hz, 1H), 4.75 (t, J=5.8 Hz, 1H), 4.13 (s, 3H), 3.99 (m, 1H), 3.62 (s, 3H), 3.47-3.30 (m partially overlapped by water signal, 2H), 2.32 (s, 3H), 2.20 (s, 3H), 1.13 (d, J=6.7 Hz, 3H); HRMS (ESI) calcd for C23H27N7O2 [M+H]+ 434.2299, found 434.2291.

### 3-{2-[(3,5-dimethylphenyl)amino]pyrimidin-4-yl}-1-methyl-N-[(2S)-1-(pyrrolidin-1-yl)propan-2-yl]-1H-pyrazole-5-carboxamide (cpd 94) [(I), R1=3,5-dimethylphenyl, R2=methyl, R3=(2S)-1-pyrrolidin-1-ylpropan-2-yl, R4=H]

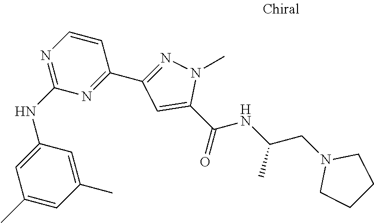 [SMILES: Cc1cc(C)cc(Nc2nccc(-c3cc(C(=O)N[C@@H](C)CN4CCCC4)n(C)n3)n2)c1]

¹H NMR (500 MHz, DMSO-d6) δ ppm 9.47 (s, 1H), 8.54 (d, J=8.1 Hz, 1H), 8.49 (d, J=5.0 Hz, 1H), 7.48 (s, 2H), 7.45 (s, 1H), 7.25 (d, J=5.0 Hz, 1H), 6.62 (s, 1H), 4.13 (s, 3H), 4.17-4.07 (m, 1H), 2.60-2.35 (m partially overlapped by DMSO signal, 6H), 2.27 (s, 6H), 1.72-1.62 (m, 4H), 1.14 (d, J=6.6 Hz, 3H); HRMS (ESI) calcd for C24H31N7O [M+H]+ 434.2663, found 434.2684.

### N-[(2S)-1-(azetidin-1-yl)propan-2-yl]-3-{2-[(3,5-dimethylphenyl)amino]pyrimidin-4-yl}-1-methyl-1H-pyrazole-5-carboxamide (cpd 95) [(I), R1=3,5-dimethylphenyl, R2=methyl, R3=(2S)-1-(azetidin-1-yl)propan-2-yl, R4=H]

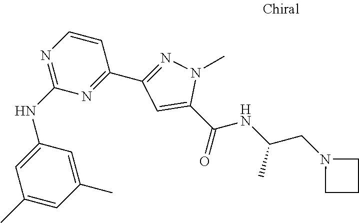 [SMILES: Cc1cc(C)cc(Nc2nccc(-c3cc(C(=O)N[C@@H](C)CN4CCC4)n(C)n3)n2)c1]

¹H NMR (500 MHz, DMSO-d6) δ ppm 9.50 (s, 1H), 8.55 (br. s., 1H), 8.52 (d, J=5.0 Hz, 1H), 7.51 (s, 2H), 7.48 (s, 1H), 7.29 (d, J=5.0 Hz, 1H), 6.65 (s, 1H), 4.17 (s, 3H), 4.05-3.94 (m, 1H), 3.50-3.10 (m partially overlapped by water signal, 6H), 2.30 (s, 6H), 2.10-1.93 (m, 2H), 1.14 (d, J=6.7 Hz, 3H); HRMS (ESI) calcd for C23H29N7O [M+H]+ 420.2507, found 420.252.

### (1S,4R)-2,5-diazabicyclo[2.2.1]hept-2-yl(3-{2-[(3,5-dimethylphenyl)amino]pyrimidin-4-yl}-1-methyl-1H-pyrazol-5-yl)methanone hydrochloride (cpd 96) [(I), R1=3,5-dimethylphenyl, R2=methyl, NR3R4=(1S,4R)-2,5-diazabicyclo[2.2.1]heptan-2-ylamino]

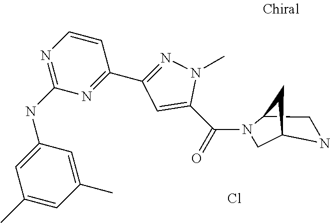 [SMILES: Cc1cc(C)cc(Nc2nccc(-c3cc(C(=O)N4C[C@H]5C[C@H]4CN5)n(C)n3)n2)c1 ; Cl]

¹H NMR (500 MHz, DMSO-d6) (mix of conformers) δ ppm 9.55 and 9.54 (2×s, 1H), 9.45 and 9.38 (2×br.s., 1H), 9.07 and 8.97 (2×br.s., 1H), 8.51 and 8.50 (2×d, J=5.2 Hz, 1H), 7.48 and 7.45 (2×s, 2H), 7.30 and 7.27 (2×d, J=5.2 Hz, 1H), 7.18 and 7.12 (2×S, 1H), 6.61 (s, 1H), 4.87 and 4.82 (2×s, 1H), 4.50 and 4.44 (2×s, 1H), 4.09 and 4.02 (2×s, 3H), 3.95-3.24 (m, 4H), 2.26 (s, 6H), 2.22-1.80 (m, 2H); HRMS (ESI) calcd for C22H26N7OCl [M+H]+ 404.2194, found 404.2199.

### 3-{2-[(3,5-dimethylphenyl)amino]pyrimidin-4-yl}-1-methyl-N-[(3R)-piperidin-3-yl]-1H-pyrazole-5-carboxamide hydrochloride (cpd 97) [(I), R1=3,5-dimethylphenyl, R2=methyl, R3=(3)-piperidin-3-yl, R4=H]

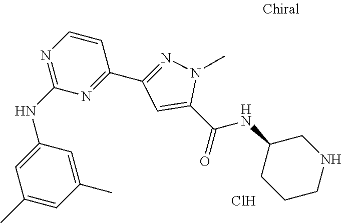 [SMILES: Cc1cc(C)cc(Nc2nccc(-c3cc(C(=O)N[C@@H]4CCCNC4)n(C)n3)n2)c1 ; Cl]

¹H NMR (500 MHz, DMSO-d6) δ ppm 9.49 (s, 1H), 8.85 (m, 4H), 8.50 (d, J=5.19 Hz, 1H), 7.52 (s, 1H), 7.47 (s, 2H), 7.26 (d, J=5.03 Hz, 1H), 6.62 (s, 1H), 4.14 (s, 3H), 3.42-2.74 (m. br., 5H) 2.27 (s, 6H), 1.98-1.53 (m. br., 4H); HRMS (ESI) calcd for C22H28N7OCl [M+H]+ 406.235, found 406.235.

### N-[(1R,2S)-2-aminocyclohexyl]-3-{2-[(3,5-dimethylphenyl)amino]pyrimidin-4-yl}-1-methyl-1H-pyrazole-5-carboxamide hydrochloride (cpd 98) [(I), R1=3,5-dimethylphenyl, R2=methyl, R3=(1R,2S)-2-aminocyclohexyl, R4=H]

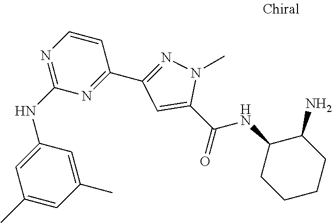 [SMILES: Cc1cc(C)cc(Nc2nccc(-c3cc(C(=O)N[C@@H]4CCCC[C@@H]4N)n(C)n3)n2)c1]

¹H NMR (500 MHz, DMSO-d6) δ ppm 9.51 (s, 1H), 8.50 (d, J=5.03 Hz, 1H), 8.47 (d, J=8.08 Hz, 1H), 7.87 (d, J=3.66 Hz, 3H), 7.60 (s, 1H), 7.51 (s, 2H), 7.27 (d, J=5.03 Hz, 1H), 6.62 (s, 1H), 4.33 (br. s., 1H), 4.14 (s, 3H), 3.35 (m overlapped by water signal, 1H), 2.28 (s, 6H), 185-1.30 (m. 8H); HRMS (ESI) calcd for C23H30N7OCl [M+H]+ 420.2507, found 420.2514.

### N-[(1S,2R)-2-aminocyclohexyl]-3-{2-[(3,5-dichlorophenyl)amino]pyrimidin-4-yl}-1-methyl-1H-pyrazole-5-carboxamide hydrochloride (cpd 99) [(I), R1=(3,5-dichlorophenyl), R2=methyl, R3=(1S,2R)-2-aminocyclohexyl, R4=H]

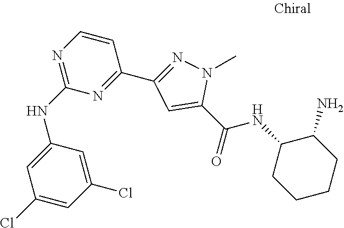 [SMILES: Cn1nc(-c2ccnc(Nc3cc(Cl)cc(Cl)c3)n2)cc1C(=O)N[C@H]1CCCC[C@H]1N]

¹H NMR (500 MHz, DMSO-d6) δ ppm 10.12 (s, 1H), 8.62 (d, J=5.19 Hz, 1H), 8.43 (d, J=7.78 Hz, 1H), 8.02 (d, J=1.83 Hz, 2H), 7.90 (d, J=3.51 Hz, 3H), 7.59 (s, 1H), 7.40 (d, J=5.03 Hz, 1H), 7.15 (t, J=1.83 Hz, 1H), 4.32 (br. s., 1H), 4.14 (s, 3H), 3.48-3.40 (m overlapped with water signal, 1H), 1.86-1.32 (m, 8H); HRMS (ESI) calcd for C21H24N7OCl3 [M+H]+ 460.1414, found 460.1419.

### N-[(1S,2R)-2-aminocyclohexyl]-3-{2-[(3,5-dimethoxyphenyl)amino]pyrimidin-4-yl}-1-methyl-1H-pyrazole-5-carboxamide hydrochloride (cpd 100) [(I), R1=3,5-dimethoxyphenyl, R2=methyl, R3=(1S,2R)-2-aminocyclohexyl, R4=H]

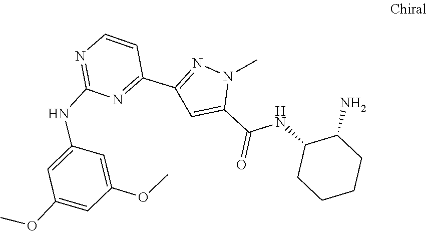 [SMILES: COc1cc(Nc2nccc(-c3cc(C(=O)N[C@H]4CCCC[C@H]4N)n(C)n3)n2)cc(OC)c1]

¹H NMR (500 MHz, DMSO-d6) δ ppm 9.62 (s, 1H), 8.53 (d, J=5.03 Hz, 1H), 8.43 (d, J=7.78 Hz, 1H), 7.89 (d, J=3.36 Hz, 3H), 7.60 (s, 1H), 7.31 (d, J=5.19 Hz, 1H), 7.20 (d, J=2.14 Hz, 2H), 6.13 (t, J=2.21 Hz, 1H), 4.31 (br. s., 1H), 4.13 (s, 3H), 3.76 (s, 6H), 3.48-3.40 (m overlapped with water signal, 1H), 1.88-1.33 (m, 8H); HRMS (ESI) calcd for C23H30N7O3 Cl [M+H]+ 452.2405, found 452.2396.

### N-[(1S,2S)-2-aminocyclohexyl]-3-{2-[(3,5-dimethylphenyl)amino]pyrimidin-4-yl}-1-methyl-1H-pyrazole-5-carboxamide (cpd 101) [(I), R1=3,5-dimethylphenyl, R2=methyl, R3=(1S,2S)-2-aminocyclohexyl, R4=H]

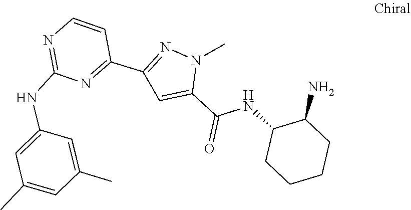 [SMILES: Cc1cc(C)cc(Nc2nccc(-c3cc(C(=O)N[C@H]4CCCC[C@@H]4N)n(C)n3)n2)c1]

¹H NMR (500 MHz, DMSO-d6) δ ppm 9.47 (s, 1H), 8.52 (d, J=8.39 Hz, 1H), 8.49 (d, J=5.03 Hz, 1H), 7.49 (s, 1H), 7.48 (s, 2H), 7.25 (d, J=5.03 Hz, 1H), 6.61 (s, 1H), 4.13 (s, 3H), 3.40-3.50 (m, 1H), 2.54 (td, J=10.90, 4.10 Hz, 1H), 2.27 (s, 6H), 1.90-1.17 (m, 8H); HRMS (ESI) calcd for C23H29N7O [M+H]+ 420.2507, found 420.2515.

### N-[(2S)-1-(azetidin-1-yl)-3-methylbutan-2-yl]-3-{2-[(3,5-dimethylphenyl)amino]pyrimidin-4-yl}-1-methyl-1H-pyrazole-5-carboxamide (cpd 102) [(I), R1=3,5-dimethylphenyl, R2=methyl, R3=(2S)-1-(azetidin-1-yl)-3-methylbutan-2-yl, R4=H]

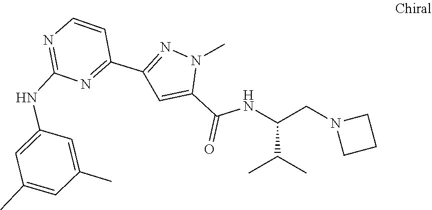 [SMILES: Cc1cc(C)cc(Nc2nccc(-c3cc(C(=O)N[C@H](CN4CCC4)C(C)C)n(C)n3)n2)c1]

¹H NMR (500 MHz, DMSO-d6) δ ppm 9.49 (s, 1H), 8.85 (d, J=7.78 Hz, 1H), 8.50 (d, J=5.19 Hz, 1H), 7.52 (s, 1H), 7.47 (s, 2H), 7.26 (d, J=5.03 Hz, 1H), 6.62 (s, 1H), 4.14 (s, 3H), 4.13-4.09 (m. br., 1H), 3.90-3.20 (m overlapped with water signal, 6H) (2.27 (s, 6H), 2.17-2.07 (m. br., 2H), 1.85-1.74 (m, 1H) 0.93-0.85 (m, 6H); HRMS (ESI) calcd for C25H33N7O [M+H]+ 448.282, found 448.2822.

### 3-{2-[(3,5-dimethylphenyl)amino]pyrimidin-4-yl}-1-methyl-N-[(2S)-3-methyl-1-(pyrrolidin-1-yl)butan-2-yl]-1H-pyrazole-5-carboxamide (cpd 103) [(I), R1=3,5-dimethylphenyl, R2=methyl, R3=(2S)-3-methyl-1-pyrrolidin-1-ylbutan-2-yl, R4=H]

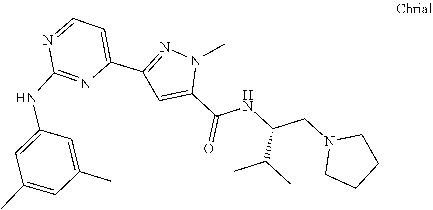 [SMILES: Cc1cc(C)cc(Nc2nccc(-c3cc(C(=O)N[C@H](CN4CCCC4)C(C)C)n(C)n3)n2)c1]

¹H NMR (500 MHz, DMSO-d6) δ ppm 9.49 (s, 1H), 8.49 (d, J=5.0 Hz, 1H), 8.41 (d, J=9.0 Hz, 1H), 7.50 (s, 2H), 7.48 (s, 1H), 7.27 (d, J=5.0 Hz, 1H), 6.61 (s, 1H), 4.11 (s, 3H), 4.03-3.92 (m, 1H), 2.59 (dd, J=9.1, 12.1 Hz, 1H), 2.53-2.47 (m. overlapped with DMSO signal, 2H) 2.47 (dd, J=4.7, 12.2 Hz, 1H), 2.40 (s, 2H), 2.27 (s, 6H), 1.94-1.77 (m, 1H), 1.65 (br. s., 4H), 0.90 (d, J=8.2 Hz, 3H), 0.89 (d, J=8.4 Hz, 3H); HRMS (ESI) calcd for C26H35N7O [M+H]+ 462.2976, found 462.2983.

### N-[(1S,2R)-2-aminocyclohexyl]-3-{2-[(3,5-dimethylphenyl)amino]pyrimidin-4-yl}-1H-pyrazole-5-carboxamide hydrochloride (cpd 104) [(I), R1=3,5-dimethylphenyl, R2=H, R3=(1S,2R)-2-aminocyclohexyl, R4=H]

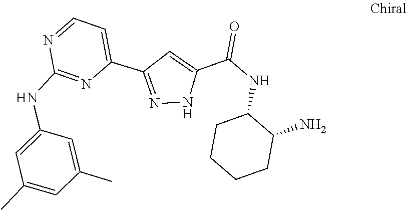 [SMILES: Cc1cc(C)cc(Nc2nccc(-c3cc(C(=O)N[C@H]4CCCC[C@H]4N)[nH]n3)n2)c1]

¹H NMR (500 MHz, DMSO-d6) δ ppm 14.5-13.5 (br. s., 1H), 9.49 (s, 1H), 8.53 (d, J=5.19 Hz, 1H), 8.21 (br. s., 1H), 7.90 (d, J=2.90 Hz, 3H), 7.54 (br. s., 1H), 7.48 (s, 2H), 7.34 (d, J=5.03 Hz, 1H), 6.63 (s, 1H), 4.33 (br. s., 1H), 3.50-3.40 (m overlapped with water signal, 1H), 2.27 (s, 6H), 1.90-1.17 (m, 8H); HRMS (ESI) calcd for C22H28N7OCl [M+H]+ 406.235, found 406.236.

### N-[(2S)-1-(azetidin-1-yl)-3-methylbutan-2-yl]-3-{2-[(3,5-dimethylphenyl)amino]pyrimidin-4-yl}-1H-pyrazole-5-carboxamide (cpd 105) [(I), R1=3,5-dimethylphenyl, R2=H, R3=(2S)-1-(azetidin-1-yl)-3-methylbutan-2-yl, R4=H]

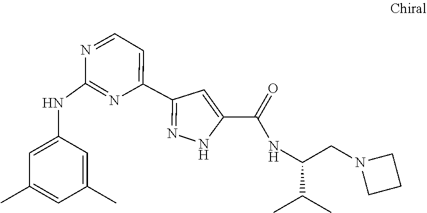 [SMILES: Cc1cc(C)cc(Nc2nccc(-c3cc(C(=O)N[C@H](CN4CCC4)C(C)C)[nH]n3)n2)c1]

¹H NMR (500 MHz, DMSO-d6) δ ppm 13.98 (br. s., 1H), 9.45 (s, 1H), 8.51 (d, J=3.66 Hz, 1H), 8.22 (br. s., 1H), 7.48 (s, 3H), 7.31 (d, J=5.03 Hz, 1H), 6.62 (s, 1H), 3.70-3.82 (m, 1H), 3.20-3.05 (m. 4H), 3.17 (d, J=4.88 Hz, 2H), 2.27 (s, 6H), 1.92 (quin, J=6.63 Hz, 2H), 1.80 (dq, J=13.17, 6.54 Hz, 1H), 0.87 (dd, J=9.76, 6.86 Hz, 6H); HRMS (ESI) calcd for C24H31N7O [M+H]+ 434.2663, found 434.2668.

### N-[(1S)-1-cyclohexyl-2-hydroxyethyl]-3-{2-[(3,5-dimethylphenyl)amino]pyrimidin-4-yl}-1-methyl-1H-pyrazole-5-carboxamide (cpd 106) [(I), R1=3,5-dimethylphenyl, R2=methyl, R3=(1S)-1-cyclohexyl-2-hydroxyethyl, R4=H]

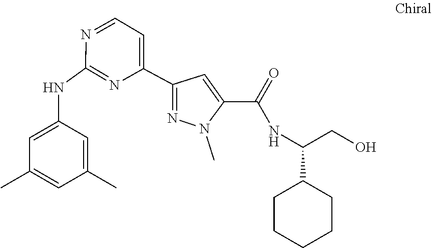 [SMILES: Cc1cc(C)cc(Nc2nccc(-c3cc(C(=O)N[C@H](CO)C4CCCCC4)n(C)n3)n2)c1]

¹H NMR (500 MHz, DMSO-d6) δ ppm 9.49 (s, 1H), 8.49 (d, J=5.19 Hz, 1H), 8.37 (d, J=9.00 Hz, 1H), 7.51 (s, 1H), 7.50 (s, 2H), 7.26 (d, J=5.19 Hz, 1H), 6.61 (s, 1H), 4.57 (t, J=5.72 Hz, 1H), 4.12 (s, 3H), 3.74-3.86 (m, 1H), 3.50-3.58 (m, 1H), 3.44-3.50 (m, 1H), 2.27 (s, 6H), 1.80-0.95 (m. 11H); HRMS (ESI) calcd for C25H32N6O2 [M+H]+ 449.266, found 449.2665.

### 3-{2-[(3,5-dimethylphenyl)amino]pyrimidin-4-yl}-1-methyl-N-[(1S)-1-phenyl-2-(pyrrolidin-1-yl)ethyl]-1H-pyrazole-5-carboxamide (cpd 107) [(I), R1=3,5-dimethylphenyl, R2=methyl, R3=(1S)-1-phenyl-2-pyrrolidin-1-ylethyl, R4=H]

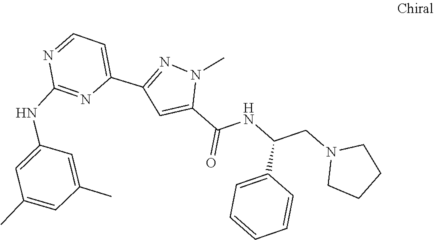 [SMILES: Cc1cc(C)cc(Nc2nccc(-c3cc(C(=O)N[C@H](CN4CCCC4)c4ccccc4)n(C)n3)n2)c1]

¹H NMR (500 MHz, DMSO-d6) δ ppm 9.50 (s, 1H), 9.15 (d, J=8.4 Hz, 1H), 8.50 (d, J=5.0 Hz, 1H), 7.60 (s, 1H), 7.51 (s, 2H), 7.43 (d, J=7.3 Hz, 2H), 7.33 (t, J=7.5 Hz, 2H), 7.28-7.25 (m, 2H), 6.63 (s, 1H), 5.19-5.10 (m, 1H), 4.10 (s, 3H), 2.97 (t, J=10.9 Hz, 2H), 2.60-2.48 (m, 4H), 2.30 (s, 6H), 1.67 (br. s., 4H); HRMS (ESI) calcd for C29H33N7O [M+H]+ 496.282, found 496.2823.

### N-[(2S)-1-(azetidin-1-yl)propan-2-yl]-1-methyl-3-{2-[(3-methylphenyl)amino]pyrimidin-4-yl}-1H-pyrazole-5-carboxamide (cpd 108) [(I), R1=3-methylphenyl, R2=methyl, R3=(2S)-1-(azetidin-1-yl)propan-2-yl, R4=H]

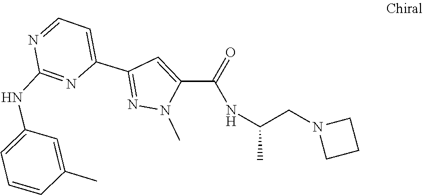 [SMILES: Cc1cccc(Nc2nccc(-c3cc(C(=O)N[C@@H](C)CN4CCC4)n(C)n3)n2)c1]

¹H NMR (500 MHz, DMSO-d6) δ ppm 9.55 (s, 1H), 8.50 (d, J=5.19 Hz, 1H), 8.46 (d, J=8.39 Hz, 1H), 7.69 (s, 1H), 7.63 (d, J=8.24 Hz, 1H), 7.46 (s, 1H), 7.27 (d, J=5.03 Hz, 1H), 7.18 (t, J=7.78 Hz, 1H), 6.79 (d, J=7.63 Hz, 1H), 4.14 (s, 3H), 3.92 (dquin, J=13.93, 6.77, 6.77, 6.77, 6.77 Hz, 1H), 3.38-3.29 (m. overlapped with water signal, 2H), 3.04-3.24 (m, 4H), 2.32 (s, 3H), 2.00-1.90 (m. 2H), 1.10 (d, J=6.71 Hz, 3H); HRMS (ESI) calcd for C22H27N7O [M+H]+ 406.235, found 406.2349.

### 3-{2-[(3,5-dimethylphenyl)amino]pyrimidin-4-yl}-N-[(2S)-1-hydroxy-3-phenylpropan-2-yl]-1-methyl-1H-pyrazole-5-carboxamide (cpd 109) [(I), R1=3,5-dimethylphenyl, R2=methyl, R3=(2S)-1-hydroxy-3-phenylpropan-2-yl, R4=H]

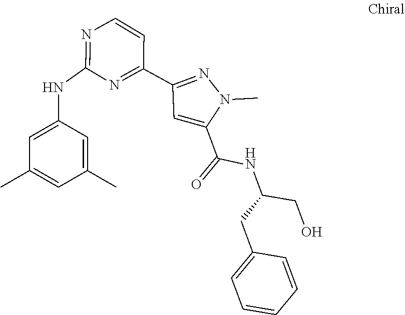 [SMILES: Cc1cc(C)cc(Nc2nccc(-c3cc(C(=O)N[C@H](CO)Cc4ccccc4)n(C)n3)n2)c1]

¹H NMR (500 MHz, DMSO-d6) δ ppm 9.47 (s, 1H), 8.55 (d, J=8.5 Hz, 1H), 8.49 (d, J=5.2 Hz, 1H), 7.48 (s, 2H), 7.44 (s, 1H), 7.31-7.12 (m, 6H), 6.62 (s, 1H), 4.87 (t, J=5.6 Hz, 1H), 4.18-4.08 (m, 1H), 4.04 (s, 3H), 3.54-3.46 (m, 1H), 3.46-3.40 (m, 1H), 2.95 (dd, J=4.7, 13.7 Hz, 1H), 2.73 (dd, J=9.5, 13.9 Hz, 1H), 2.29 (s, 6H); HRMS (ESI) calcd for C26H28N6O2 [M+H]+ 457.2347, found 457.2354.

### 3-(2-{[3-chloro-4-(4-methylpiperazin-1-yl)phenyl]amino}pyrimidin-4-yl)-N-[(2S)-1-hydroxypropan-2-yl]-1-methyl-1H-pyrazole-5-carboxamide (cpd 110) [(I), R1=3-chloro-4-(4-methylpiperazin-1-yl)phenyl, R2=methyl, R3=(2S)-1-hydroxypropan-2-yl, R4=H]

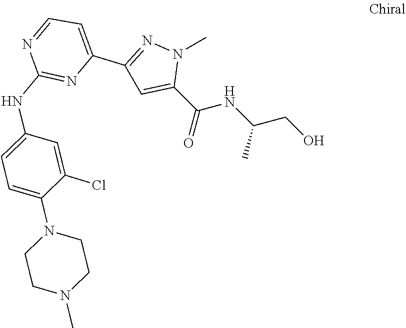 [SMILES: C[C@@H](CO)NC(=O)c1cc(-c2ccnc(Nc3ccc(N4CCN(C)CC4)c(Cl)c3)n2)nn1C]

¹H NMR (500 MHz, DMSO-d6) δ ppm 9.67 (s, 1H), 8.51 (d, J=5.03 Hz, 1H), 8.41 (d, J=8.08 Hz, 1H), 7.95 (d, J=2.44 Hz, 1H), 7.76 (dd, J=8.77, 2.36 Hz, 1H), 7.47 (s, 1H), 7.27 (d, J=5.19 Hz, 1H), 7.14 (d, J=8.85 Hz, 1H), 4.76 (t, J=5.80 Hz, 1H), 4.13 (s, 3H), 3.94-4.03 (m, 1H), 3.45-3.36 (m, 2H), 2.93 (br. s., 4H), 2.54-2.43 (m. br, partially overlapped with water, 4H), 2.23 (s, 3H) 1.14 (d, J=6.71 Hz, 3H); HRMS (ESI) calcd for C23H29N8O2 Cl [M+H]+ 485.2175, found 485.2172.

### 3-(2-{[3-chloro-4-(4-methylpiperazin-1-yl)phenyl]amino}pyrimidin-4-yl)-N-[(2S)-1-hydroxy-3-methylbutan-2-yl]-1-methyl-1H-pyrazole-5-carboxamide (cpd 111) [(I), R1=3-chloro-4-(4-methylpiperazin-1-yl)phenyl, R2=methyl, R3=(2S)-1-hydroxy-3-methylbutan-2-yl, R4=H]

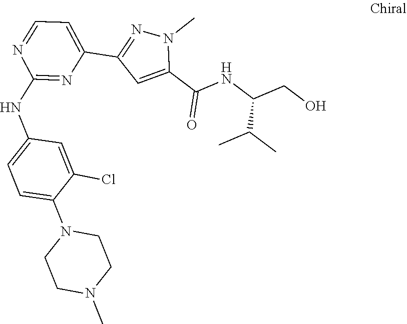 [SMILES: CC(C)[C@@H](CO)NC(=O)c1cc(-c2ccnc(Nc3ccc(N4CCN(C)CC4)c(Cl)c3)n2)nn1C]

¹H NMR (500 MHz, DMSO-d6) δ ppm 9.69 (s, 1H), 8.51 (d, J=5.2 Hz, 1H), 8.31 (d, J=9.0 Hz, 1H), 8.02 (d, J=2.6 Hz, 1H), 7.70 (dd, J=2.5, 8.8 Hz, 1H), 7.51-7.46 (m, 1H), 7.28 (d, J=5.2 Hz, 1H), 7.12 (d, J=8.8 Hz, 1H), 4.61 (t, J=5.6 Hz, 1H), 4.12 (s, 3H), 3.84-3.74 (m, 1H), 3.60-3.44 (m, 2H), 2.92 (br. s., 4H), 2.52-2.40 (br. s., partially overlapped with water, 4H) 2.23 (s, 3H), 1.98-1.87 (m, 1H), 0.91 (dd, J=6.8, 9.7 Hz, 6H); HRMS (ESI) calcd for C25H33N8O2 Cl [M+H]+ 513.2488, found 513.2498.

### 1-methyl-3-{2-[(3-methylphenyl)amino]pyrimidin-4-yl}-N-[(2S)-3-methyl-1-(pyrrolidin-1-yl)butan-2-yl]-1H-pyrazole-5-carboxamide (cpd 112) [(I), R1=3-methylphenyl, R2=methyl, R3=(2S)-3-methyl-1-pyrrolidin-1-ylbutan-2-yl, R4=H]

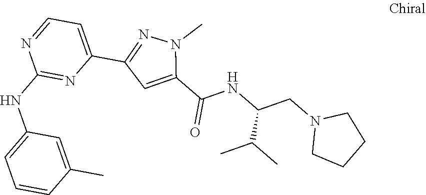 [SMILES: Cc1cccc(Nc2nccc(-c3cc(C(=O)N[C@H](CN4CCCC4)C(C)C)n(C)n3)n2)c1]

¹H NMR (500 MHz, DMSO-d6) δ ppm 9.56 (s, 1H), 8.50 (d, J=5.2 Hz, 1H), 8.39 (d, J=9.0 Hz, 1H), 7.79 (s, 1H), 7.56 (d, J=8.4 Hz, 1H), 7.50-7.47 (m, 1H), 7.28 (d, J=5.0 Hz, 1H), 7.16 (t, J=7.8 Hz, 1H), 6.79 (d, J=7.5 Hz, 1H), 4.12 (s, 3H), 3.98 (td, J=4.4, 9.3 Hz, 1H), 2.63-2.57 (m, 1H), 2.52-2.47 (br. m., partially overlapped with water, 3H), 2.44-2.38 (m, 2H), 2.32 (s, 3H), 1.86 (dd, J=6.6, 12.4 Hz, 1H), 1.65 (br. s., 4H), 0.90 (t, J=7.3 Hz, 6H); HRMS (ESI) calcd for C25H33N7O [M+H]+ 448.282, found 448.2827.

### N-[(1S,2S)-2-aminocyclohexyl]-3-{2-[(3,5-difluorophenyl)amino]pyrimidin-4-yl}-1-methyl-1H-pyrazole-5-carboxamide (cpd 113) [(I), R1=3,5-difluorophenyl, R2=methyl, R3=(1S,2S)-2-aminocyclohexyl, R4=H]

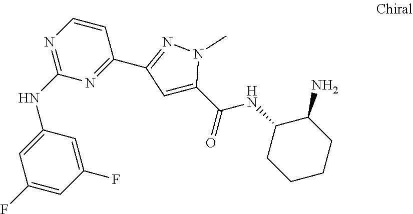 [SMILES: Cn1nc(-c2ccnc(Nc3cc(F)cc(F)c3)n2)cc1C(=O)N[C@H]1CCCC[C@@H]1N]

¹H NMR (500 MHz, DMSO-d6) δ ppm 10.11 (s, 1H), 8.59 (d, J=5.03 Hz, 1H), 8.49 (d, J=8.39 Hz, 1H), 7.62 (dd, J=10.45, 2.21 Hz, 2H), 7.50 (s, 1H), 7.38 (d, J=5.03 Hz, 1H), 6.76 (tt, J=9.21, 2.31 Hz, 1H), 4.14 (s, 3H), 3.41-3.50 (m, 1H), 2.55 (td, J=10.50, 3.50 Hz, 1H), 1.93-1.18 (m, 8H); HRMS (ESI) calcd for C21H23N7OF2 [M+H]+428.2005, found 428.2004.

### 3-(2-{[3-chloro-4-(4-methylpiperazin-1-yl)phenyl]amino}pyrimidin-4-yl)-N-[(1S,2R)-2-hydroxycyclohexyl]-1-methyl-1H-pyrazole-5-carboxamide (cpd 114) [(I), R1=3-chloro-4-(4-methylpiperazin-1-yl)phenyl, R2=methyl, R3=(1S,2R)-2-hydroxycyclohexyl, R4=H]

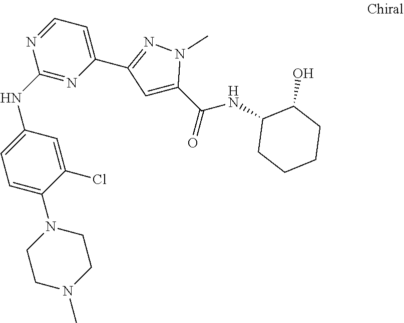 [SMILES: CN1CCN(c2ccc(Nc3nccc(-c4cc(C(=O)N[C@H]5CCCC[C@H]5O)n(C)n4)n3)cc2Cl)CC1]

¹H NMR (500 MHz, DMSO-d6) δ ppm 9.69 (s, 1H), 8.51 (d, J=5.18 Hz, 1H), 8.13 (d, J=7.78 Hz, 1H), 7.97 (d, J=2.59 Hz, 1H), 7.76 (dd, J=8.69, 2.44 Hz, 1H), 7.49 (s, 1H), 7.28 (d, J=5.19 Hz, 1H), 7.13 (d, J=8.85 Hz, 1H), 4.68 (d, J=3.81 Hz, 1H), 4.13 (s, 3H), 3.78-3.92 (m, 2H), 2.92 (br. s., 4H), 2.54-2.42 (m. br., partially overlapped with water, 4H), 2.23 (s, 3H), 1.78-1.15 (m, 8H); HRMS (ESI) calcd for C26H33N8O2 Cl [M+H]+ 525.2488, found 525.2492.

### 3-{2-[(3,5-dimethylphenyl)amino]pyrimidin-4-yl}-N-[(1S,2R)-2-hydroxycyclohexyl]-1-methyl-1H-pyrazole-5-carboxamide (cpd 115) [(I), R1=3,5-dimethylphenyl, R2=methyl, R3=(1S,2R)-2-hydroxycyclohexyl, R4=H]

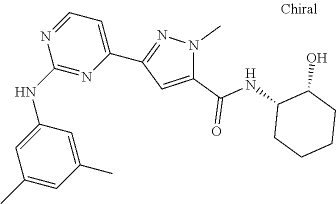 [SMILES: Cc1cc(C)cc(Nc2nccc(-c3cc(C(=O)N[C@H]4CCCC[C@H]4O)n(C)n3)n2)c1]

¹H NMR (500 MHz, DMSO-d6) δ ppm 9.48 (s, 1H), 8.49 (d, J=5.03 Hz, 1H), 8.16 (d, J=7.78 Hz, 1H), 7.49 (s, 1H), 7.48 (s, 2H), 7.25 (d, J=5.19 Hz, 1H), 6.61 (s, 1H), 4.64 (d, J=3.81 Hz, 1H), 4.12 (s, 3H), 3.78-3.90 (m, 2H), 2.27 (s, 6H), 1.84-1.17 (m, 8H); HRMS (ESI) calcd for C23H28N6O2 [M+H]+ 421.2347, found 421.2345.

### N-[(1S,2S)-2-aminocyclohexyl]-3-{2-[(3,5-dichlorophenyl)amino]pyrimidin-4-yl}-1-methyl-1H-pyrazole-5-carboxamide (cpd 116) [(I), R1=3,5-dichlorophenyl, R2=methyl, R3=(1S,2S)-2-aminocyclohexyl, R4=H]

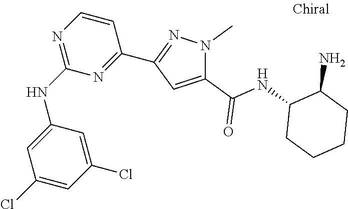 [SMILES: Cn1nc(-c2ccnc(Nc3cc(Cl)cc(Cl)c3)n2)cc1C(=O)N[C@H]1CCCC[C@@H]1N]

¹H NMR (500 MHz, DMSO-d6) δ ppm 10.10 (s, 1H), 8.62-8.59 (m, 1H), 8.49 (d, J=8.4 Hz, 1H), 8.00-7.97 (m, 2H), 7.47 (s, 1H), 7.38 (d, J=5.0 Hz, 1H), 7.13 (t, J=1.8 Hz, 1H), 4.15-4.11 (m, 3H), 3.49-3.41 (m, 1H), 2.59-2.52 (m, 1H), 1.88-1.09 (m. 8H); HRMS (ESI) calcd for C21H23N7OCl2 [M+H]+ 460.1414, found 460.1427.

### 3-{2-[(3,5-dichlorophenyl)amino]pyrimidin-4-yl}-1-methyl-N-[(2S)-3-methyl-1-(pyrrolidin-1-yl)butan-2-yl]-1H-pyrazole-5-carboxamide (cpd 117) [(I), R1=3,5-dichlorophenyl, R2=methyl, R3=(2S)-3-methyl-1-pyrrolidin-1-ylbutan-2-yl, R4=H]

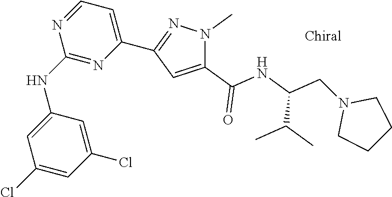 [SMILES: CC(C)[C@@H](CN1CCCC1)NC(=O)c1cc(-c2ccnc(Nc3cc(Cl)cc(Cl)c3)n2)nn1C]

¹H NMR (500 MHz, DMSO-d6) δ ppm 10.10 (s, 1H), 8.60 (d, J=5.0 Hz, 1H), 8.37 (d, J=9.2 Hz, 1H), 8.00 (d, J=1.8 Hz, 2H), 7.45 (s, 1H), 7.40 (d, J=5.0 Hz, 1H), 7.13 (t, J=1.8 Hz, 1H), 4.17-4.08 (m, 3H), 4.03-3.93 (m, 1H), 2.63-2.54 (m, 2H), 2.44-2.36 (m, 4H), 1.86 (dd, J=6.8, 12.4 Hz, 1H), 1.65 (br. s., 4H), 0.90 (dd, J=7.1, 7.9 Hz, 6H); HRMS (ESI) calcd for C24H29N7OCl2 [M+H]+ 502.1884, found 502.1893.

### 3-{2-[(3,5-dimethylphenyl)amino]pyrimidin-4-yl}-N-[(1S,2S)-2-hydroxycyclohexyl]-1-methyl-1H-pyrazole-5-carboxamide (cpd 118) [(I), R1=3,5-dimethylphenyl, R2=methyl, R3=(1S,2S)-2-hydroxycyclohexyl, R4=H]

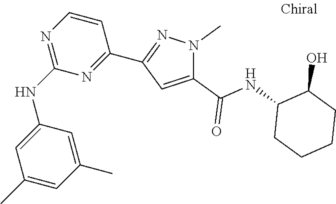 [SMILES: Cc1cc(C)cc(Nc2nccc(-c3cc(C(=O)N[C@H]4CCCC[C@@H]4O)n(C)n3)n2)c1]

¹H NMR (500 MHz, DMSO-d6) δ ppm 9.46 (s, 1H), 8.49 (d, J=5.0 Hz, 1H), 8.46 (d, J=8.2 Hz, 1H), 7.47 (s, 3H), 7.25 (d, J=5.2 Hz, 1H), 6.61 (s, 1H), 4.61 (d, J=5.2 Hz, 1H), 4.13 (s, 3H), 3.66-3.53 (m, 1H), 3.42-3.28 (m, overlapped with water, 1H), 2.27 (s, 6H), 1.97-1.76 (m, 2H), 1.64 (br. s, 2H), 1.35-1.13 (m, 4H); HRMS (ESI) calcd for C23H28N6O2 [M+H]+ 421.2347, found 421.235.

### N-[(2S)-1-(azetidin-1-yl)propan-2-yl]-3-{2-[(3-cyanophenyl)amino]pyrimidin-4-yl}-1-methyl-1H-pyrazole-5-carboxamide formate (cpd 119) [(I), R1=3-cyanophenyl, R2=methyl, R3=(2S)-1-(azetidin-1-yl)propan-2-yl, R4=H]

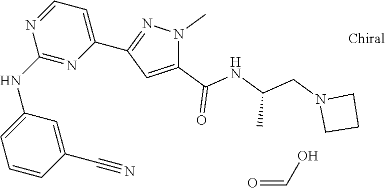

¹H NMR (500 MHz, DMSO-d6) δ ppm 10.04 (s, 1H), 8.59 (d, J=5.0 Hz, 1H), 8.44 (d, J=8.4 Hz, 1H), 8.31 (t, J=1.7 Hz, 1H), 8.28 (s, 1H), 8.14 (dd, J=1.2, 8.4 Hz, 1H), 7.52 (t, J=8.0 Hz, 1H), 7.47 (s, 1H), 7.41 (d, J=7.8 Hz, 1H), 7.36 (d, J=5.0 Hz, 1H), 4.14 (s, 3H), 3.99-3.84 (m, 1H), 3.24-3.09 (m, 4H), 2.55-2.48 (m, partially overlapped with water 1H) 2.43-2.38 (m, 1H), 1.95 (quin, J=7.0 Hz, 2H), 1.11 (d, J=6.7 Hz, 3H); HRMS (ESI) calcd for C23H26N8O3 [M+H]+ 417.2146, found 417.2161.

### 3-{2-[(3,5-dimethylphenyl)amino]pyrimidin-4-yl}-1-ethyl-N-[(1S,2R)-2-hydroxycyclohexyl]-1H-pyrazole-5-carboxamide (cpd 120) [(I), R1=3,5-dimethylphenyl, R2=ethyl, R3=(1S,2R)-2-hydroxycyclohexyl, R4=H]

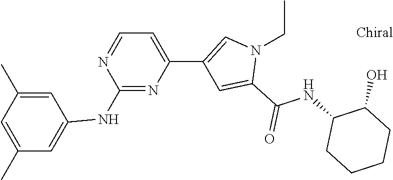 [SMILES: CCn1cc(-c2ccnc(Nc3cc(C)cc(C)c3)n2)cc1C(=O)N[C@H]1CCCC[C@H]1O]

¹H NMR (500 MHz, DMSO-d6) δ ppm 9.49 (s, 1H), 8.49 (d, J=5.19 Hz, 1H), 8.16 (d, J=7.63 Hz, 1H), 7.49 (s, 2H), 7.48 (s, 1H), 7.26 (d, J=5.19 Hz, 1H), 6.61 (s, 1H), 4.64 (d, J=3.97 Hz, 1H), 4.54 (q, J=7.17 Hz, 2H), 3.79-3.90 (m, 2H), 2.26 (s, 6H), 1.80-1.28 (m, 8H), 1.38 (t, J=7.17 Hz, 3H); HRMS (ESI) calcd for C24H30N6O2 [M+H]+435.2503, found 435.2502.

### N-[(1S,2R)-2-aminocyclohexyl]-3-{2-[(3,5-dimethylphenyl)amino]pyrimidin-4-yl}-1-ethyl-1H-pyrazole-5-carboxamide hydrochloride (cpd 121) [(I), R1=3,5-dimethylphenyl, R2=ethyl, R3=(1S,2R)-2-aminocyclohexyl, R4=H]

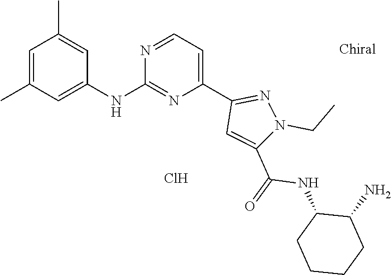 [SMILES: CCn1nc(-c2ccnc(Nc3cc(C)cc(C)c3)n2)cc1C(=O)N[C@H]1CCCC[C@H]1N ; Cl]

¹H NMR (500 MHz, DMSO-d6) δ ppm 9.51 (s, 1H), 8.51 (d, J=5.03 Hz, 1H), 8.46 (d, J=7.93 Hz, 1H), 7.88 (d, J=4.27 Hz, 3H), 7.61 (s, 1H), 7.52 (s, 2H), 7.28 (d, J=5.03 Hz, 1H), 6.62 (s, 1H), 4.66-4.45 (m, 2H), 4.34 (br. s., 1H), 3.74-3.50 (m, 1H), 2.28 (s, 6H), 1.88-1.31 (m, 8H), 1.42 (t, J=7.17 Hz, 3H); HRMS (ESI) calcd for C24H32N7OCl [M+H]+ 434.2663, found 434.2658.

### 3-{2-[(3,5-dimethylphenyl)amino]pyrimidin-4-yl}-1-ethyl-N-[(3R)-piperidin-3-yl]-1H-pyrazole-5-carboxamide hydrochloride (cpd 122) [(I), R1=3,5-dimethylphenyl, R2=ethyl, R3=(3R)-piperidin-3-yl, R4=H]

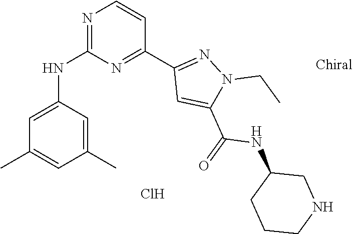 [SMILES: CCn1nc(-c2ccnc(Nc3cc(C)cc(C)c3)n2)cc1C(=O)N[C@@H]1CCCNC1 ; Cl]

¹H NMR (500 MHz, DMSO-d6) δ ppm 9.50 (s, 1H), 8.95 (d, J=9.46 Hz, 1H), 8.86 (d, J=7.47 Hz, 1H), 8.78-8.89 (m, 1H), 8.50 (d, J=5.03 Hz, 1H), 7.52 (s, 1H), 7.48 (s, 2H), 7.27 (d, J=5.19 Hz, 1H), 6.62 (s, 1H), 4.56 (m, 2H), 4.09-4.22 (m, 1H), 3.40-2.73 (m, 4H) 2.27 (s, 6H), 1.95-1.55 (m. 4H), 1.39 (t, J=7.17 Hz, 3H); HRMS (ESI) calcd for C23H30N7OCl [M+H]+ 420.2507, found 420.2513.

### N-[(2S)-1-(azetidin-1-yl)propan-2-yl]-3-{2-[(3,5-dimethoxyphenyl)amino]pyrimidin-4-yl}-N,1-dimethyl-1H-pyrazole-5-carboxamide (cpd 123) [(I), R1=3,5-dimethoxyphenyl, R2=methyl, R3=(2S)-1-(azetidin-1-yl)propan-2-yl, R4=methyl]

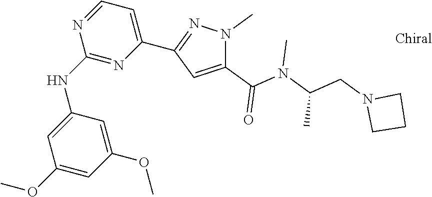 [SMILES: COc1cc(Nc2nccc(-c3cc(C(=O)N(C)[C@@H](C)CN4CCC4)n(C)n3)n2)cc(OC)c1]

¹H NMR (500 MHz, DMSO-d6) δ ppm 9.62 (s, 1H), 8.49-8.55 (m, 1H), 7.31 (d, J=5.03 Hz, 1H), 7.18 (m. 3H), 6.13 (br. s., 1H), 4.00 (m. 6H), 3.76-3.60 (m. br. 1H) 3.74 (s. 6H), 3.16-3.10 (m. 2H), 3.06-2.69 (m. br. 4H), 2.04-1.60 (m. br. 2H), 1.25 (m. 3H); HRMS (ESI) calcd for C24H31N7O3 [M+H]+ 466.2561, found 466.2564.

### Biochemical Kinase Inhibition Assay on SYK in KinaseGlo e ADPGlo Assay Formats

Recombinant proteins SYK FL was produced at NMS via baculo virus infection in insect cells as His GST-fusion protein. In-house protein preparation was >80% homogeneous as judged by SDS-PAGE and they were characterized by N-terminal sequence analysis and electrospray mass spectrometry.

SYK was pre-activated at the concentration of 1 μM with 400 μM ATP in kinase buffer (50 mM Hepes pH 7.5, 10 mM MgCl2, 1 mM DTT, 3 μM Na3VO4 and 0.2 mg/mL BSA) for 60 minute at 28° C. just before kinase reaction.

### KinaseGlo Assay Format

Compounds were 3-fold serially diluted from 10 to 0.0005 μM, then incubated for 60 minutes at rt in the presence of ATP 2.5 μM, peptidic substrate BioDBn*327 125 uM and preactivated enzyme 2.5 nM in a final volume of 20 μL of kinase buffer (50 mM Hepes pH 7.5, 10 mM MgCl2, 1 mM DTT, 3 μM Na3VO4 and 0.2 mg/mL BSA). The final concentration of DMSO was 1%. The assay was run in a robotized format on 384-well plates (Perkin Elmer cat. #6005301).

### ADPGlo Assay Format

Five microliters of test compounds 4-fold serially diluted from 10 to 0.0006 μM dissolved in 3% DMSO were pipetted into 384-well Optiplate (n* 6005310—Perkin Elmer). Five microliters of recombinant pre-activated SYK solution at 1.8 nM diluted in its specific kinase buffer was added to the compound-containing plate and was incubated for 30 minutes at room temperature. Five microliters of a mixture of ATP (Adenosine 5′-triphosphate, disodium salt (Promega)) and peptidic substrate BioDBn*327 (TwinHelix) diluted in kinase buffer were added to start the reactions. Final concentrations of pre-activated SYK, ATP and BioDBn*327 were 0.6 nM, 60 μM and 125 μM, respectively

The reaction mixtures were incubated for 60 minutes at room temperature and then stopped with the addition of 15 μL of Reagent 1 of ADPGlo® kit (V9102—Promega).

The ADPGlo® Reagent 2 was added after 60 minutes and then the luminescence signal of the plates was measured to the Pherastar plate reader (BMG).

### Data Analysis

Each 384-well plate contained at least one curve of a standard cpd, and reference wells (total enzyme activity vs enzyme completely inhibited) for the Z′ and signal to background evaluation (J. Biomol. Screening, 1999, 4, 67-73). All information about plate dilution, distribution and raw data of inhibition are tracked via barcode reading and stored in an Oracle DB. The data per each molecule are analyzed by an internally customized version of the SW package “Assay Explorer” which provides sigmoidal fittings of the eight-dilution curves for IC50 determination using a 4 parameter logistic equation:

y=bottom+(top−bottom)/(1+10\hat{ }((log IC50−x)*slope))
where x is the logarithm of the inhibitor concentration, y is the response;
IC50 is defined as the concentration of compound required to inhibit 50% of maximum phosphorylation.

Representative compounds of the invention of formula (I) were tested on Syk in the specific in vitro kinase assays above described.

Table A below, reports the in vitro activity data, performed as reported above, of the compounds of formula (I) against Syk kinase. As can be appreciated by the skilled person, most of the compounds show an IC₅₀ value <0.5 μM on Syk, and are thus particularly advantageous in therapy against diseases caused by and/or associated with dysregulated Syk kinase activity, such as cancer.

TABLE A

| | IC50 (μM) | IC50 (μM) |
|---|---|---|
| Example | Kglo | ADPGlo |
| 1 | 0.052 | |
| 2 | 0.394 | |
| 3 | 0.057 | |
| 4 | 0.111 | |
| 5 | 0.084 | |
| 6 | 0.072 | |
| 7 | 0.073 | |
| 8 | 0.282 | |
| 9 | 0.398 | |
| 11 | 0.485 | |
| 12 | 0.398 | |
| 13 | 0.226 | |
| 14 | 0.334 | |
| 15 | 0.358 | |
| 16 | 0.272 | |
| 17 | 0.060 | |
| 18 | 0.491 | |
| 19 | 0.104 | |
| 20 | 0.109 | |
| 21 | 0.238 | |
| 24 | 0.027 | |
| 25 | 0.249 | |
| 26 | 0.013 | |
| 27 | 0.230 | |
| 28 | 0.459 | |
| 31 | 0.139 | |
| 32 | 0.074 | |
| 33 | 0.017 | |
| 34 | 0.320 | |
| 35 | 0.024 | |
| 36 | 0.169 | |
| 37 | 0.009 | |
| 38 | 0.005 | |
| 39 | 0.021 | |
| 40 | 0.040 | |
| 41 | 0.077 | |
| 42 | 0.474 | |
| 43 | 0.364 | |
| 45 | 0.065 | |
| 46 | 0.049 | |
| 47 | 0.040 | |
| 48 | 0.006 | 0.023 |
| 53 | 0.015 | |
| 55 | 0.324 | |
| 56 | 0.081 | |
| 57 | 0.185 | |
| 58 | 0.059 | |
| 59 | <0.004 | |
| 60 | 0.005 | |
| 61 | 0.004 | |
| 62 | 0.008 | |
| 63 | 0.021 | |
| 64 | <0.004 | 0.011 |
| 65 | 0.009 | 0.038 |
| 66 | 0.036 | |
| 67 | <0.004 | 0.014 |
| 68 | 0.025 | |
| 69 | 0.008 | 0.01 |
| 70 | 0.032 | |
| 71 | 0.452 | |
| 72 | 0.034 | 0.087 |
| 75 | 0.273 | 0.473 |
| 78 | 0.015 | 0.033 |
| 79 | 0.007 | 0.026 |
| 80 | 0.044 | 0.081 |
| 82 | 0.025 | |
| 83 | 0.087 | |
| 84 | 0.241 | |
| 85 | 0.209 | |
| 86 | 0.471 | |
| 88 | 0.051 | |
| 89 | 0.219 | |
| 90 | 0.156 | |
| 91 | 0.035 | |
| 92 | 0.006 | |
| 93 | 0.137 | |
| 94 | 0.013 | |
| 95 | <0.004 | |
| 96 | 0.070 | |
| 97 | 0.048 | |
| 99 | 0.011 | |
| 100 | 0.011 | |
| 101 | 0.007 | |
| 102 | 0.012 | |
| 103 | 0.009 | |
| 104 | <0.004 | |
| 105 | 0.012 | |
| 106 | 0.125 | |
| 107 | 0.026 | |
| 108 | 0.025 | |
| 110 | 0.452 | |
| 111 | 0.144 | |
| 112 | 0.023 | |
| 113 | 0.061 | |
| 114 | 0.191 | |
| 115 | 0.023 | |
| 116 | 0.011 | |
| 117 | 0.007 | |
| 118 | 0.045 | |
| 119 | 0.012 | |
| 120 | 0.027 | |
| 121 | 0.012 | |
| 122 | 0.060 | |
| 123 | 0.033 | |

To support the unexpected activity of the compounds of the present application with respect to closest compounds described in the prior art application WO2012/139930, we reported in the Table B below the structure and biochemical data on Syk assay obtained for three reference compounds (namely comp. 7, 14 and 39).

TABLE B

| | Ref. Comp. | IC50 (μM) |
|---|---|---|
| Structure | number | Kglo |
| 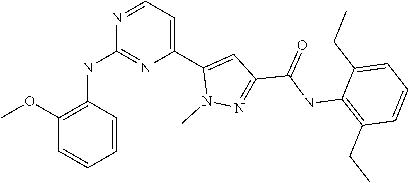 [SMILES: CCc1cccc(CC)c1NC(=O)c1cc(-c2ccnc(Nc3ccccc3OC)n2)n(C)n1] | 7 | >10 |
| 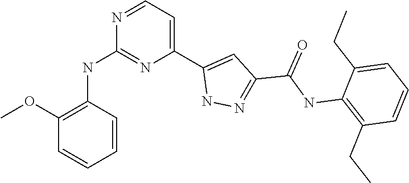 [SMILES: CCc1cccc(CC)c1NC(=O)c1cc(-c2ccnc(Nc3ccccc3OC)n2)[nH]n1] | 14 | >10 |
| 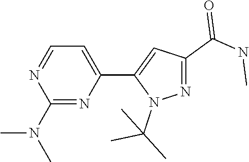 | 39 | >10 |

From the above data, it is clear to the person skilled in the art that while the compounds of formula (I) of the present invention are highly potent on Syk kinase, the compounds of the prior art can be considered inactive.

1. A compound of formula (I): 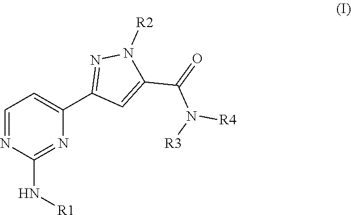
wherein:
R1 is hydrogen or an optionally substituted group selected from straight or branched (C1-C6) alkyl, (C3-C7) cycloalkyl, aryl, heterocyclyl and heteroaryl;
R2 is hydrogen or an optionally substituted group selected from straight or branched (C1-C6) alkyl, (C2-C6) alkenyl, (C2-C6) alkynyl, and (C3-C7) cycloalkyl;
R3 and R4 are independently hydrogen, straight or branched (C1-C6) alkyl optionally substituted with halogens, heteroaryl or heteroaryl (C1-C6) alkyl or a group of formula (II): 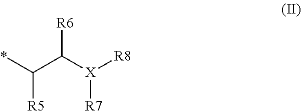
wherein:
R5 is hydrogen, an optionally substituted straight or branched (C1-C6) alkyl, (C3-C7) cycloalkyl, aryl or taken together with R6 may form an optionally substituted 4- to 7 membered cycloalkyl or, taken together with R7 or R8, may form an optionally substituted heterocyclyl group;
R6 is hydrogen or methyl or taken together with R3 or R4 may form an optionally substituted 4- to 7 membered heterocyclic group;
R7 and R8 are independently hydrogen, an optionally substituted straight or branched (C1-C6) alkyl or may form together with X an optionally substituted 4- to 7 membered heterocyclyl group optionally containing one additional heteroatom selected from N, O and S or may form together with R3 or R4 an optionally substituted 5- to 7 membered heterocyclyl group;
X is H, N or O
provided that, when X is O, R5 is not phenyl;
or a pharmaceutically acceptable salt thereof.

2. A compound of formula (I) according to claim 1 or a pharmaceutically acceptable salt thereof wherein:
R2 is an optionally substituted straight or branched (C1-C6) alkyl;
R3 and R4 are independently hydrogen or a group of general formula (II) 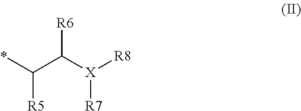
wherein:
R5 is hydrogen, an optionally substituted straight or branched (C1-C6)alkyl, (C3-C7)cycloalkyl, phenyl or, taken together with R6 may form an optionally substituted 4- to 7 membered cycloalkyl;
R6 is hydrogen;
R7 and R8 are independently hydrogen, an optionally substituted straight or branched (C1-C6)alkyl or may form together with X an optionally substituted 4- to 7 membered heterocyclyl group optionally containing one additional heteroatom selected from N, O and S;
X is N or O;
R1 is as defined in claim 1;
provided that, when X is O, R5 is not phenyl.

3. A compound of formula (I) according to claim 1 or a pharmaceutically acceptable salt thereof wherein:
R1 is hydrogen or an optionally substituted group selected from straight or branched (C1-C6) alkyl, (C3-C7) cycloalkyl, aryl, heterocyclyl and heteroaryl;
R2 is an optionally substituted straight or branched (C1-C6) alkyl;
R3 is hydrogen or straight or branched (C1-C6) alkyl optionally substituted with halogens, heteroaryl or heteroaryl (C1-C6) alkyl;
R4 is hydrogen or a group of general formula (II) 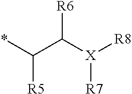
R5 is hydrogen, an optionally substituted straight or branched (C1-C6)alkyl, (C3-C7)cycloalkyl, phenyl or, taken together with R6 may form an optionally substituted 4- to 7 membered cycloalkyl;
R6 is hydrogen;
R7 and R8 are independently hydrogen, an optionally substituted straight or branched (C1-C6)alkyl or may form together with X an optionally substituted 4- to 7 membered heterocyclyl group optionally containing one additional heteroatom selected from N, O and S;
X is N or O;
provided that, when X is O, R5 is not phenyl.

4. A compound of formula (I) according to claim 1 or a pharmaceutically acceptable salt thereof, wherein:
R1 is an optionally substituted aryl, heterocyclyl or heteroaryl group;
R3 is hydrogen;
R4 is a group of general formula (II): 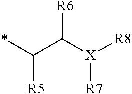
wherein:
R5 is hydrogen, an optionally substituted straight or branched (C1-C6)alkyl, (C3-C7)cycloalkyl, phenyl or, taken together with R6 may form an optionally substituted 4- to 7 membered cycloalkyl;
R6 is hydrogen;
R7 and R8 are independently hydrogen, an optionally substituted straight or branched (C1-C6)alkyl or may form together with X an optionally substituted 4- to 7 membered heterocyclyl;
X is N;
R2 is hydrogen or an optionally substituted group selected from straight or branched (C1-C6) alkyl, (C2-C6) alkenyl, (C2-C6) alkynyl, and (C3-C7) cycloalkyl.

5. A compound of formula (I) according to claim 4 or a pharmaceutically acceptable salt thereof, wherein:
R1 is an optionally substituted group selected from phenyl or indolyl.

6. A compound of formula (I) or a pharmaceutically acceptable salt thereof according to claim 1, selected from the group consisting of:
N-[2-(dimethylamino)ethyl]-3-{2-[(3,5-dimethylphenyl)amino]pyrimidin-4-yl}-1-methyl-1H-pyrazole-5-carboxamide (cpd 1);
3-{2-[(3,5-dimethylphenyl)amino]pyrimidin-4-yl}-N-[(2R)-1-hydroxypropan-2-yl]-1-methyl-1H-pyrazole-5-carboxamide (cpd 2);
3-{2-[(3,5-dimethylphenyl)amino]pyrimidin-4-yl}-N-[(2S)-1-hydroxypropan-2-yl]-1-methyl-1H-pyrazole-5-carboxamide (cpd 3);
3-{2-[(3,5-dimethylphenyl)amino]pyrimidin-4-yl}-1-methyl-N-(propan-2-yl)-1H-pyrazole-5-carboxamide (cpd 4);
N-[2-(dimethylamino)ethyl]-3-{2-[(3,5-dimethylphenyl)amino]pyrimidin-4-yl}-N,1-dimethyl-1H-pyrazole-5-carboxamide (cpd 5);
3-{2-[(3,5-dimethylphenyl)amino]pyrimidin-4-yl}-1-methyl-N-[2-(methylamino)ethyl]-1H-pyrazole-5-carboxamide hydrochloride (cpd 6);
N-(2-aminoethyl)-3-{2-[(3,5-dimethylphenyl)amino]pyrimidin-4-yl}-1-methyl-1H-pyrazole-5-carboxamide hydrochloride (cpd 7);
N-(azetidin-3-yl)-3-{2-[(3,5-dimethylphenyl)amino]pyrimidin-4-yl}-1-methyl-1H-pyrazole-5-carboxamide trifluoroacetate (cpd 8);
3-{2-[(3,5-dimethylphenyl)amino]pyrimidin-4-yl}-1-methyl-N-[2-(morpholin-4-yl)ethyl]-1H-pyrazole-5-carboxamide (cpd 9);
N-[2-(diethylamino)ethyl]-3-{2-[(3,5-dimethylphenyl)amino]pyrimidin-4-yl}-1-methyl-1H-pyrazole-5-carboxamide (cpd 10);
N-[(1R,2R)-2-aminocyclohexyl]-3-{2-[(3,5-dimethylphenyl)amino]pyrimidin-4-yl}-1-methyl-1H-pyrazole-5-carboxamide hydrochloride (cpd 11);
3-{2-[(3,5-dimethylphenyl)amino]pyrimidin-4-yl}-1-methyl-N-[2-(propan-2-ylamino)ethyl]-1H-pyrazole-5-carboxamide hydrochloride (cpd 12);
3-{2-[(3,5-dimethylphenyl)amino]pyrimidin-4-yl}-1-methyl-1H-pyrazole-5-carboxamide (cpd 13);
3-{2-[(3,5-dimethylphenyl)amino]pyrimidin-4-yl}-N,1-dimethyl-1H-pyrazole-5-carboxamide (cpd 14);
3-{2-[(3,5-dimethylphenyl)amino]pyrimidin-4-yl}-N,N,1-trimethyl-1H-pyrazole-5-carboxamide (cpd 15);
3-{2-[(3,5-dimethylphenyl)amino]pyrimidin-4-yl}-N-(2-methoxyethyl)-1-methyl-1H-pyrazole-5-carboxamide (cpd 16);
3-{2-[(3,5-dimethylphenyl)amino]pyrimidin-4-yl}-N-(2-fluoroethyl)-1-methyl-1H-pyrazole-5-carboxamide (cpd 17);
3-(2-{[3,5-bis(trifluoromethyl)phenyl]amino}pyrimidin-4-yl)-N-[2-(dimethylamino)ethyl]-1-methyl-1H-pyrazole-5-carboxamide hydrochloride (cpd 18);
3-(2-{[3,5-bis(trifluoromethyl)phenyl]amino}pyrimidin-4-yl)-N-[(2S)-1-hydroxypropan-2-yl]-1-methyl-1H-pyrazole-5-carboxamide (cpd 19);
3-{2-[(3,5-dimethylphenyl)amino]pyrimidin-4-yl}-1-methyl-N-(1H-pyrazol-3-yl)-1H-pyrazole-5-carboxamide (cpd 20);
(3-{2-[(3,5-dimethylphenyl)amino]pyrimidin-4-yl}-1-methyl-1H-pyrazol-5-yl)(4-methylpiperazin-1-yl)methanone (cpd 21);
N-[2-(acetylamino)ethyl]-3-{2-[(3,5-dimethylphenyl)amino]pyrimidin-4-yl}-1-methyl-1H-pyrazole-5-carboxamide (cpd 22);
N-(2-amino-2-oxoethyl)-3-{2-[(3,5-dimethylphenyl)amino]pyrimidin-4-yl}-1-methyl-1H-pyrazole-5-carboxamide (cpd 23);
3-{2-[(3,5-dimethylphenyl)amino]pyrimidin-4-yl}-1-methyl-N-(2,2,2-trifluoroethyl)-1H-pyrazole-5-carboxamide (cpd 24);
3-{2-[(3,5-dimethylphenyl)amino]pyrimidin-4-yl}-1-methyl-N-[(1-methyl-1H-imidazol-5-yl)methyl]-1H-pyrazole-5-carboxamide (cpd 25);
3-{2-[(3,5-dimethylphenyl)amino]pyrimidin-4-yl}-N-[(2S)-1-hydroxy-3-methylbutan-2-yl]-1-methyl-1H-pyrazole-5-carboxamide (cpd 26);
3-{2-[(3,5-dimethylphenyl)amino]pyrimidin-4-yl}-1-methyl-N-(pyridin-2-ylmethyl)-1H-pyrazole-5-carboxamide (cpd 27);
3-{2-[(3,5-dimethylphenyl)amino]pyrimidin-4-yl}-N-(1H-imidazol-2-ylmethyl)-1-methyl-1H-pyrazole-5-carboxamide (cpd 28);
N-[(2R)-1-(azetidin-1-yl)propan-2-yl]-3-{2-[(3,5-dimethoxyphenyl)amino]pyrimidin-4-yl}-1-methyl-1H-pyrazole-5-carboxamide (cpd 29);
N-[(2S)-1-(dimethylamino)propan-2-yl]-1-methyl-3-[2-(tetrahydro-2H-pyran-4-ylamino)pyrimidin-4-yl]-1H-pyrazole-5-carboxamide (cpd 30);
N-(1-azabicyclo[2.2.2]oct-3-yl)-3-{2-[(3,5-dimethylphenyl)amino]pyrimidin-4-yl}-1-methyl-1H-pyrazole-5-carboxamide (cpd 31);
5-[2-(3,5-Dimethyl-phenylamino)-pyrimidin-4-yl]-2-methyl-2H-pyrazole-3-carboxylic acid ((1R,2R)-2-hydroxy-cyclohexyl)-amide (cpd 32);
3-{2-[(3,5-dimethylphenyl)amino]pyrimidin-4-yl}-N-[(2S)-1-hydroxybutan-2-yl]-1-methyl-1H-pyrazole-5-carboxamide (cpd 33);
3-{2-[(3,5-dimethylphenyl)amino]pyrimidin-4-yl}-N-(2-hydroxyethyl)-1-methyl-1H-pyrazole-5-carboxamide (cpd 34);
N-[(2S)-1-(azetidin-1-yl)propan-2-yl]-3-{2-[(3,4-dimethoxyphenyl)amino]pyrimidin-4-yl}-1-methyl-1H-pyrazole-5-carboxamide (cpd 35);
(3-{2-[(3,5-dimethylphenyl)amino]pyrimidin-4-yl}-1-methyl-1H-pyrazol-5-yl)(piperazin-1-yl)methanone hydrochloride (cpd 36);
N-[(1S,2R)-2-aminocyclohexyl]-3-(2-{[3-methoxy-5-(trifluoromethyl)phenyl]amino}pyrimidin-4-yl)-1-methyl-1H-pyrazole-5-carboxamide hydrochloride (cpd 37);
N-[(1S,2R)-2-aminocyclohexyl]-3-{2-[(3-chloro-1-methyl-1H-indol-5-yl)amino]pyrimidin-4-yl}-1-methyl-1H-pyrazole-5-carboxamide hydrochloride (cpd 38);
N-[(2S)-1-(azetidin-1-yl)propan-2-yl]-3-{2-[(3-methoxyphenyl)amino]pyrimidin-4-yl}-1-methyl-1H-pyrazole-5-carboxamide (cpd 39);
N-[(2S)-1-(azetidin-1-yl)propan-2-yl]-3-{2-[(3,5-difluorophenyl)amino]pyrimidin-4-yl}-1-methyl-1H-pyrazole-5-carboxamide (cpd 40);
N-[(2S)-1-hydroxy-3-methylbutan-2-yl]-1-methyl-3-{2-[(3-methylphenyl)amino]pyrimidin-4-yl}-1H-pyrazole-5-carboxamide (cpd 41);
3-{2-[(3,5-dimethylphenyl)amino]pyrimidin-4-yl}-1-methyl-N-[(2S)-1,1,1-trifluoropropan-2-yl]-1H-pyrazole-5-carboxamide (cpd 42);
N-[3-(dimethylamino)propyl]-3-{2-[(3,5-dimethylphenyl)amino]pyrimidin-4-yl}-1-methyl-1H-pyrazole-5-carboxamide (cpd 43);
(3-{2-[(3,5-dimethylphenyl)amino]pyrimidin-4-yl}-1-methyl-1H-pyrazol-5-yl)[(2S)-2-(propan-2-yl)aziridin-1-yl] methanone (cpd 44);
3-{2-[(3,5-dimethylphenyl)amino]pyrimidin-4-yl}-N-(2,2-dimethylpropyl)-1-methyl-1H-pyrazole-5-carboxamide (cpd 45);
3-{2-[(3,5-dimethylphenyl)amino]pyrimidin-4-yl}-1-methyl-N-(2-methylpropyl)-1H-pyrazole-5-carboxamide (cpd 46);
N-(cyclopropylmethyl)-3-{2-[(3,5-dimethylphenyl)amino]pyrimidin-4-yl}-1-methyl-1H-pyrazole-5-carboxamide (cpd 47);
N-[(2S)-1-(azetidin-1-yl)propan-2-yl]-3-{2-[(3-fluoro-5-methoxyphenyl)amino]pyrimidin-4-yl}-1-methyl-1H-pyrazole-5-carboxamide (cpd 48);
3-{2-[(4,6-dimethylpyridin-2-yl)amino]pyrimidin-4-yl}-N-[(2S)-1-hydroxy-3-methylbutan-2-yl]-1-methyl-1H-pyrazole-5-carboxamide (cpd 49);
3-{2-[(3,5-difluorophenyl)amino]pyrimidin-4-yl}-1-methyl-N-[(2S)-1,1,1-trifluoropropan-2-yl]-1H-pyrazole-5-carboxamide (cpd 50);
1-methyl-3-{2-[(3-methylphenyl)amino]pyrimidin-4-yl}-N-[(2S)-1,1,1-trifluoropropan-2-yl]-1H-pyrazole-5-carboxamide (cpd 51);
3-(2-{[3-chloro-4-(4-methylpiperazin-1-yl)phenyl]amino}pyrimidin-4-yl)-N-[(2S)-1-hydroxy-3-phenylpropan-2-yl]-1-methyl-1H-pyrazole-5-carboxamide (cpd 52);
N-[(1S,2S)-2-aminocyclohexyl]-1-methyl-3-{2-[(3-methylphenyl)amino]pyrimidin-4-yl}-1H-pyrazole-5-carboxamide (cpd 53);
3-(2-{[3-chloro-4-(4-methylpiperazin-1-yl)phenyl]amino}pyrimidin-4-yl)-1-methyl-N-[(2S)-1,1,1-trifluoropropan-2-yl]-1H-pyrazole-5-carboxamide (cpd 54);
3-{2-[(3,5-dimethylphenyl)amino]pyrimidin-4-yl}-1-methyl-N-[(2S)-1,1,1-trifluorobutan-2-yl]-1H-pyrazole-5-carboxamide (cpd 55);
3-{2-[(3,5-dimethylphenyl)amino]pyrimidin-4-yl}-1-methyl-N-[(2R)-1,1,1-trifluorobutan-2-yl]-1H-pyrazole-5-carboxamide (cpd 56);
N-[(2S)-1-(3,3-difluoroazetidin-1-yl)propan-2-yl]-3-{2-[(3,5-dimethylphenyl)amino]pyrimidin-4-yl}-1-methyl-1H-pyrazole-5-carboxamide (cpd 57);
1-methyl-N-[(2S)-1-(pyrrolidin-1-yl)propan-2-yl]-3-{2-[(3,4,5-trimethoxyphenyl)amino]pyrimidin-4-yl}-1H-pyrazole-5-carboxamide (cpd 58);
N-[(2S)-1-(azetidin-1-yl)propan-2-yl]-3-{2-[(3-chloro-1-methyl-1H-indol-5-yl)amino]pyrimidin-4-yl}-1-methyl-1H-pyrazole-5-carboxamide (cpd 59);
N-[(2S)-1-(azetidin-1-yl)propan-2-yl]-3-{2-[(3,5-dichlorophenyl)amino]pyrimidin-4-yl}-1-methyl-1H-pyrazole-5-carboxamide (cpd 60);
N-[(2S)-1-(azetidin-1-yl)-3-methylbutan-2-yl]-3-{2-[(3,5-dichlorophenyl)amino]pyrimidin-4-yl}-1-methyl-1H-pyrazole-5-carboxamide (cpd 61);
N-[(2S)-1-(azetidin-1-yl)propan-2-yl]-1-methyl-3-(2-{[3-(trifluoromethyl)phenyl]amino}pyrimidin-4-yl)-1H-pyrazole-5-carboxamide (cpd 62);
N-[(2S)-1-(azetidin-1-yl)propan-2-yl]-3-(2-{[3,5-bis(trifluoromethyl)phenyl]amino}pyrimidin-4-yl)-1-methyl-1H-pyrazole-5-carboxamide (cpd 63);
N-[(2S)-1-(azetidin-1-yl)propan-2-yl]-3-{2-[(3,5-dimethoxyphenyl)amino]pyrimidin-4-yl}-1-methyl-1H-pyrazole-5-carboxamide (cpd 64);
N-[(2S)-1-(dimethylamino)propan-2-yl]-3-{2-[(3-fluoro-5-methoxyphenyl)amino]pyrimidin-4-yl}-1-methyl-1H-pyrazole-5-carboxamide (cpd 65);
N-[(2S)-1-(azetidin-1-yl)propan-2-yl]-3-[2-(1,3-benzodioxol-5-ylamino)pyrimidin-4-yl]-1-methyl-1H-pyrazole-5-carboxamide (cpd 66);
3-{2-[(3,5-dimethoxyphenyl)amino]pyrimidin-4-yl}-N-[(2S)-1-(dimethylamino)propan-2-yl]-1-methyl-1H-pyrazole-5-carboxamide (cpd 67);
N-[(2S)-1-(azetidin-1-yl)propan-2-yl]-1-methyl-3-{2-[(3,4,5-trimethoxyphenyl)amino]pyrimidin-4-yl}-1H-pyrazole-5-carboxamide (cpd 68);
3-{2-[(3,5-dimethoxyphenyl)amino]pyrimidin-4-yl}-1-methyl-N-[(2S)-1-(pyrrolidin-1-yl)propan-2-yl]-1H-pyrazole-5-carboxamide (cpd 69);
3-{2-[(3-methoxyphenyl)amino]pyrimidin-4-yl}-1-methyl-N-[(2S)-1-(pyrrolidin-1-yl)propan-2-yl]-1H-pyrazole-5-carboxamide (cpd 70);
3-{2-[(3,5-dimethoxyphenyl)amino]pyrimidin-4-yl}-N-[(2S)-1-(dimethylamino)-1-oxopropan-2-yl]-1-methyl-1H-pyrazole-5-carboxamide (cpd 71);
3-{2-[(3,5-dimethoxyphenyl)amino]pyrimidin-4-yl}-N-[2-(dimethylamino)ethyl]-1-methyl-1H-pyrazole-5-carboxamide (cpd 72);
N-[(2S)-1-(dimethylamino)propan-2-yl]-3-{2-[(1,5-dimethyl-1H-pyrazol-3-yl)amino]pyrimidin-4-yl}-1-methyl-1H-pyrazole-5-carboxamide (cpd 73);
N-[(2S)-1-(azetidin-1-yl)propan-2-yl]-3-{2-[(1,5-dimethyl-1H-pyrazol-3-yl)amino]pyrimidin-4-yl}-1-methyl-1H-pyrazole-5-carboxamide (cpd 74);
N-[(2S)-1-(dimethylamino)propan-2-yl]-1-methyl-3-{2-[(1-methyl-1H-pyrazol-4-yl)amino]pyrimidin-4-yl}-1H-pyrazole-5-carboxamide (cpd 75);
N-[(2S)-1-(azetidin-1-yl)propan-2-yl]-1-methyl-3-[2-(tetrahydro-2H-pyran-4-ylamino)pyrimidin-4-yl]-1H-pyrazole-5-carboxamide (cpd 76);
N,N,1-trimethyl-3-[2-(tetrahydro-2H-pyran-4-ylamino)pyrimidin-4-yl]-1H-pyrazole-5-carboxamide (cpd 77);
3-{2-[(3-cyano-5-methoxyphenyl)amino]pyrimidin-4-yl}-N-[(2S)-1-(dimethylamino)propan-2-yl]-1-methyl-1H-pyrazole-5-carboxamide (cpd 78);
N-[(2S)-1-(azetidin-1-yl)propan-2-yl]-3-{2-[(3-cyano-5-methoxyphenyl)amino]pyrimidin-4-yl}-1-methyl-1H-pyrazole-5-carboxamide (cpd 79);
N-[(2S)-1-(azetidin-1-yl)propan-2-yl]-3-{2-[(5-methoxypyridin-3-yl)amino]pyrimidin-4-yl}-1-methyl-1H-pyrazole-5-carboxamide (cpd 80);
3-{2-[(3,5-dimethylphenyl)amino]pyrimidin-4-yl}-1-methyl-N-[(3S)-piperidin-3-yl]-1H-pyrazole-5-carboxamide hydrochloride (cpd 81);
N-(2-aminocyclohexyl)-3-{2-[(3,5-dimethylphenyl)amino]pyrimidin-4-yl}-1-methyl-1H-pyrazole-5-carboxamide hydrochloride (cpd 82);
3-{2-[(3,5-dimethylphenyl)amino]pyrimidin-4-yl}-1-methyl-N-[2-(pyrrolidin-1-yl)ethyl]-1H-pyrazole-5-carboxamide (cpd 83);
3-{2-[(3,5-dimethylphenyl)amino]pyrimidin-4-yl}-1-methyl-N-[2-(piperidin-1-yl)ethyl]-1H-pyrazole-5-carboxamide (cpd 84);
(3-{2-[(3,5-dimethylphenyl)amino]pyrimidin-4-yl}-1-methyl-1H-pyrazol-5-yl)[(3R)-3-hydroxypyrrolidin-1-yl]methanone (cpd 85);
[(3S)-3-(dimethylamino)pyrrolidin-1-yl](3-{2-[(3,5-dimethylphenyl)amino]pyrimidin-4-yl}-1-methyl-1H-pyrazol-5-yl)methanone (cpd 86);
[(3R)-3-(dimethylamino)pyrrolidin-1-yl](3-{2-[(3,5-dimethylphenyl)amino]pyrimidin-4-yl}-1-methyl-1H-pyrazol-5-yl)methanone (cpd 87);
(3-aminopyrrolidin-1-yl)(3-{2-[(3,5-dimethylphenyl)amino]pyrimidin-4-yl}-1-methyl-1H-pyrazol-5-yl)methanone (cpd 88);
(3-{2-[(3,5-dimethylphenyl)amino]pyrimidin-4-yl}-1-methyl-1H-pyrazol-5-yl)[(2S)-2-(hydroxymethyl)pyrrolidin-1-yl]methanone (cpd 89);
(3-{2-[(3,5-dimethylphenyl)amino]pyrimidin-4-yl}-1-methyl-1H-pyrazol-5-yl)[(3S)-3-hydroxypyrrolidin-1-yl]methanone (cpd 90);
3-{2-[(3,5-dimethylphenyl)amino]pyrimidin-4-yl}-N-[(1R,2S)-2-hydroxycyclohexyl]-1-methyl-1H-pyrazole-5-carboxamide (cpd 91);
N-[(1S,2R)-2-aminocyclohexyl]-3-{2-[(3,5-dimethylphenyl)amino]pyrimidin-4-yl}-1-methyl-1H-pyrazole-5-carboxamide hydrochloride (cpd 92);
N-[(2S)-1-hydroxypropan-2-yl]-1-methyl-3-{2-[(1,2,3-trimethyl-1H-indol-5-yl)amino]pyrimidin-4-yl}-1H-pyrazole-5-carboxamide (cpd 93);
3-{2-[(3,5-dimethylphenyl)amino]pyrimidin-4-yl}-1-methyl-N-[(2S)-1-(pyrrolidin-1-yl)propan-2-yl]-1H-pyrazole-5-carboxamide (cpd 94);
N-[(2S)-1-(azetidin-1-yl)propan-2-yl]-3-{2-[(3,5-dimethylphenyl)amino]pyrimidin-4-yl}-1-methyl-1H-pyrazole-5-carboxamide (cpd 95);
(1R,4S)-2,5-diazabicyclo[2.2.1]hept-2-yl(3-{2-[(3,5-dimethylphenyl)amino]pyrimidin-4-yl}-1-methyl-1H-pyrazol-5-yl)methanone hydrochloride (cpd 96);
3-{2-[(3,5-dimethylphenyl)amino]pyrimidin-4-yl}-1-methyl-N-[(3R)-piperidin-3-yl]-1H-pyrazole-5-carboxamide hydrochloride (cpd 97);
N-[(1R,2S)-2-aminocyclohexyl]-3-{2-[(3,5-dimethylphenyl)amino]pyrimidin-4-yl}-1-methyl-1H-pyrazole-5-carboxamide hydrochloride (cpd 98);
N-[(1S,2R)-2-aminocyclohexyl]-3-{2-[(3,5-dichlorophenyl)amino]pyrimidin-4-yl}-1-methyl-1H-pyrazole-5-carboxamide hydrochloride (cpd 99);
N-[(1S,2R)-2-aminocyclohexyl]-3-{2-[(3,5-dimethoxyphenyl)amino]pyrimidin-4-yl}-1-methyl-1H-pyrazole-5-carboxamide hydrochloride (cpd 100);
N-[(1S,2S)-2-aminocyclohexyl]-3-{2-[(3,5-dimethylphenyl)amino]pyrimidin-4-yl}-1-methyl-1H-pyrazole-5-carboxamide (cpd 101);
N-[(2S)-1-(azetidin-1-yl)-3-methylbutan-2-yl]-3-{2-[(3,5-dimethylphenyl)amino]pyrimidin-4-yl}-1-methyl-1H-pyrazole-5-carboxamide (cpd 102);
3-{2-[(3,5-dimethylphenyl)amino]pyrimidin-4-yl}-1-methyl-N-[(2S)-3-methyl-1-(pyrrolidin-1-yl)butan-2-yl]-1H-pyrazole-5-carboxamide (cpd 103);
N-[(1S,2R)-2-aminocyclohexyl]-3-{2-[(3,5-dimethylphenyl)amino]pyrimidin-4-yl}-1H-pyrazole-5-carboxamide hydrochloride (cpd 104);
N-[(2S)-1-(azetidin-1-yl)-3-methylbutan-2-yl]-3-{2-[(3,5-dimethylphenyl)amino]pyrimidin-4-yl}-1H-pyrazole-5-carboxamide (cpd 105);
N-[(1S)-1-cyclohexyl-2-hydroxyethyl]-3-{2-[(3,5-dimethylphenyl)amino]pyrimidin-4-yl}-1-methyl-1H-pyrazole-5-carboxamide (cpd 106);
3-{2-[(3,5-dimethylphenyl)amino]pyrimidin-4-yl}-1-methyl-N-[(1S)-1-phenyl-2-(pyrrolidin-1-yl)ethyl]-1H-pyrazole-5-carboxamide (cpd 107);
N-[(2S)-1-(azetidin-1-yl)propan-2-yl]-1-methyl-3-{2-[(3-methylphenyl)amino]pyrimidin-4-yl}-1H-pyrazole-5-carboxamide (cpd 108);
3-{2-[(3,5-dimethylphenyl)amino]pyrimidin-4-yl}-N-[(2S)-1-hydroxy-3-phenylpropan-2-yl]-1-methyl-1H-pyrazole-5-carboxamide (cpd 109);
3-(2-{[3-chloro-4-(4-methylpiperazin-1-yl)phenyl]amino}pyrimidin-4-yl)-N-[(2S)-1-hydroxypropan-2-yl]-1-methyl-1H-pyrazole-5-carboxamide (cpd 110);
3-(2-{[3-chloro-4-(4-methylpiperazin-1-yl)phenyl]amino}pyrimidin-4-yl)-N-[(2S)-1-hydroxy-3-methylbutan-2-yl]-1-methyl-1H-pyrazole-5-carboxamide (cpd 111);
1-methyl-3-{2-[(3-methylphenyl)amino]pyrimidin-4-yl}-N-[(2S)-3-methyl-1-(pyrrolidin-1-yl)butan-2-yl]-1H-pyrazole-5-carboxamide (cpd 112);
N-[(1S,2S)-2-aminocyclohexyl]-3-{2-[(3,5-difluorophenyl)amino]pyrimidin-4-yl}-1-methyl-1H-pyrazole-5-carboxamide (cpd 113);
3-(2-{[3-chloro-4-(4-methylpiperazin-1-yl)phenyl]amino}pyrimidin-4-yl)-N-[(1S,2R)-2-hydroxycyclohexyl]-1-methyl-1H-pyrazole-5-carboxamide (cpd 114);
3-{2-[(3,5-dimethylphenyl)amino]pyrimidin-4-yl}-N-[(1S,2R)-2-hydroxycyclohexyl]-1-methyl-1H-pyrazole-5-carboxamide (cpd 115);
N-[(1S,2S)-2-aminocyclohexyl]-3-{2-[(3,5-dichlorophenyl)amino]pyrimidin-4-yl}-1-methyl-1H-pyrazole-5-carboxamide (cpd 116);
3-{2-[(3,5-dichlorophenyl)amino]pyrimidin-4-yl}-1-methyl-N-[(2S)-3-methyl-1-(pyrrolidin-1-yl)butan-2-yl]-1H-pyrazole-5-carboxamide (cpd 117);
3-{2-[(3,5-dimethylphenyl)amino]pyrimidin-4-yl}-N-[(1S,2S)-2-hydroxycyclohexyl]-1-methyl-1H-pyrazole-5-carboxamide (cpd 118);
N-[(2S)-1-(azetidin-1-yl)propan-2-yl]-3-{2-[(3-cyanophenyl)amino]pyrimidin-4-yl}-1-methyl-1H-pyrazole-5-carboxamide (cpd 119);
3-{2-[(3,5-dimethylphenyl)amino]pyrimidin-4-yl}-1-ethyl-N-[(1S,2R)-2-hydroxycyclohexyl]-1H-pyrazole-5-carboxamide (cpd 120);
N-[(1S,2R)-2-aminocyclohexyl]-3-{2-[(3,5-dimethylphenyl)amino]pyrimidin-4-yl}-1-ethyl-1H-pyrazole-5-carboxamide hydrochloride (cpd 121);
3-{2-[(3,5-dimethylphenyl)amino]pyrimidin-4-yl}-1-ethyl-N-[(3R)-piperidin-3-yl]-1H-pyrazole-5-carboxamide hydrochloride (cpd 122) and
N-[(2S)-1-(azetidin-1-yl)propan-2-yl]-3-{2-[(3,5-dimethoxyphenyl)amino]pyrimidin-4-yl}-N,1-dimethyl-1H-pyrazole-5-carboxamide (cpd 123).

7. A process for the preparation of a compound of formula (I) or a pharmaceutical acceptable salt thereof, as defined in claim 1, said process comprises:
st. 1) mixing the compound of formula (III): 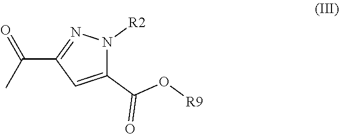
wherein, R2 as defined in claim 1 and R9 is a group selected from straight or branched (C1-C6) alky, with a dimethylformamide-dialkylacetale;
st. 2) reacting the resultant compound of formula (IV): 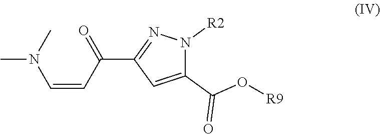
wherein R2 and R9 are as defined above, with a compound of formula (V): 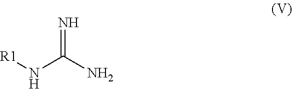
wherein R1 is as defined in claim 1, so as to obtain a compound of formula (V) 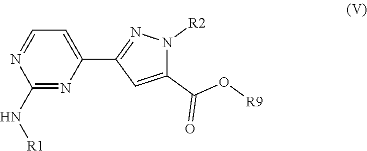
wherein R1, R2 and R9 are as defined as above;
alternatively compound of formula (V), as reported under step 2, can prepared according to the steps below:
st. 3) reacting the formula (IV): 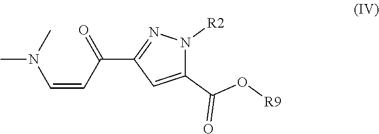
wherein R2 and R9 are as defined above in step 1), with guanidine carbonate;
st. 4) reacting the resultant compound of formula (VII) 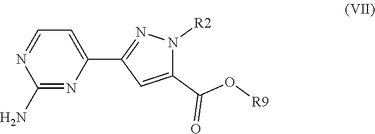
wherein R2 and R9 are as defined above, with a compound of formula (VIII): 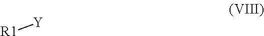
wherein R1 is as defined in claim 1 and Y is iodine or bromine in presence of Palladium, so as to obtain a compound of formula (VI) 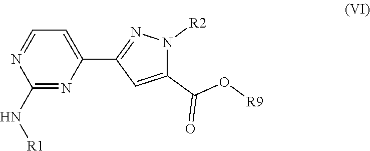
wherein R1, R2 and R9 are as defined above;
or
st. 5) reacting the resultant compound of formula (VII) 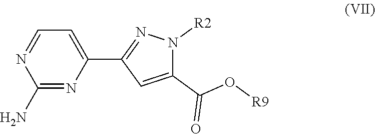
obtained as reported under step 3), by reaction with iso-amylnitrite and diiodomethane or cesium iodide, in the presence of iodine and CuI, thus to obtain a compound of formula (IX) wherein R2 and R9 are as defined above;
st. 6) then, reacting the resultant compound of formula (IX) 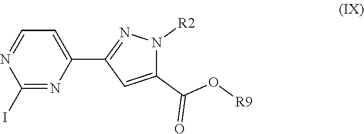
wherein R2 and R9 are as defined above, with a compound of formula (X): 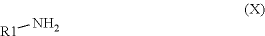
wherein R1 is as defined in claim 1, in presence of Palladium, so as to obtain a compound of formula (VI) 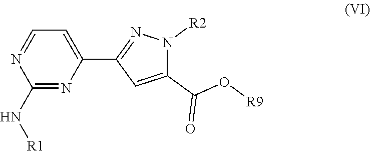
wherein R1, R2 and R9 are as defined as above;
the compound of formula (I) can be prepared accordingly to a process comprises the following steps:
st. 7) reacting the compound of formula (VI) obtained as described in steps 2, 4 or 6 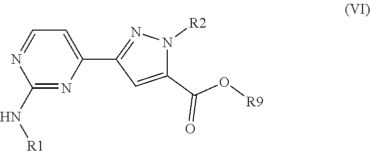
wherein, R1 and R2 are as defined in claim 1 and R9 is a group selected from straight or branched (C1-C6) alky, in acid or basic hydrolysis conditions as to obtain a compound of formula (XI) or the corresponding salt; 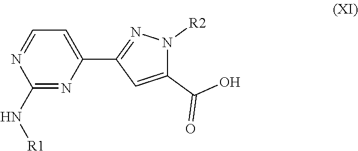
wherein R1 and R2 are as defined as above;
st. 8) reacting the compound of formula (XI) or the corresponding salt as described in steps 7 with compound of formula (XII) 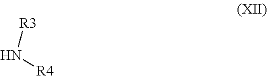
wherein R3 and R4 are as defined in claim 1, thus to obtain a compound of formula (I) 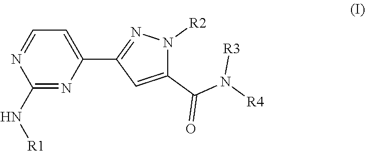
wherein R1, R2, R3 and R4 are as defined in claim 1.

8.-12. (canceled)

13. A method of treating a disease caused by and/or associated with dysregulated Syk kinase activity which comprises administering to a mammal, preferably a human, in need thereof, an effective amount of a compound of formula (I) as defined in claim 1.

14. The method, according to claim 13, wherein the disease is selected from the group consisting of cancer, cell proliferative disorders and immune-related disorders.

15. The method, according to claim 14, wherein the disease is cancer.

16. The method according to claim 15, wherein said cancer is selected from the group consisting of: carcinomas, such as carcinomas, such as carcinomas, such as bladder, breast, kidney, liver, colon, lung, including small cell lung cancer, esophagus, gall-bladder, ovary, pancreas, stomach, cervix, prostate, and skin, including squamous cell carcinoma; hematopoietic tumors of lymphoid lineage including leukemia, acute lymphocitic leukemia, acute lymphoblastic leukemia, chronic lymphocytic leukemia, B-cell lymphoma, angioimmunoblastic T-cell lymphoma, Hodgkin's lymphoma, non-Hodgkin's lymphoma, hairy cell lymphoma and Burkitt's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including glioma, glioblastoma, glioblastoma multiforme, astrocytoma, oligodendroglioma, paraglioma, neuroblastoma, and schwannomas; and other tumors, including melanoma, seminoma, teratocarcinoma, osteosarcoma, xeroderma pigmentosum, keratoxanthoma, thyroid cancers, such as papillary thyroid carcinoma and medullary thyroid carcinoma, Kaposi's sarcoma, chondrosarcoma, and cholangiocarcinoma.

17. A pharmaceutical composition comprising a compound of formula (I) or a pharmaceutically acceptable salt thereof, as defined in claim 1, in association with a pharmaceutically acceptable excipient, carrier or diluent.

18. A pharmaceutical composition according to claim 17 further comprising one or more chemotherapeutic agents.

19. A product or kit comprising a compound of formula (I) or a pharmaceutically acceptable salt thereof, as defined in claim 1, and one or more chemotherapeutic agents, as a combined preparation for simultaneous, separate or sequential use in anticancer therapy.

20. (canceled)

21. The method according to claim 13 in combination with radiation therapy or with a chemotherapy regimen.
